(19) United States
(12) Patent Application Publication
(10) Pub. No.: US 2021/0395819 A1
(43) Pub. Date: Dec. 23, 2021

(54) METHODS AND DEVICES FOR RAPID DETECTION OF TARGET GENETIC MATERIAL

(71) Applicant: The Chinese University of Hong Kong, Hong Kong (CN)

(72) Inventors: Yinyin Liu, Hong Kong (CN); Ho Sing Lo, Hong Kong (CN); Hoi Lam Elsa Yeung, Hong Kong (CN); Nga Yan Chan, Hong Kong (CN); Yunfan Geng, Hong Kong (CN); Hiu Yan Wong, Hong Kong (CN); Yuwei Guo, Hong Kong (CN); Man Long Kwok, Hong Kong (CN); Siu Kai Kong, Hong Kong (CN); King Ming Chan, Hong Kong (CN); Ting Fung Chan, Hong Kong (CN)

(21) Appl. No.: 17/064,616

(22) Filed: Oct. 7, 2020

Related U.S. Application Data
(60) Provisional application No. 62/911,901, filed on Oct. 7, 2019.

(51) Int. Cl. C12Q 1/6876; G06N 20/00; C12Q 1/6825

(52) U.S. Cl. CPC C12Q 1/6876; G06N 20/00; G01N 2333/165; G01N 2800/26; C12Q 1/6825

(57) ABSTRACT

The present invention provides RNA aptamer probes for detection of target genetic material and methods for using the probes. In some embodiments, the invention provides devices for the detection of the target genetic material using the probes of the preset invention. In some embodiments, the invention provides methods for designing RNA aptamer probes for detection of target genetic material. In some embodiments, the target genetic material is genetic material from a pathogen. In some embodiments the pathogen is influenza virus. In some embodiments, the devices of the present invention may be used outside of laboratory setting and do not require any specialized skills. In some embodiments, the devices of the present invention are used in conjunction with a mobile phone camera.

### FIELD OF THE INVENTION

This invention relates generally to RNA-based detection of viruses. In particular, this invention is related to RNA-based compositions, methods and devices for rapid detection of influenza viruses.

### BACKGROUND OF THE INVENTION

Over the past few centuries, respiratory diseases caused by RNA virus have caused global epidemics. The notorious COVID2019, SARS, MERS, Spanish Flu are all in this kind, and they take away the lives of millions of people every time they break out.

Communicable diseases such as influenza A have been a life-threatening issue that could take away lives of millions of people, especially for old people, young children, and patients with chronic diseases. In 1957, Asian flu took away more than 2 million lives. In 1968, Hong Kong flu caused 1 million deaths worldwide. Over the past years, in Hong Kong alone, thousands of people died because of influenza, and the number is increasing every year.

However, the symptoms of cold and influenza are hard to distinguish. In a survey which collected about 300 responses from China, Hong Kong or Singapore, more than 70% of the respondents could not differentiate between cold and flu, and around 50% stated that they do not seek medical help when they get flu-like symptoms. Inaccurate diagnosis of influenza leads to inappropriate or delayed treatment of flu-like symptoms and puts lives of patients and communities in danger.

Moreover, currently, many healthcare systems for epidemic diseases are highly centralized, which means that people can only receive testing and treatment at certain hospitals and clinics. This traditional system is highly vulnerable and may even collapse when dealing with respiratory infectious diseases, as they often break out in large volumes and high densities.

Therefore, there is a need to develop a cheap, rapid, accurate and convenient tool for detection of influenza which permits on-site detection of influenza by the general public. The tool will also allow patients to monitor their infectious status on a regular basis throughout treatment.

At the time of this invention, there are two most widely used ways to detect influenza for clinical purposes: one is quantitative polymerase chain reaction (qPCR) (Patel P. 2011), another one is rapid tests using influenza-specific antibody (e.g. ID NOW™ Influenza A & B 2 assay from Abbott).

qPCR can be used to detect RNA of viruses for the purpose of detection or identification. It has high accuracy but requires expertise and must be conducted in a laboratory setting. Moreover, it takes a long time (around 6 hours) to complete the testing and therefore not suitable for on-site testing.

Rapid tests using influenza-specific antibodies such as enzyme-linked immunosorbent assay (ELISA) are also used in clinics. However, antibody-based tests are often more expensive and less specific than the nucleic acid-based method. For instance, the current rapid tests use color change on the test paper to indicate the testing results. Due to the difficulty of recognizing different colors by human eyes, the false positive rate is as high as 30% to 50% (Nie, 2014).

A relatively new approach for high-throughput screening and rapid detection of pathogens including influenza viruses is “toehold switch” which is an RNA probe complementary to the target RNA with high specificity that releases ribosome binding site (Green A A, 2014). This tool overcomes the limitations of the qPCR and rapid antibody methods in terms of time and location, and provides a preliminary tool for pandemic control (Pardee K, 2016). This technique, however, is yet to be commercialized. Past toehold switch designs utilized fluorescent proteins and hydrolases (e.g. lacZ) as reporters to balance between detection accuracy and sensitivity (Green A A, 2014; Pardee K, 2016; CUHK iGEM Team, 2017), and have several limitations such as specific spectral requirements for fluorescence detection, high costs of enzyme substrates and long waiting time which could be as long as 4 hours (Pardee K, 2016).

RNA aptamer is a single-strand RNA molecule that can bind to a specific target. With higher thermal stability, smaller size, shorter developing time, aptamers are believed to be an alternative to antibody, especially in the field of diagnostics. Although rapid tests using RNA probes such as RNA aptamer probes (RAPID) offer advantages over rapid tests using antibodies, such as lower costs and higher specificity, the challenge of using RNA aptamers is that aptamers are harder to design and even if specific aptamers are successfully designed, the test is not very affordable for the general public because the production cost of aptamers remain relatively high. Thus it is desirable to develop a tool for optimization of the aptamer sequence design and methods for mass production and screening of aptamers such as using bacterial system which may reduce the cost to 1-2 US dollar per test.

In view of the foregoing, the present invention provides RNA-based compositions, methods and devices that are capable of rapid and accurate detection of influenza viruses outside of laboratory setting, thereby providing a more convenient and affordable testing. With this tool, the pressure of healthcare system during epidemic seasons is not only expected to be reduced, but also able to facilitate large-scale screening, as aptamers are much easier and thus cheaper to produce compared to antibody and rt-PCR.

### SUMMARY OF THE INVENTION

The present invention provides compositions, methods, devices and systems for detecting target gene sequences using fluorescent RNA aptamer probes. The present invention may be used for detecting influenza viruses but can be adapted for detection of other types of pathogenic organisms or other genetic material.

In one embodiment, the present invention provides RNA aptamer probes which specifically bind to gene sequences of a certain type or subtype of influenza virus and, in the presence of certain fluorogens, produce detectable fluorescent signals upon binding to the target gene sequences. In some embodiments, the RNA aptamer probes emit no or negligible fluorescence in the absence of their respective target RNA sequences. Upon binding to their respective target RNA sequences, the RNA aptamer probes change their confirmation which enables them to interact with a fluorogen in a way that induces fluorescence or leads to an increase in intensity of the fluorescence produced by the complex. It is to be understood that when RNA aptamer probes are described herein as fluorescing, emitting fluorescent light or fluorescence, the RNA aptamer probe refers to the RNA aptamer in complex with the fluorogen.

In one embodiment, the present invention provides a method or system for designing RNA aptamer probes for detecting genetic materials of a particular type or subtype of influenza virus or another organism.

In some embodiments, the present invention provides a software that may be used to design RNA aptamer probes. In some embodiments, the system is equipped with a neural network that trains the processing ability of the system in differentiating between positive and negative signals.

In one embodiment, the present invention provides a device for detecting and processing light or fluorescent signals produced by the present RNA aptamer probes or other light-emitting moieties which indicate the presence of target organisms or their genetic material. In some embodiments the devices are battery operated and may be used in conjunction with mobile phone cameras for detecting the fluorescent signal.

In one embodiment, the present invention provides an integrated system for a subject self-test for of influenza virus outside of laboratory setting. In some embodiments, the present integrated system comprises one or more of: a module for collecting a sample of nasal fluid from a subject, a module for treating the collected sample with a detecting reagent comprising one or more fluorogen-bearing influenza-specific probes, a light-shielded module for taking one or more images recording light emitted from the treated sample, and a module for processing the images and outputting results indicating the presence or absence of particular type or subtype of influenza virus. In some embodiments, the present integrated system is linked with a mobile phone of the user and configured to enable the user to take images of their samples using their mobile phones and upload the images to the present integrated system for image-processing and analysis, and to receive results from the integrated system via the mobile phone.

In one embodiment, the present invention provides a system for detecting and processing light or fluorescent signals given out by the present RNA aptamer probes or other light-emitting moieties which indicate the presence of target organisms or their genetic material. In some embodiments, the system is equipped with a neural network that trains the processing ability of the system in differentiating between positive and negative signals.

Various embodiments of the present invention may be used to collect data for monitoring and control of influenza as well as data that may be used for improvement of the design of the probes representing some embodiments of the invention. In some embodiments, machine learning may be used for probe design and for data analysis.

In some embodiments, the methods, systems and devices of the present invention may be used to detect genetic material of various pathogenic organisms or other genetic material of interest.

### BRIEF DESCRIPTION OF THE DRAWINGS

FIG. 1 is a flowchart depicting how various embodiments of the present invention may be used to collect data for monitoring and control of influenza as well as data that may be used for improvement of the design of the probes representing some embodiments of the invention.

FIG. 2 is a flowchart schematically depicting the data processing steps of some embodiments of the present invention starting with taking a photograph of the sample.

FIG. 3 illustrates the working mechanism of one embodiment of RNA aptamer probes provided by this invention.

FIG. 4 shows how some embodiments of the present RNA aptamer probe may be designed using BLOCK-iT Designer.

FIG. 5 is a schematic diagram of in vitro transcription of the present RNA aptamer probes and RNA targets presenting one embodiment of the present invention.

FIGS. 6A-6C shows the results of aptamer refolding assay obtained from some embodiments of the present aptamer probes (N=3, *:p<0.05, **:p<0.01, ***:p<0.005, ****:p<0.0001) as described in Example 2. FIG. 6A is a graph showing the relative fluorescence level of aptamer probes targeting H1, H3 and H7 respectively (Labels—A: aptamer; A+T: aptamer-target RNA pair; +ve: positive control with miniSpinach). None of the tested aptamer-target pairs for H1 gave a positive on/off ratio in a statistically significant manner, while one tested aptamer-target pair for H3 (target sequence is 757-778) and two tested aptamer-target pair for H7 (target sequences are 474-495 and 714-735) had statistically significant positive on/off ratio. FIG. 6B shows the results obtained for aptamer probes targeting N1, N2 and N9 (Labels—A: aptamer; A+T: aptamer-target RNA pair; +ve: positive control with miniSpinach). Two aptamers for N1 (target sequences are 86-107 and 862-883), one for N2 (target sequence is 694-715, orange), and two for N9 (target sequences are 369-390 and 545-566), had statistically significant positive on/off ratio. FIG. 6C show the results obtained for aptamers targeting PB2 (Labels—A: aptamer; A+T: aptamer-target RNA pair; +ve: positive control with miniSpinach). Two aptamers for PB2 (target sequences are 2209-2230 and 2247-2268) had statistically significant positive on/off ratio.

FIG. 7 is a heat map representation of the fluorescence signals measured by the microplate reader (N=3) of various aptamer-target pairs. The results indicated that the aptamer-target pairs are significantly orthogonal, meaning the aptamers are specific to their respective RNA targets.

FIGS. 8A and 8B are the results of sensitivity test for determining the limit of detection of two aptamer probes (N2-694 probe and N9-545 probe). 2 uM aptamer probe was added to each tube containing different concentrations of RNA targets and DFHBI (3,5-difluoro-4-hydroxybenzylidene imidazolinone); blank sample contained only buffer and DFHBI. Upper panel is a chart of fluorescent signals obtained from probe-target pairs of varying concentrations of the target RNA. Horizontal lines indicate the background fluorescent signals recorded from the aptamer by the plate reader plus 3 standard deviations. Lower panel is a photo taken by ChemiDoc Imager under SYBR Green mode with Blue Trans Light Excitation.

FIGS. 9A-9D show the results of ion dependence study of two aptamer probes (N2-694 probe and N9-545 probe). The arrows (↔) indicate the approximate concentration of ion present in the reaction mixture after the addition of nasal fluid (such concentration is referred to as “target ion concentration” herein). In FIG. 9A, both probes showed a good on-off ratio with the addition of sodium ion. In FIG. 9B, both probes demonstrated a general trend that the signals increased with concentration of potassium ion. The two probes showed a good on-off ratio in the range of target ion concentration. In FIG. 9C, both probes demonstrated a general trend that the signals dropped as the concentration of calcium ion increased. Nevertheless, in the range of target ionic concentration, both probes were able to yield significant on/off signals as analyzed by Two-Way ANOVA. In FIG. 9D, both probes showed a good on-off ratio with the addition of magnesium ion.

FIGS. 10A-10B show the change in fluorescent signals of two aptamer probes (N2-694 probe and N9-545 probe) in response to change in temperature in a real-time PCR study. FIG. 10A shows time and temperature for signal development of N9-694 and N2-545 probes (N=1). FIG. 10B shows the melting curves of N9-694 and N2-545 probes (N=1).

FIG. 11 is a flowchart schematically depicting the steps of data input, analysis and output in the methods and software representing some embodiments of the present invention.

FIG. 12 is a schematic representation of the information flow in some embodiments of the present invention.

FIG. 13 depicts a screening window along the target sequence used to design RNA aptamer probes in some embodiments of the present invention.

FIGS. 14A-14B graphically represent two scoring equations obtained by multiple linear regression analysis. FIG. 14A is a graph showing Score A values calculated according to some embodiments of the present invention plotted against the mean fluorescent counts of corresponding RNA aptamer probes. FIG. 14B is a graph showing Score I values calculated according to some embodiments of the present invention plotted against the on/off ratio as experimentally determined for the corresponding RNA aptamer probes.

FIGS. 15A-15B are graphs illustrating the relative fluorescence obtained from aptamer probes and RNA target synthesized in E. coli in a whole cell screening assay. In FIG. 15A, fluorescence was measured in E. coli BL21 (DE3) transformed with the constructs of aptamer probe, RNA target or both using a microplate reader (N=3) as described in Example 5. In FIG. 15B, RNA was extracted from the E. coli BL21 (DE3) after the whole cell screening assay. Fluorescence was measured using a microplate reader (N=1) and compared with other screening approaches (whole cell or in vitro transcription).

FIG. 16 is a photograph of a fluorometer device representing one embodiment of the present invention. Moveable lens handle 4 is attached to cover 6 attached to main body 7 to which a battery pack 10 is attached.

FIG. 17 is a photograph of a fluorometer device representing one embodiment of the present invention. The following parts are labeled: the first lens holder 1 housing the focusing lens; the second lens holder 2 housing the conversion lens 12; lens fixer 3; moveable lens handle 4; cover 6; main body 7; light-emitting diode 8; bandpass filter 9; mirror 11.

FIG. 18 is a schematic representation of a fluorometer device representing one embodiment of the present invention. Panel A shows the fluorometer with cover closed. Panel B shows the fluorometer with cover opened. Panel C is a top view if the fluorometer with cover removed. Panel D is a top view of the fluorometer with cover closed. Cover 6 is attached to main body 7 and a moveable lens handle 4 is attached to cover 6. Main body 7 houses the second lens holder 2 housing the conversion lens; the first lens holder 1 housing the focusing lens; lens fixer 3; filter holder 5.

FIG. 19 shows an electrical circuit design for a light emission system of a fluorometer device representing one embodiment of the present invention.

FIG. 20A shows a photograph of a light-emitting diode of a fluorometer device representing one embodiment of the present invention.

FIG. 20B is a schematic diagram of a light-emitting diode of a fluorometer device representing one embodiment of the present invention.

FIG. 21A is a schematic representation of a fluorometer device representing one embodiment of the present invention showing the location of the LED light source, the mirror, the sample holder, the bandpass filter as well as the first convex lens (conversion lens) and the second (moveable) convex lens (focusing lens).

FIG. 21B is a schematic representation of a fluorometer device representing one embodiment of the present invention demonstrating the pass of excitation light from the LED source. The light emitted by the LED source passes through the conversion lens, which converts the rays of light emitted by the LED source into parallel rays, the light is then reflected by the mirror, which changes its path by 90 degrees. The light then reaches the sample holder containing the biological sample and the RNA aptamer probes. Some of the fluorescence emitted by the probes passes through the focusing lens and then through the bandpass filter.

FIG. 21C is a schematic representation of light passing through the conversion lens which makes the rays of light parallel to each other.

FIG. 22 is a photograph of a star heat sink of a fluorometer device representing one embodiment of the present invention.

FIG. 23 depicts an LED current regulator of a fluorometer device representing one embodiment of the present invention.

FIG. 24 shows a diagram of a fluorometer device representing one embodiment of the present invention showing the outside dimensions of the device. All dimension are in mm.

FIG. 25 shows the dimension of the lid and the moveable lens holder of one embodiment of the present device in mm.

FIG. 26 shows the internal structure of a fluorometer device representing one embodiment of the present invention and the dimensions of the various parts in mm.

FIG. 27 shows the lens fixer of one embodiment of the present invention.

FIG. 28 shows the second lens holder for holding the conversion lens of one embodiment of the present invention. Light from the LED source passes through the conversion lens, which converts light rays to parallel.

FIG. 29 shows the lens moving handle which moves the focusing lens.

FIG. 30 shows the first lens holder for holding the focusing lens of one embodiment of the present invention. Fluorescent light from the sample passes through the focusing lens.

FIG. 31 shows a lid encapsulating the LED current regulator in one embodiment of the present invention.

FIG. 32 is a flowchart showing the functions of the image processing software in some embodiments of the present invention.

FIG. 33 is a design of the convolution neuro network of one embodiment of the present invention.

FIG. 34A is a 5×5 black and white phantom used to calibrate the distortion of the camera in some embodiments of the invention. FIG. 34B is a greyscale image of a 24-color phantom used for color correction in some embodiments of the present invention.

FIG. 35 is a schematic diagram of two constructs for co-expressing the RNA aptamer probe and target RNA in E. coli in a whole cell screening assay.

FIG. 36 shows relative fluorescence as measured by a microplate reader (upper panel) and Tracer representing one embodiment of the present invention (lower panel) as described in Example 7 and Table 12.

FIG. 37 depicts photographs of GFP samples prepared as described in Example 7 taken with an iPhone 6.

FIG. 38 is a flowchart illustrating deep neural network image processing module of some embodiments of the present invention.

FIG. 39 is the chemical structure of 3,5-difluoro-4-hydroxybenzylidene imidazolinone (DFHBI).

FIG. 40 shows the formation mechanism of the Spinach-DFHBI fluorescent complex.

FIG. 41 shows an embodiment of the aptamer design after undergoing the mechanism of FIG. 3.

FIG. 42 shows the oligo RNA synthesis for both aptamers and target sequence.

FIG. 43 shows the calculations of concentrations by nanodrop.

FIG. 44 is the data analysis workflow of features election and dimension reduction.

FIG. 45 shows the independent and dependent variables of non-structural features.

FIG. 46 shows an ideal design of the aptamer-target heterodimer.

FIG. 47 shows some false positive cases. The diagram on the left shows a p-p homodimer, and the diagram on the right shows a probe monomer.

FIGS. 48A-48T are the plot diagrams of fold change versus each non-structural parameter.

FIG. 49 is a visualization of the Normalized Mutual Information (NMI) results.

FIG. 50 shows a software flow chart.

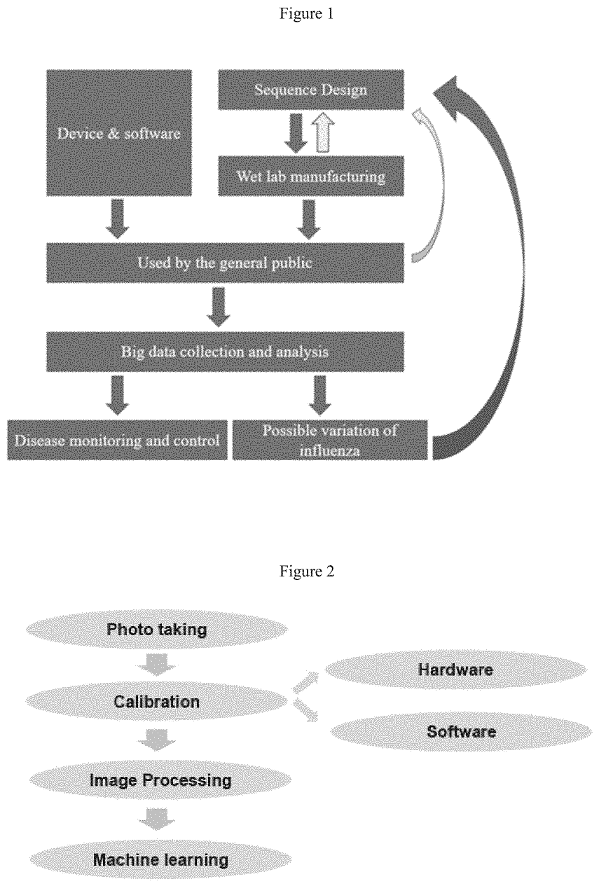

### DETAILED DESCRIPTION OF THE INVENTION

### RNA Aptamer Probes

In one embodiment, the present invention provides compositions of nucleic acids which are capable of binding to target nucleic acid sequences of a particular organism, such as influenza virus, and are capable of binding to a fluorophore molecule serving as a reporter. Fluorophore and fluorogen are used interchangeably in this description.

In one embodiment, the present invention provides compositions of RNA aptamer probes. In some embodiments, the present RNA aptamer probes comprise an aptamer structure, a sequence complementary to the target sequence and a fluorogen-binding site. In some embodiments, the present RNA aptamer probes can serve as an RNA aptamer probe which specifically binds to its target sequences (such as gene sequence of a certain type or subtype of influenza virus) upon which it is able to interact with a fluorogen and produce detectable fluorescent signals. In some embodiments, the present RNA aptamer probe produces no or negligible level of fluorescence in the absence of its respective target sequence. Upon binding to its target sequence, the probe changes conformation, which causes it to interact with a fluorogen molecule in a way that produces fluorescence or increases the level of fluorescence.

In some embodiments, the present RNA aptamer probes are modified from light-up RNA aptamers (LURAs). Light-up RNA aptamers are able to bind to fluorogens and have been developed for RNA detection (Bouhedda F, 2018). Spinach RNA aptamer and Broccoli RNA aptamer which conjugate with fluorogen DFHBI (3,5-difluoro-4-hydroxybenzylidene imidazolinone) are some of the examples of LURAs. However, the present RNA aptamer probes are not limited to those aptamers or any LURAs existing at the time of this invention. Other RNA aptamer structures which can be modified to recognize specific gene sequences and bind to fluorogens can be employed for generating the present RNA aptamer probes. By the same token, the present invention is not limited to DFHBI, other fluorogens or reporting molecules which work with the chosen aptamer structure can be used.

### Spinach RNA Aptamer

### Design Principle of RNA Aptamer Probes

The Spinach aptamer, along with its structural characteristics and photophysics, is well-characterized (Bouhedda F, 2018). According to the crystal structures of Spinach and iSpinach-D5 aptamers, the Spinach aptamer generally consists of two arms, P1 and P2, surrounding a G-quadruplex containing docking site of its fluorogen, DFHBI. While the docking site is indispensable for the formation of the Spinach-DFHBI complex, the lengths of the P2 arm have been shown to be less important by previous mutagenic studies to shorten the arms. By contrast, it was found that the P1 arm length has a dramatic effect on the fluorescence level and a single-base deletion can lead to the complete loss of fluorescence in E. coli.

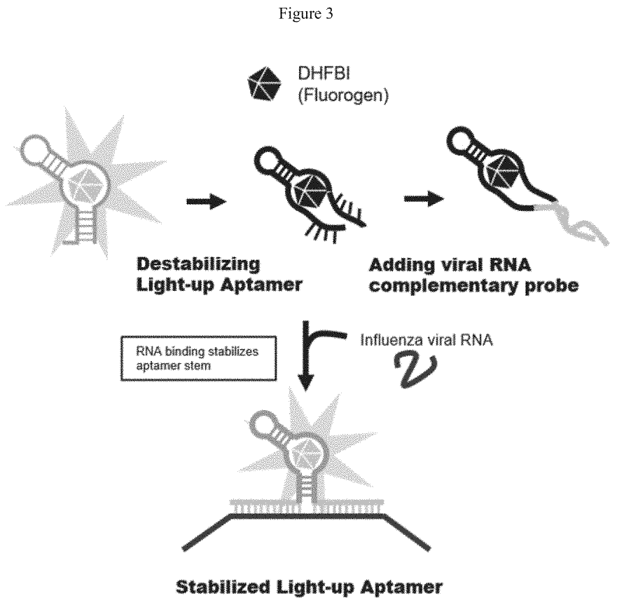

The present invention provides modular light-up RNA aptamers targeting influenza RNA. In one embodiment, the RNA aptamer is obtained by adding 11 base pair sequences complimentary to specific target viral RNA sequences to each side of the P1-truncated Spinach aptamer. The aptamer is modified by deleting one base pair at its stem, which functions as a stabilizer of the fluorescence-activating G-quadruplex structure. The modified Spinach aptamer has a misfolded or unfolded conformation when it is not bound to the target influenza RNA, and will change to a correct conformation when hybridizes to the RNA (FIG. 3).

### In Silico Design of RNA Aptamer Probes

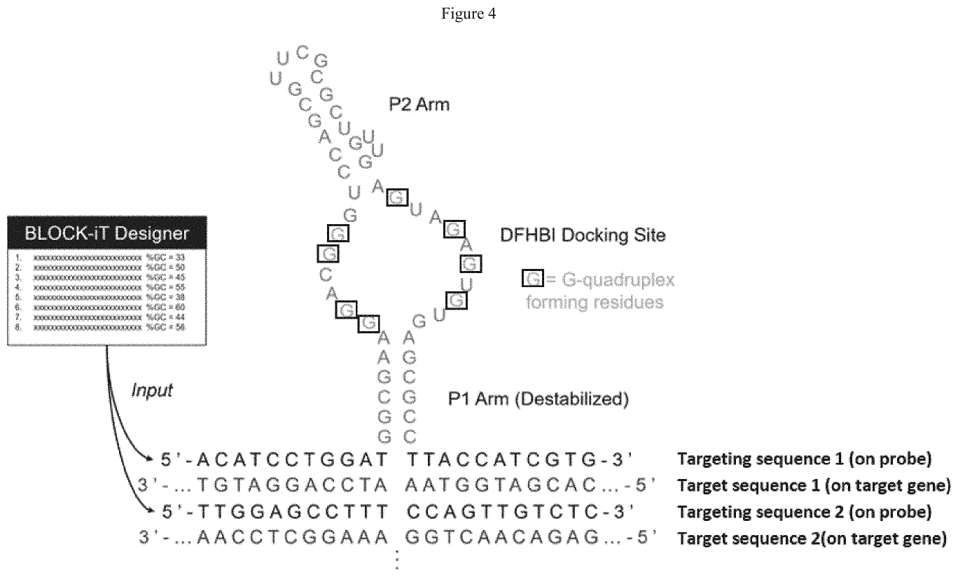

There were no known algorithms for predicting the binding of RNA to DFHBI and the resulting fluorescence level at the time of this invention. According to previous data, shortening of the P2 arm did not lead to a significant change in the fluorescence level of the Spinach aptamer (Ong, 2017). Thus, two presumptions were made in the present probe design process: (1) the formation of the DFHBI docking site is dependent on the correct folding of the P1 arm and (2) the P1 arm folding is optimized when the hybridization of the variable regions is the most favorable. Based on these two assumptions, RNA aptamer probes containing sequences complementary to a total of 22-bp gene sequences of influenza virus A were designed using Invitrogen BLOCK-iT siRNA Designer, which can find the region of RNA with the least amount of secondary structures, as well as human genome BLAST (FIG. 4). The BLOCK-iT siRNA Designer web page generates the sequences of 25 bp length.

Table I lists genes of influenza virus A and their accession numbers for design of RNA aptamer probes representing some embodiments of the present invention. The hemagglutinin genes (H1, H3 and H7) and neuraminidase genes (N1, N2 and N9) were selected for influenza subtyping, while the region of Polymerase Basic 2 gene (PB2) that is ubiquitous in most influenza A genomes was chosen for influenza detection. After inputting the selected sequences into BLOCK-iT designer, candidate sequences with GC content around ˜50% were chosen and a 22-bp region of each of the chosen candidate sequences was randomly selected for probe design (FIG. 4).

As shown in FIG. 4, the P1 arm of the RNA aptamer probe comprises two non-targeting sequences linked with the DFHBI docking site and two targeting sequences of 11-bp at its two end, each of the targeting sequences is complementary to the target sequence to be recognized by the probe.

TABLE 1
Genes of influenza virus A for probe design

| Gene | Gene Accession Number |
|---|---|
| H1 | EU021262.1 |
| H3 | NC_007366.1 |
| H7 | CY235363.1 |
| N1 | AJ518101.1 |
| N2 | NC_007368.1 |
| N9 | CY235364.1 |
| PB2 | By informatics (March et al. |
| | 2008, J of Virology) |

A total of 27 RNA aptamer probes were designed. All probes have the P1 and P2 arms and the docking site sequences as shown in Table 2 (refer also to FIG. 4). The full DNA sequences for obtaining the probes by transcription and their target gene regions are shown in Table 3. The full RNA sequences of the probes are shown in SEQ ID NOs:144-171.

TABLE 2
Sequences of P1 and P2 arms and the docking sites
of the RNA aptamer probes.

| P1 | Docking | P2 | Docking | P1 |
|---|---|---|---|---|
| SEQ ID | SEQ ID | SEQ ID NO: 3 | SEQ ID NO: 4 | SEQ ID |
| NO: 1 | NO: 2 | uccagcguucgc | aguagagugug | NO: 5 |
| ggcgaa | ggacggg | gcuguug | | agcgcc |

TABLE 3
Sequences of RNA aptamer probes and target RNAs

| | | | Target | Targeting |
|---|---|---|---|---|
| Name | Target | | sequences | sequence in P1 |
| of | gene and | DNA/RNA Full sequence | of the gene | arm of the probe |
| Probe | region | (5′-3′) | (3′-5′) | (5′-3′) |
| N1-86 | N1 gene | SEQ ID NO: 6 | SEQ ID NO: 34 | SEQ ID NO: 88 |
| | (86-107) | gtttggattgaggcgaaggacgggt | caaaccuaacu | guuuggauuga |
| | | ccagcgttcgcgagttgagtagagt | | |
| | | gtgagcgccgtgactagccc | | |
| | | SEQ ID NO: 144 | SEQ ID NO: 35 | SEQ ID NO: 89 |
| | | guuuggauugaggcgaaggacg | cacugaucggg | gugacuagccc |
| | | gguccagcguucgcgcuguuga | | |
| | | guagagugugagcgccgugacua | | |
| | | gccc | | |
| N1-167 | N1 gene | SEQ ID NO: 7 | SEQ ID NO: 36 | SEQ ID NO: 90 |
| | (167-188) | acatatgtgtgggacgaaggacgggt | uguauacacac | acauaugugug |
| | | ccagcgttcgcgctgttgagtagagt | | |
| | | gtgagcgccancacccagg | | |
| | | SEQ ID NO: 145 | SEQ ID NO: 37 | SEQ ID NO: 91 |
| | | acauaugugugggcgaaggacgg | uaagugggucc | auucacccagg |
| | | guccaacguucgcgcuguugag | | |
| | | uagagugugagcgccauucaccc | | |
| | | agg | | |
| N1-411 | N1 gene | SEQ ID NO: 8 | SEQ ID NO: 38 | SEQ ID NO: 92 |
| | (411-432) | ggtcccatttgggcgaaggacgggt | ccaggguaaac | ggucccauuug |
| | | ccagcgttcgcactattgagtagagt | | |
| | | gtgagcgccaatgtttgtca | | |
| | | SEQ ID NO: 146 | SEQ ID NO: 39 | SEQ ID NO: 93 |
| | | ggucccauuugggcgaaggacgg | uuacaaacagu | aauguuuguca |
| | | guccaacguucacgcuguugag | | |
| | | uagagugugagcgccaauguuu | | |
| | | guca | | |
| N1-862 | N1 gene | SEQ ID NO: 9 | SEQ ID NO: 40 | SEQ ID NO: 94 |
| | (862-883) | aaccatgccagggcgaaggacgg | uugguacgguc | aaccaugccag |
| | | gtccagcgttcgcgctgttgagtaga | | |
| | | gtgtgagcgccttgtccctgca | | |
| | | SEQ ID NO: 147 | SEQ ID NO: 41 | SEQ ID NO: 95 |
| | | aaccaugccagggcgaaggacgg | aacagggacgu | uugucccugca |
| | | guccaacguucacgcuauugag | | |
| | | uagagugugagcgccuugucccu | | |
| | | gca | | |
| N9-369 | N9 gene | SEQ ID NO: 10 | SEQ ID NO: 42 | SEQ ID NO: 96 |
| | (369-390) | agcatagaaccggcgaaggacgg | ucguaucuugg | agcauagaacc |
| | | gtccagcgttcacgctgttgagtaga | | |
| | | gtgtgagcgcctgcattcatct | | |
| | | SEQ ID NO: 148 | SEQ ID NO: 43 | SEQ ID NO: 97 |
| | | agcauagaaccggcaaaggacgg | acguaaguaga | ugcauucaucu |
| | | guccagcguucgcgcuguugag | | |
| | | uagagugugagcgccugcauuca | | |
| | | ucu | | |
| N9-531 | N9 gene | SEQ ID NO: 11 | SEQ ID NO: 44 | SEQ ID NO: 98 |
| | (531-552) | ccatcgtggcaggcgaaggacggg | gguagcaccgu | ccaucguggca |
| | | tccagcgttcgcgctgttgagtagag | | |
| | | tgtgagcgccactagtacttg | | |
| | | SEQ ID NO: 149 | SEQ ID NO: 45 | SEQ ID NO: 99 |
| | | ccaucguggcaggcgaaggacgg | ugaucaugaac | acuaguacuug |
| | | guccagcguucgcgcuguugag | | |
| | | uagagugugagcgccacuaguac | | |
| | | uug | | |
| N9-545 | N9 gene | SEQ ID NO: 12 | SEQ ID NO: 46 | SEQ ID NO: 100 |
| | (545-566) | acatcctggatggcgaaggacgggt | uguaggaccua | acauccuggau |
| | | ccagcgttcgcgctgttgagtagagt | | |
| | | tgagcgccttaccatcgtg | | |
| | | SEQ ID NO: 150 | SEQ ID NO: 47 | SEQ ID NO: 101 |
| | | acauccuggauggcgaaggacgg | aaugguaacac | uuaccaucgug |
| | | guccagcguucgcgcuguugag | | |
| | | uagagugugagcgccuuaccauc | | |
| | | gug | | |
| N9-868 | N9 gene | SEQ ID NO: 13 | SEQ ID NO: 48 | SEQ ID NO: 102 |
| | (868-889) | tgagccctgccggcgaaggacggg | acucgggccgg | ugagcccugcc |
| | | tccagcgttcgcgctgttgagtagag | | |
| | | tgtgagcgccaattgtccctg | | |
| | | SEQ ID NO: 151 | SEQ ID NO: 49 | SEQ ID NO: 103 |
| | | ugagcccugccggcgaaggacgg | uuaacagggac | aauugucccug |
| | | guccagcguucgcgcuguugag | | |
| | | uagagugugagcgccaauugucc | | |
| | | cug | | |
| N2-165 | N2 gene | SEQ ID NO: 14 | SEQ ID NO: 50 | SEQ ID NO: 104 |
| | (165-186) | gttggttcacaggcgaaggacgggt | caaccaagugu | guugguucaca |
| | | ccagcgttcgcgctgttgagtagagt | | |
| | | gtgagcgcccagcatcactt | | |
| | | SEQ ID NO: 152 | SEQ ID NO: 51 | SEQ ID NO: 105 |
| | | guugguucacaggcgaaggacgg | gucguagugaa | cagcaucacuu |
| | | guccagcguucgcgcuguugag | | |
| | | uagagugugagcgcccagcauca | | |
| | | cuu | | |
| N2-530 | N2 gene | SEQ ID NO: 15 | SEQ ID NO: 52 | SEQ ID NO: 106 |
| | (530-551) | atgctatgcacggcgaaggacgggt | uacgauacgug | augcuaugcac |
| | | ccagcgttcgcgctgttgagtagagt | | |
| | | gtgagcgccacttgcttggt | | |
| | | SEQ ID NO: 153 | SEQ ID NO: 53 | SEQ ID NO: 107 |
| | | augcuaugcacggcgaaggacgg | ugaacgaacca | acuuacuugau |
| | | guccagcguucgcgcuguugag | | |
| | | uagagugugagcgccacuugcuu | | |
| | | ggu | | |
| N2-694 | N2 gene | SEQ ID NO: 16 | SEQ ID NO: 54 | SEQ ID NO: 108 |
| | (694-715) | acaaacgcatt/gcgaaggacgggt | uguuugcguaa | acaaacgcauu |
| | | ccagcgttcgcgctgttgagtagagt | | |
| | | gtgagcgccctgactcctgg | | |
| | | SEQ ID NO: 154 | SEQ ID NO: 55 | SEQ ID NO: 109 |
| | | acaaacgcauuggcgaaggacgg | gacugaggacc | cugacuccugg |
| | | guccagcguucgcgcuguugag | | |
| | | uagagugugagcgcccugacucc | | |
| | | ugg | | |
| N2-894 | N2 gene | SEQ ID NO: 17 | SEQ ID NO: 56 | SEQ ID NO: 110 |
| | (894-915) | ttggagcctttggcgaaggacgggt | aaccucggaaa | uuggagccuuu |
| | | ccagcgttcgcgctgttgagtagagt | | |
| | | gtgagcgccccagttgtctc | | |
| | | SEQ ID NO: 155 | SEQ ID NO: 57 | SEQ ID NO: 111 |
| | | uuggagccuuuggcgaaggacg | gcagucgguau | cgucagccaua |
| | | gguccagcguucgcgcuguuga | | |
| | | guagagugugugagcgccccaguug | | |
| | | ucuc | | |
| H1-483 | H1 gene | SEQ ID NO: 18 | SEQ ID NO: 58 | SEQ ID NO: 112 |
| | (483-504) | cgtcagccataggcgaaggacggg | gcagucgguau | cgucagccaua |
| | | tccagcgttcgcgctgttgagtagag | | |
| | | tgtgagcaccgcaaatttttg | | |
| | | SEQ ID NO: 156 | SEQ ID NO: 59 | SEQ ID NO: 113 |
| | | cgucagccauaggcgaaggacgg | cguuuaaaaac | gcaaauuuuug |
| | | guccagcguucgcgcuguugag | | |
| | | uagagugugagcgccgcaaauuu | | |
| | | uug | | |
| H1-660 | H1 gene | SEQ ID NO: 19 | SEQ ID NO: 60 | SEQ ID NO: 114 |
| | (660-681) | ggtgaatttccggcgaaggacgggt | ccacuuaaagg | ggugaauuucc |
| | | ccagcgttcgcgctgttgagtagagt | | |
| | | gtgagcgcctgctataatgt | | |
| | | SEQ ID NO: 157 | SEQ ID NO: 61 | SEQ ID NO: 115 |
| | | ggugaauuuccggcgaaggacgg | acgauauuaca | ugcuauaaugu |
| | | guccagcguucgcgcuguugag | | |
| | | uagagugugagcgccugcuauaa | | |
| | | ugu | | |
| H1-825 | H1 gene | SEQ ID NO: 20 | SEQ ID NO: 62 | SEQ ID NO: 116 |
| | (825-846) | gatgattcctgggcgaaggacgggt | cuacuaaggac | gaugauuccug |
| | | ccagcgttcgcgagttgagtagagt | | |
| | | gtgagcgccatccaaagcct | | |
| | | SEQ ID NO: 158 | SEQ ID NO: 63 | SEQ ID NO: 117 |
| | | gaugauuccugggcgaaggacgg | uagguuucgga | auccaaagccu |
| | | guccagcguucgcgcuguugag | | |
| | | uagagugugagcgccauccaaag | | |
| | | ccu | | |
| H3-416 | H3 gene | SEQ ID NO: 21 | SEQ ID NO: 64 | SEQ ID NO: 118 |
| | (416-437) | aaactccagtgggcgaaggacggg | uuugaggucac | aaacuccagug |
| | | tccagcgttcgcgctgttgagtagag | | |
| | | tgtgagcgcctgccggatgag | | |
| | | SEQ ID NO: 159 | SEQ ID NO: 65 | SEQ ID NO: 119 |
| | | aaacuccagugggcgaaggacgg | acggccuacuc | ugccggaugag |
| | | guccagcguucgcgcuguugag | | |
| | | uagagugugagcgccugccggau | | |
| | | gag | | |
| H3-757 | H3 gene | SEQ ID NO: 22 | SEQ ID NO: 66 | SEQ ID NO: 120 |
| | (757-778) | caatagatgctggcgaaggacgggt | guuaucuacga | caauagaugcu |
| | | ccagcgttcgcgctgttgagtagagt | | |
| | | gtgagcgcctattctgctgg | | |
| | | SEQ ID NO: 160 | SEQ ID NO: 67 | SEQ ID NO: 121 |
| | | caauagaugcuggcgaaggacgg | auaagacgacc | uauucugcugg |
| | | guccagcguucgcgcuguugag | | |
| | | uagagugugagcgccuauucugc | | |
| | | ugg | | |
| H3-758 | H3 gene | SEQ ID NO: 23 | SEQ ID NO: 68 | SEQ ID NO: 122 |
| | (758-779) | ccaatagatgcggcgaaggacggg | gguuaucuacg | ccaauagaugc |
| | | tccagcgttcgcgctgttgagtagag | | |
| | | tgtgagcgccttattctgctg | | |
| | | SEQ ID NO: 161 | SEQ ID NO: 69 | SEQ ID NO: 123 |
| | | ccaauagaugcggcgaaggacag | aauaagacgac | uuauucugcug |
| | | guccagcguucgcgcuguugag | | |
| | | uagagugugagcgccuuauucu | | |
| | | gcug | | |
| H3-1305 | H3 gene | SEQ ID NO: 24 | SEQ ID NO: 70 | SEQ ID NO: 124 |
| | (1305-1326) | tagtgtcctcaggcgaaggacgggt | aucacaggagu | uaguguccuca |
| | | ccagcgttcgcgctgttgagtagagt | | |
| | | gtgagcgccacatatttctc | | |
| | | SEQ ID NO: 162 | SEQ ID NO: 71 | SEQ ID NO: 125 |
| | | uaguguccucaggcgaaggacgg | uguauaaagag | acauauuucuc |
| | | guccagcguucgcgcuguugag | | |
| | | uagagugugagcgccacauauuu | | |
| | | cuc | | |
| H7-474 | H7 gene | SEQ ID NO: 25 | SEQ ID NO: 72 | SEQ ID NO: 126 |
| | (474-495) | tgacaggagccggcgaaggacgg | acuguccucgg | ugacaggagcc |
| | | gtccagcgttcgcgctgagagtaga | | |
| | | gtgtgagcgccatttcatttct | | |
| | | SEQ ID NO: 163 | SEQ ID NO: 73 | SEQ ID NO: 127 |
| | | ugacaggagccggcgaaggacgg | uaaaguaaaga | auuucauuucu |
| | | guccagcguucgcgcuguugag | | |
| | | uagagugugagcgccauuucauu | | |
| | | ucu | | |
| H7-637 | H7 gene | SEQ ID NO: 26 | SEQ ID NO: 74 | SEQ ID NO: 128 |
| | (637-658) | attagaactccggcgaaggacgggt | uaaucuugagg | auuagaacucc |
| | | ccagcgttcgcgctgttgagtagagt | | |
| | | gtgagcgcccaactgtcacc | | |
| | | SEQ ID NO: 164 | SEQ ID NO: 75 | SEQ ID NO: 129 |
| | | auuagaacttccggcgaaggacgg | guugacagugg | caacugucacc |
| | | guccagcguucgcgcuguugag | | |
| | | uagagugugagcgcccaacuguc | | |
| | | acc | | |
| H7-714 | H7 gene | SEQ ID NO: 27 | SEQ ID NO: 76 | SEQ ID NO: 130 |
| | (714-735) | caatgaaagtcggcgaaggacggg | guuacuuucag | caaugaaaguc |
| | | tccagcgttcgcgctgttgagtagag | | |
| | | tgtgagcgccaattcttccgg | | |
| | | SEQ ID NO: 165 | SEQ ID NO: 77 | SEQ ID NO: 131 |
| | | caaugaaagucggcgaaggacgg | uuaagaaggec | aauucuuccgg |
| | | guccagcguucgcgcuguugag | | |
| | | uagagugugagcgccaauucuuc | | |
| | | cgg | | |
| H7-831 | H7 gene | SEQ ID NO: 28 | SEQ ID NO: 78 | SEQ ID NO: 132. |
| | (831-852) | acctgtacaccggcgaaggacggg | uggacaugugg | accuguacacc |
| | | tccagcgttcgcgctgttgagtagag | | |
| | | tgtgagcgccactctggattc | | |
| | | SEQ ID NO: 166 | SEQ ID NO: 79 | SEQ ID NO: 133 |
| | | accuguacaccggcgaaggacgg | ugagaccuaag | acucuggauuc |
| | | guccagcguucgcacuguugag | | |
| | | uagagugugagcgccacucugga | | |
| | | uuc | | |
| PB2- | PB2 gene | SEQ ID NO: 29 | SEQ ID NO: 80 | SEQ ID NO: 134 |
| 2209 | (2209- | ttaccaacaccggcgaaggacggg | aaugguugugg | uuaccaacacc |
| | 2230) | tccagcgttcgcgctgttgagtagag | | |
| | | tgtgagcgccacgtctccttg | | |
| | | SEQ ID NO: 167 | SEQ ID NO: 81 | SEQ ID NO: 135 |
| | | uuaccaacaccggcgaaggacgg | ugcagaggaac | acgucuccuug |
| | | guccagcguttcgcgcuguugag | | |
| | | uagagugugagcgccacgucucc | | |
| | | uug | | |
| PB2- | PB2 gene | SEQ ID NO: 30 | SEQ ID NO: 82 | SEQ ID NO: 136 |
| 2247 | (2247- | agagttccggcggcgaaggacggg | ucucaaaaccg | agaguuccggc |
| | 2268) | tccagcgttcgcgctgttgagtagag | | |
| | | tgtgagcgcctagagttccgt | | |
| | | SEQ ID NO: 168 | SEQ ID NO: 83 | SEQ ID NO: 137 |
| | | agaguuccggcggcgaaggacgg | aucucaaggca | uagaguuccgu |
| | | guccaacguucacgcuauugag | | |
| | | uagagugugagcgccuagaguuc | | |
| | | cgu | | |
| PB2- | PB2 gene | SEQ ID NO: 31 | SEQ ID NO: 84 | SEQ ID NO: 138 |
| 2265 | (2265- | gctgtcagtaaggcgaaggacggg | cgacagucauu | gcugucaguaa |
| | 2286) | tccagcgttcgcgctgttgagtagag | | |
| | | tgtgagcgccgtatgctagag | | |
| | | SEQ ID NO: 169 | SEQ ID NO: 85 | SEQ ID NO: 139 |
| | | gcugucaguaaagcgaaggacgg | cauacgaucuc | guaugcuagag |
| | | guccagcguucgcgcuguttgag | | |
| | | uagagugugagcgccguaugcua | | |
| | | gag | | |
| PB2- | PB2 gene | SEQ ID NO: 32 | SEQ ID NO: 86 | SEQ ID NO: 140 |
| 2300 | (2300- | cttttaattctggcgaaggacgggtc | aaaaauuaaga | cuuuuaauucu |
| | 2321) | cagcgttcgcgctgttgagtagagtg | | |
| | | tgagcgcctttggtcgctg | | |
| | | SEQ ID NO: 170 | SEQ ID NO: 87 | SEQ ID NO: 141 |
| | | cuuuuaauucuggcgaaggacgg | aaaccagcgac | uuuggucgcug |
| | | guccagcauucgcacuguugag | | |
| | | uagagugugagcgccuuugguc | | |
| | | gcug | | |
| mini- | Positive | SEQ ID NO: 33 | | |
| Spinach | control | gggagaaggacgggtccagcgttc | | |
| (P1-a5- | | gcgctgttgagtagagtgtgagctcc | | |
| b3) | | c | | |
| | | SEQ ID NO: 171 | | |
| | | gggagaaggacggguccagcguu | | |
| | | cgcgcuguugaguagaguguga | | |
| | | gcuccc | | |

### In Vitro Screening of Probes

### a) Production of RNA Aptamer Probes and Target RNA

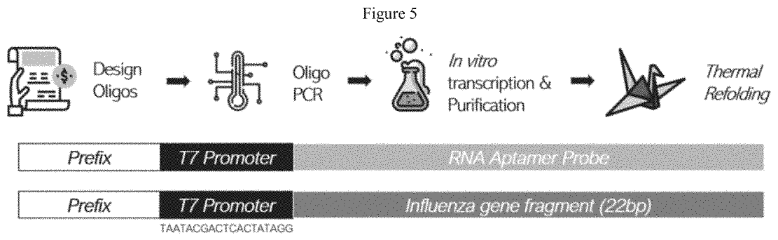

In vitro transcription kits were used to produce RNA probes and their target RNAs for assays. Example 1 and FIG. 5 describe procedures for the production of RNA aptamer probes and target RNA molecules representing some embodiments of the present invention using in vitro transcription kits. Other methodologies and kits that are capable of producing specific RNA sequences can also be used in connection with this invention.

### b) In Vitro Aptamer Refolding Essay

In order to investigate the effectiveness of the designed aptamers, their refolding ability upon binding to the target RNA sequences was tested according to the procedures described in Example 2.

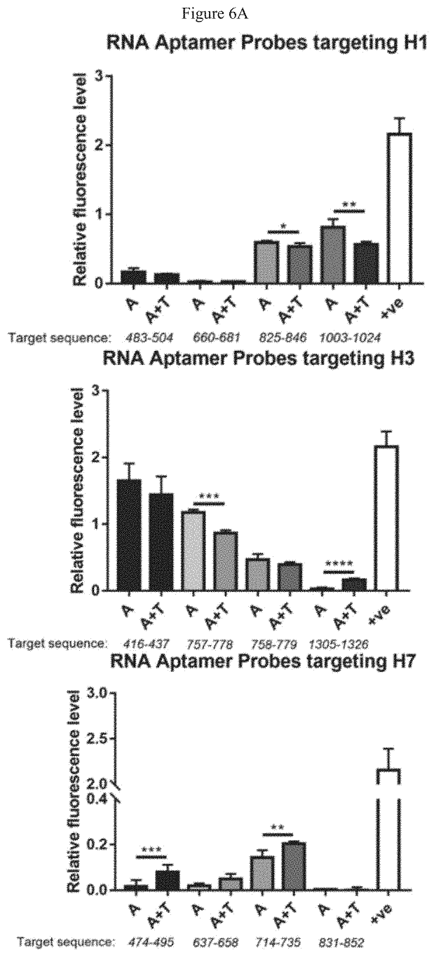

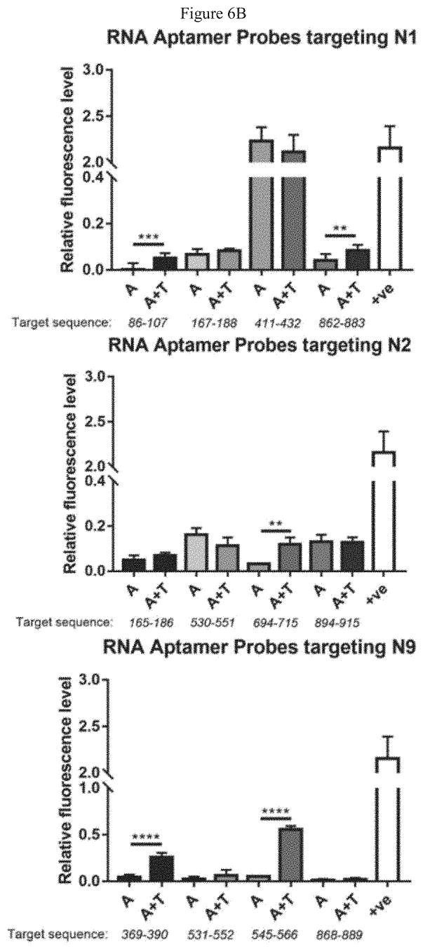

FIGS. 6A-6C show the results of refolding assay of aptamers designed with BLOCK-iT RNAi Designer for H1, H3, H7, N1, N2, N9, Polymerase Basic 2 (PB2) respectively. A total of four candidates of each type of aptamers was tested, the candidate should be able to refold to its correct confirmation upon binding to its RNA target and thereby giving a fluorescent signal in order to serve a detection purpose.

On/off ratio (i.e. the ratio of fluorescent signal produced in the presence of the target sequence to the fluorescent signal produced when target sequence is absent) is indicative of the ability of aptamer probe to detect its respective RNA target. If the intensity of fluorescence obtained from the aptamer-target RNA pair increases in a statistically significant manner as compared to the signals obtained from the aptamer alone (i.e., a statistically significant on/off ratio), the aptamer candidate are selected for further investigation. The results in FIGS. 6A-6C indicated that at least one candidate probe from each subtype yielded a statistically significant on/off ratio as determined by the student's t-test, except for probes specific to H1.

### Characterization of RNA Aptamers

### a) Specificity

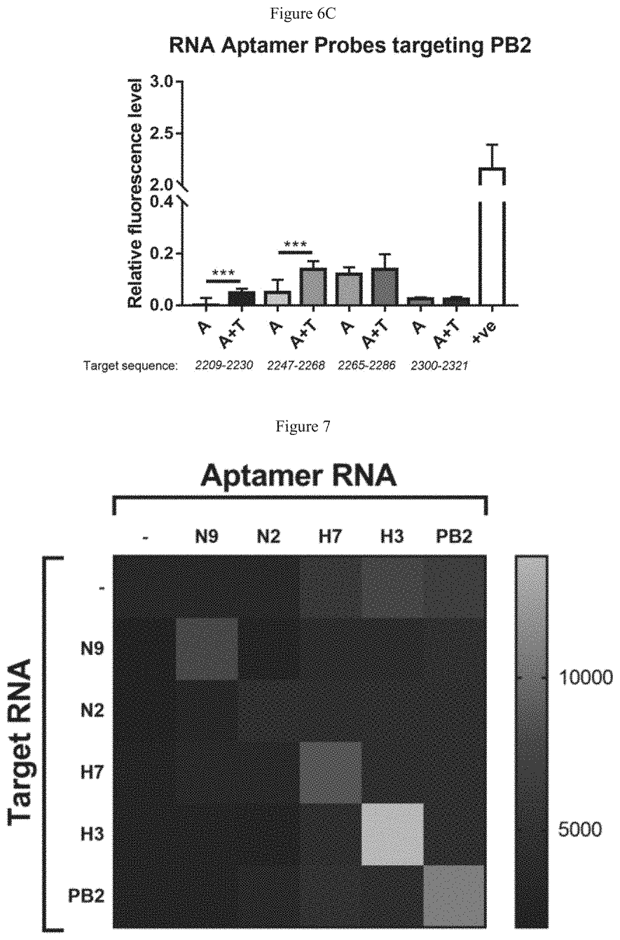

Though the present aptamers were designed to target hemagglutinin or neuraminidase genes of specific influenza subtypes, unwanted binding between the aptamers specific to a particular subtype and sequences from non-target subtype(s) may occur. Five of the tested aptamers (i.e. for N9, N2, H7, H3, PB2) that performed well in the above mentioned refolding assay were selected to investigate their cross-reactivity. Using the procedures for refolding assay described in Example 2, the aptamers were mixed with -their target or non-target sequences and the resulting fluorescence were measured. An aptamer is regarded to be specific if it gives statistically significant on-off signal in response to its target RNA but not non-target RNA. FIG. 7 is a heat map representation of the resulting microplate reader data (N=3), the results indicated that the aptamer-target pairs are significantly orthogonal. Two-Way ANOVA was used to analyze the data and the results indicate that signals obtained from aptamer-target pairs (“interaction”) were changed the most (Table 4).

TABLE 4
Two-Way ANOVA analysis of signals obtained from
the aptamer, target RNA and aptamer-target pair

| Source of variation | % of total variation | P-value |
|---|---|---|
| Interaction | 61.9 | <0.0001 |
| Target RNA | 3.183 | <0.0001 |
| Aptamer RNA | 33.69 | <0.0001 |

### b) Detection Limit (Sensitivity)

It is important to ascertain the minimum amount of target viral RNA needed to distinguish between the fluorescent signals from negative and positive samples under the blue light box by naked eye, in order to understand at which stage of influenza latency viral RNA could be detected by visual examination. Generally, the detection limit of an aptamer probe depends on the level of background noise generated by the aptamer.

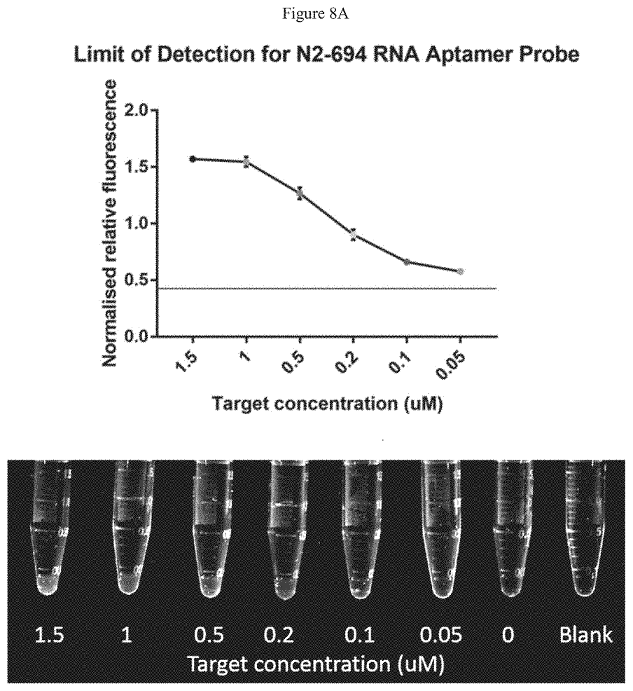

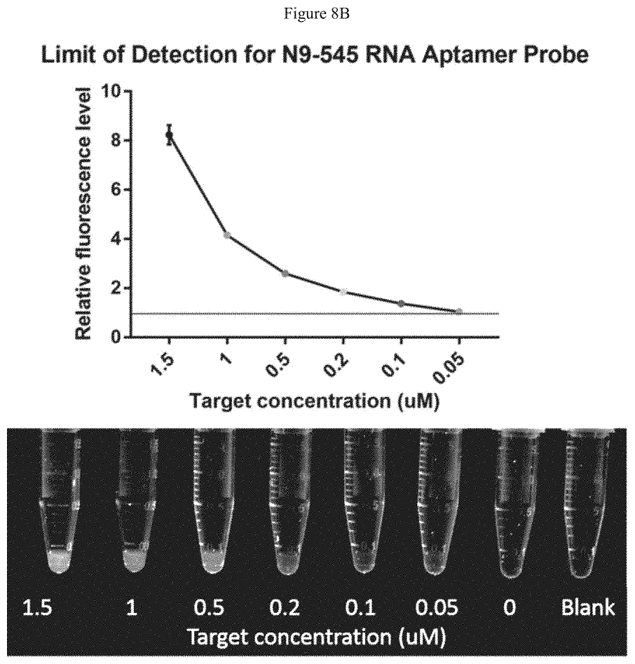

Example 3 describes the procedures for determining the minimum amount of target RNA required to obtain a visually distinguishable difference between positive and negative signals using two aptamer probes, N2-694 and N9-545, which represent a probe with a lower sensitivity and a probe with more background fluorescence respectively. Limit of detection can be determined by visual examination by naked eye which is less accurate, or by taking the value of the minimum amount of target RNA required to generate a signal that is larger than the signal generated by the aptamer only (i.e. negative signal) plus 3 standard deviations (the threshold value). In the upper panel of FIGS. 8A and 8B, the horizontal lines indicate the background fluorescent signals recorded from the aptamer by the plate reader plus 3 standard deviations (the threshold value). A target RNA can be detected by the present assay if its amount is capable of generating fluorescent signals larger than the threshold value.

FIGS. 8A and 8B show the results of N2-694 probe and N9-545 probe respectively obtained in the sensitivity study. The upper panel is a bar chart of fluorescent signals measured by CLARIOstar plate reader with Ex/Em 447/501, and the horizontal line indicates the threshold value of fluorescent signal. Fluorescent signal exceeding the threshold value indicates the presence of the target RNA. The lower panel is a photo taken by ChemiDoc Imager under SYBR Green mode with Blue Trans Light Excitation.

Visually, for N2-694 probe, more than 0.2 μM of target RNA was needed to visualize the difference, while about 0.2-0.5 μM of target RNA was required for N9-545 probe (See lower panels in FIGS. 8A and 8B).

### c) Ion Dependency

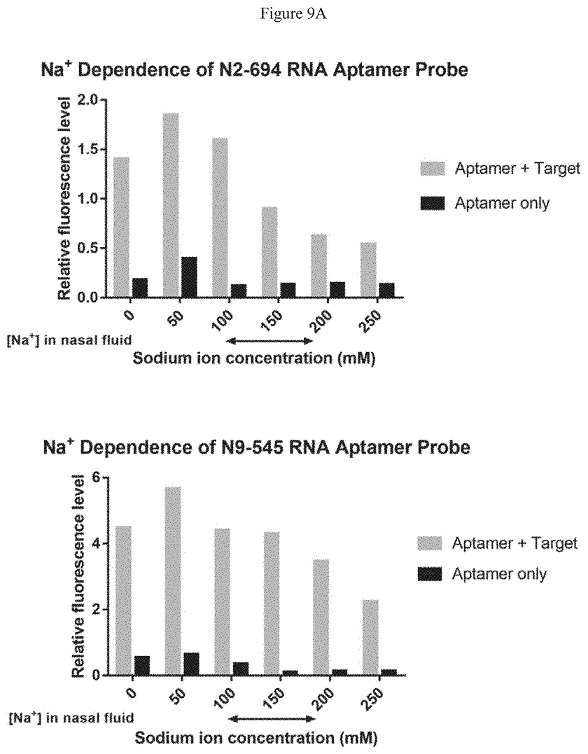

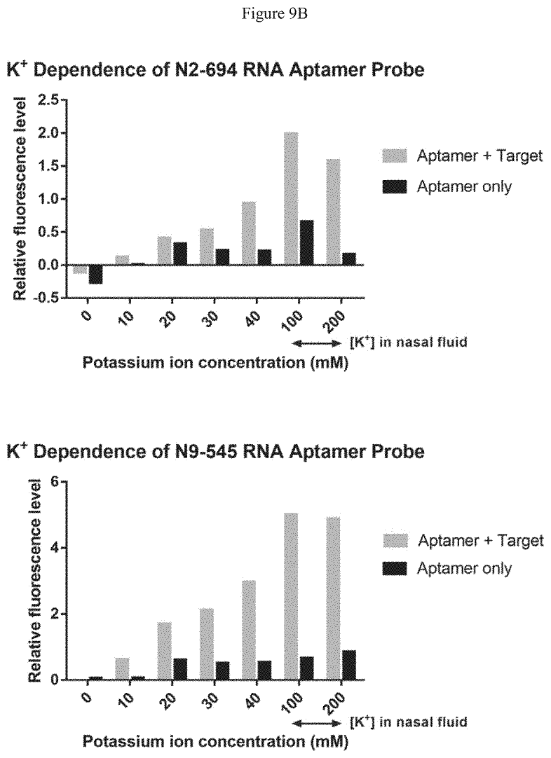

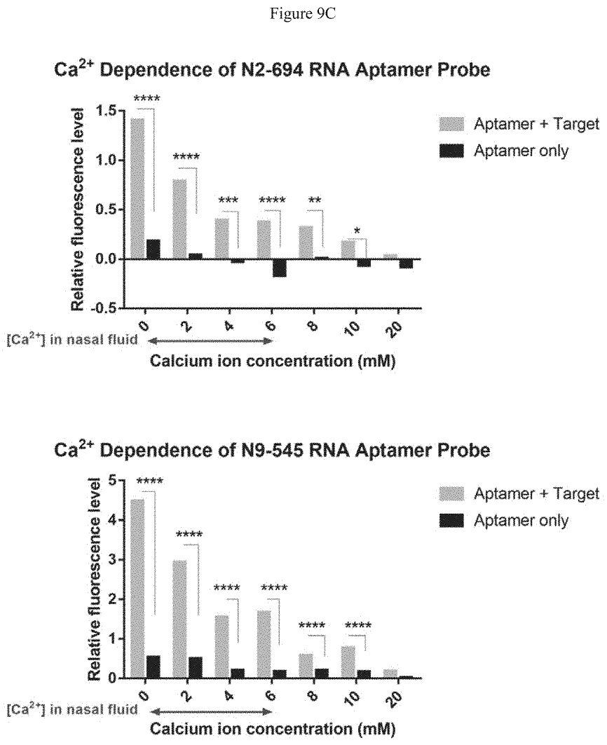

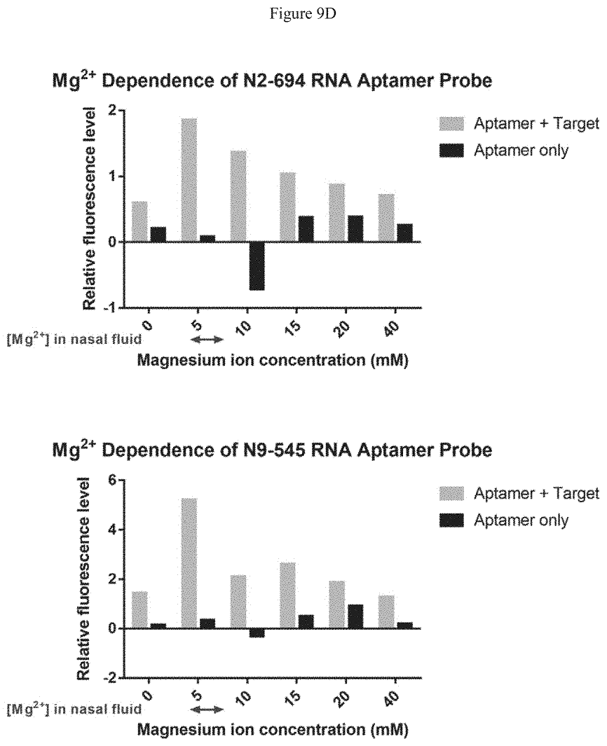

As in some embodiments of the invention, the test sample is nasal fluid obtained from individual subjects, performance of the RNA aptamers in the presence of nasal fluid was also evaluated to fully assess the detecting ability of the present RNA aptamer probes. In particular, performance of RNA aptamers in various ionic conditions (sodium, potassium, calcium and magnesium ions) in nasal fluid was tested as described in Example 4. The results are shown in FIGS. 9A through 9D.

As an example, N2-694 and N9-545 aptamers were tested. Different concentration of sodium, potassium, calcium and magnesium ions were added to the aptamer folding reaction mixture of N2-694 and N9-545 probes, mimicking the addition of nasal fluid to the freeze-dried aptamer kit by household users. Results obtained indicated that the two aptamer probes behaved similarly at different ion concentrations. In the range of target ionic concentrations (i.e., the ionic concentrations after addition of the nasal fluid which mimics the real situation in which the present invention is used; namely, 138-139 mM of sodium ion, 131-140 mM of potassium ion, 1-1.85 mM of calcium ion and 5.47-5.17 mM of magnesium ion, see Tables 9 and 10), the two probes performed well in elevated concentrations of sodium, potassium and magnesium ions, but an increase in calcium ion concentration resulted in a decrease of fluorescence signal of both probes. Both probes were able to give good on/off ratios in the range of target ionic concentrations and hence are suitable candidate for the purpose of on-site detection of influenza virus A.

Overall, the results indicated that the present aptamer system is not adversely affected by sodium, potassium and magnesium ions naturally present in the nasal fluid and is slightly affected by the elevated concentration of calcium ion. It is likely that the present aptamers would perform satisfactorily in term of detection of target RNA molecules when real samples instead of folding assay buffer are used.

### d) Optimization of Aptamer Folding Condition

Real-time PCT system was used to monitor the change in fluorescent signals and the time required for signal development.

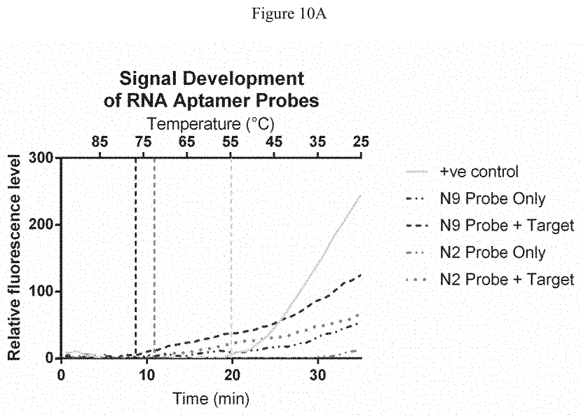

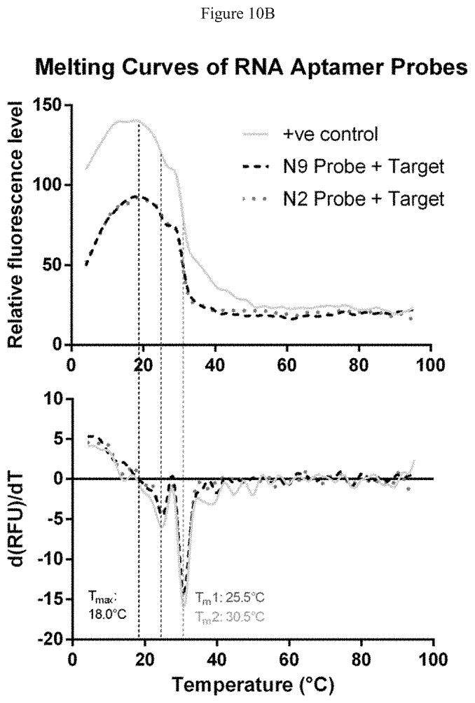

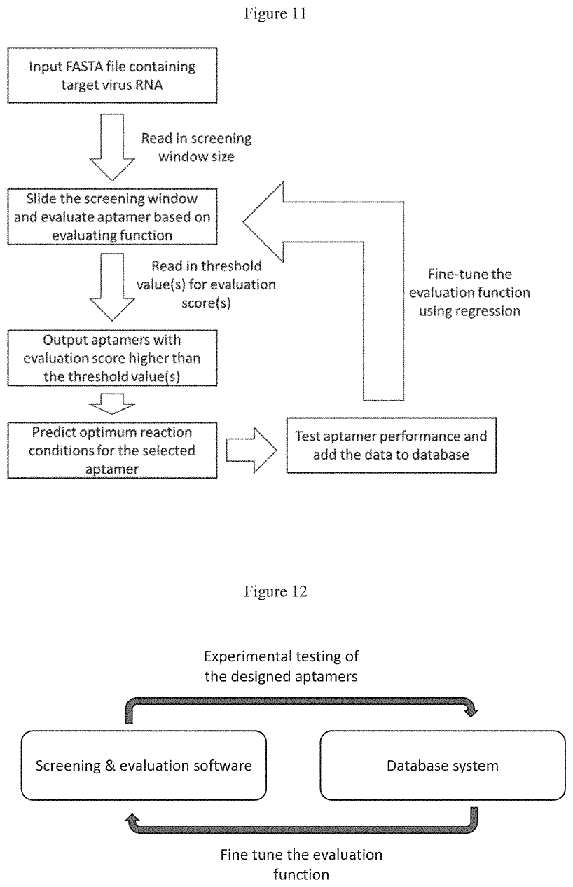

Using N9-694 and N2-545 as the candidate probes, it was shown that the probe-target pairs required about 10 minutes of cooling to give a detectable signal, and the temperature at that point was around 75° C. (FIG. 10A).

After refolding is completed, melting curve (dissociation) analysis of the two pairs of probe-target was performed with the same real-time PCR system (FIG. 10B). Surprisingly, the melting curve showed that the fluorescence intensity dropped quickly at 18-35° C., which does not correspond to the previous data (Strack, 2013). However, this might be caused by the asymmetry of association and dissociation kinetics of DFHBI docking, which was not described in the previous literature. Another interesting point is that both miniSpinach and probe-target pairs share two melting temperatures, suggesting a two-step mechanism of DFHBI docking. Nevertheless, the temperature where the probe achieved optimal performance is around 18° C. (Tₘₐₓ), while room temperature (about 25° C.) incubation can also achieve a very good fluorescence signal, meaning that the present aptamer probes can be used to detect target RNA and influenza virus under ambient conditions.

### Methods for Designing Aptamer Probes and Software Implementing the Methods

As mentioned above, the aptamers being tested in the present invention were designed from randomly selected sequences. The present invention further provides methods for rational design of RNA aptamer probes which may allow to design more effective RNA aptamers (e.g. aptamer with a higher ON/OFF ratio). An automated system for rational design of aptamers can also be built to enable a high-throughput design (i.e., design of a large number of aptamer probes specific to various target sequences quickly).

By comparing candidate aptamers which gave good and poor performance in the preceding studies, some methods of the present invention identify parameter(s) which may be adjusted and optimized to achieve a higher on/off ratio.

RNA aptamer probes which do not fluoresce in the absence of the target sequences but fluoresce upon binding to their target sequences and interacting with a fluorogen are desirable for the present purpose. That is, the RNA aptamer should have no or low fluorescence when not bound not its target sequence (also referred to herein as autofluorescence) and a high target-induced fluorescence. It is presumed that an RNA aptamer will have a low autofluorescence and a high induced fluorescence if:
1. The truncated P1 arm (miniSpinach) is destabilized in the aptamer so it is not be able to form the G-quadruplex structure responsible for fluorescence in the absence of the target sequence (resulting in no or low autofluorescence).
2. The destabilized truncated aptamer can bind to its target RNA and upon biding, the destabilized structure is “re-stabilized” which subsequently leads to fluorescence (i.e., target-induced fluorescence which is induced by target RNA).

Hence, for the purpose of rational design, the following data obtained from the preceding experiments were compared to evaluate the degree of auto-fluorescence and target-induced fluorescence of various aptamers:
1. fluorescence intensity when only the RNA aptamer probe and a fluorogen are present (auto-fluorescence);
2. fluorescence intensity when the target sequence if present in the sample. The ratio of #2 to #1 is the on/off ratio.

Generally, aptamers are designed using the method known as Systematic evolution of ligands by exponential enrichment (SELEX). However, SELEX is not cost-efficient, has a long development cycle and may not provide the optimal design. Currently, there is no known software that can be used to design RNA aptamers directly.

One embodiment of the present invention provides a method to screen and evaluate aptamers. It can serve as a tool to optimally design RNA aptamers.

One embodiment of the present invention provides a software implementing the method of screening and evaluating aptamers. The output of this software is cross validated with the results of experimental tests of the designed aptamers. Parameters used for designing the aptamers may be continuously fine-tuned based on experimental results by using regression analysis. Thus, as this system is used and more experimental data is added into the system, prediction of the optimal aptamer design by the software will become more and more accurate. Steps of this method are schematically depicted in Figures I1 and 12.

The screening and evaluation module and the database system are the two core components of the software. The screening and evaluation module evaluates candidate aptamer designs based on several factors. In one embodiment of the present invention, an on/off ratio is used as a measure of performance of aptamer probes. The on/off ratio is the ratio of fluorescent signal produced by a probe and a fluorogen in the presence of its target sequence to fluorescent signal produced when its target sequence is absent. Application of the method to design of miniSpinach aptamer probes targeting influenza virus RNA is described below as an example. The algorithm of the present method, or similar algorithms, can be applied to other types of aptamers and targeting sequences. Multiple linear regression analysis is used to model the relationship between the selected parameters and the performance of aptamer probes. Other types of regression analysis, such as polynomial regression, logarithmic regression and others may be used in various embodiments of the present invention.

### Screening & Evaluation Module

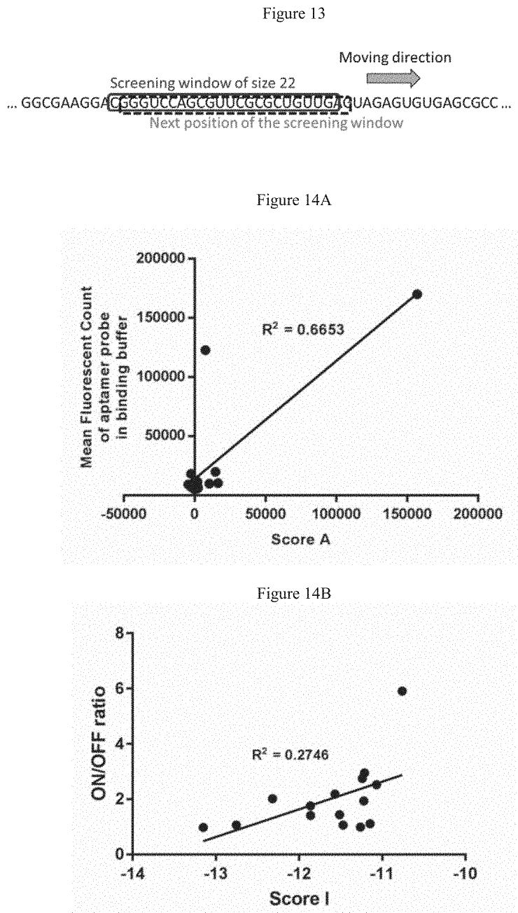

This module screens and evaluates candidate aptamer design. After a .fasta file containing the viral RNA sequence and a range of window sizes is entered into the software, a window slides from the first position to the end of the whole virus sequence as illustrated in FIG. 13. Then the module takes the sequence inside the window as a candidate for aptamer design, evaluates the aptamer based on several factors (listed below) and calculates a cumulative score (such as Score I described below). After that, the window moves to the right by one nucleotide. After evaluating all possible designs, the program outputs the top 5 designs. The stem and loop part of the aptamer shown in lighter font in FIG. 4 are the standard parts of the miniSpinach aptamer and the probe targeting sequences were designed using this method.

### Degree of Auto-Fluorescence

This part elucidated the correlation between the degree of destabilization of the truncated miniSpinach and the degree of auto-fluorescence of the destabilized aptamer in the presence of a fluorogen. In particular, the correlation between probability of binding between certain base pairs of the aptamer which may be responsible for stabilizing the aptamer structure and fluorescence obtained from the aptamer alone were evaluated. The correlation, if robustly established, may be used to determine whether an aptamer design likely gives rise to auto-fluorescence and thus is not suitable for making the present RNA aptamer probes.

Binding probability between certain pairs in an aptamer is an important indicator of whether the truncated miniSpanich is destabilized. Candidate aptamers are derived from a truncated miniSpanich (P1-a4-b5), which is produced by removing one base pair in the stem of the original fluorescing miniSpanich (P1-a5-b5) (described in Ong, 2017) reducing the number of base pairs in the stem from 5 to 4. Therefore, it is expected that a “well-destabilized” aptamer probe (one that does not autofluoresce) is less likely to have strong interactions between the remaining 4 base pairs in stem a, i.e., interactions between nucleotides 14-62, 15-61, 16-60 and 17-59 in a candidate aptamer probe. A scoring equation is determined by plotting the experimentally determined mean fluorescent count of the aptamers in the absence of target (N=17) against the binding probability between nucleotides 14-62, 15-61, 16-60 and 17-59 calculated by CentroidFold [2] and by multiple linear regression.

The best fit (largest R²) scoring equation for this dataset is:

Mean fluorescent Count=Constant+Score A+Error,
where Score A represents the sum of linear terms in regression analysis, given by Score A=−65075374 (binding probability between nucleotides 14 and 62)+706797383 (binding probability between nucleotides 15 and 61)−1617284819 (binding probability between nucleotides 16 and 60)+27305386 (binding probability between nucleotides 17 and 59)

A high Score A suggests the aptamer design is more likely to be auto-fluorescing. FIG. 14A shows a graph where the experimentally determined mean fluorescent count of aptamer probe in the absence of the target sequence is plotted against the Score A calculated based on the equation above.

As FIG. 14A illustrates, Score A shows a positive correlation with the mean fluorescent count of the aptamers. A high Score A suggests the aptamer design is more likely to be auto-fluorescing. The graph shows a particularly high score for one of the aptamer probes showing strong auto-fluorescence (the right-most point) but another strongly auto-fluorescing probe had a low A score. Given a relatively low R² value, this proposed scoring model has to be further tested with more data sets covering designs with high score and low score. If the present model is proven to be robust, it is expect that this model can be used for screening aptamer designs which are likely to be auto-fluorescing (having high Score A).

### Minimum Free Energy

Having a destabilized miniSpinach with no or low auto-fluorescence is insufficient as the destabilized miniSpinach is not necessarily inducible by the target RNA and hence may not exhibit target-induced fluorescence. Therefore, it is desirable to have another score for identifying aptamer designs that are more likely to be inducible by their target RNA through the analysis of the on/off ratio obtained as described in Example 2.

The effect of free energy on the performance of aptamer (as measured by the on/off ratio) is well supported by experimental data. Assuming that the formation of heterodimer between the aptamer and target is in equilibrium, and considering that the free energy (Delta G value) is related to thermodynamic stability, the following factors are selected for the regression analysis:
the minimal free energy (MFE) of aptamer-target heterodimer,
the MFE of aptamer-aptamer monomer,
the MFE of target-target homodimer,
the value of delta G for heterodimer binding, and
frequency of the MFE structure in the aptamer-target heterodimer.

The ON/OFF ratio is plotted against all factors above, and the best fit (largest R²) equation is then determined using multiple linear regression. Frequency of the MFE structure in the aptamer-target heterodimer included in the analysis may be related to the structural stability of the aptamer-target heterodimer and the structural dynamics of the assembled heterodimer.

The best fit equation for the data set studied (15 probes) is found to be:

On/Off Ratio=Constant+Score I+Error,

where Score I is the sum of linear terms in the regression analysis, given by
Score I=3.71 (MFE of aptamer monomer)+3.37 (MFE of target monomer)−3.42 (MFE of aptamer-target heterodimer)+3.61 (Delta G for heterodimer binding)+5.24 (The frequency of the MFE structure in the ensemble).
   Coefficients in the Score I equation above may change as more experimental data is added to the dataset on which the equation is based.

Score I is an indicator of the probability of formation of aptamer-target heterodimer. Higher Score I indicates higher probability of formation of aptamer-target heterodimer. FIG. 14B is a graph showing the experimentally determined on/off ratio plotted against score I as calculated according to the equation above.

### Target-Induced Fluorescence

This part elucidated the correlation between binding affinity between the destabilized miniSpinach and its target RNA and fluorescence of the aptamer-target pair. This correlation, if robustly established, may be used to determine whether a destabilized miniSpinach can be re-stabilized by target RNA thereby giving rise to target-induced fluorescence and hence is suitable for the making the present RNA aptamer probes.

As FIG. 14B illustrates, Score I showed a positive correlation with the on/off ratio, indicating that aptamer designs having a high Score I are more likely to be inducible. However, there are a few points which depart from the predicted trend and the R2 value only reaches 0.2746.

Since the regression only considered variables included in the equation which only concern the binding between the RNA molecules but not the docking of DFHBI, it is not surprising that the R² value is relatively low. While in the “turn-on” event, only representative variables for molecular dynamics between aptamer and target, as well as representative variables for structural dynamics (frequency of the MFE structure in the complex) were included, there is no representative variables for the molecular dynamics that account for the binding event of the heterodimer with DFHBI due to difficulties in predicting the interaction between an RNA and a small molecule (which is not an RNA).

In sum, although the R² values for both scoring methods are not high, they are still far from random. Therefore, it is reasonable to conclude that the two scoring models have the potential to assist a rational design for miniSpinach aptamer that is more likely to give a significant on/off signal inducible by its target RNA sequence, and also facilitate an automated, high-throughput screening of aptamer designs for targeting short or long target RNA sequences.

### Secondary Structures

Secondary structures play a key role in determining the expected performance of an aptamer. In the present method, secondary structures of candidate aptamers are predicted using the Vienna RNA python package. After prediction, two outcomes are considered:
a. If the predicted secondary structure indicates fluorescence without target sequences, the aptamer design is discarded;
b. If the predicted secondary structure does not indicate auto-florescence, other factors (such as the MFE, the AU/CG ratio described below, etc.) are considered.

### AU/CG Ratio

The ratio of different base pairs seems to influence the binding stability of RNA.

### Melting Temperature

The melting temperature of RNA refers to the temperature at which it is in single strand. Since the RNA aptamer has to be in single strand in order to interact with the target sequence and DFHBI, the melting temperature is also critical to the performance of RNA aptamer probes.

### Database System

Experimental performance data is generated for aptamer probes designed according to the present method and may be entered into the database system. Database may contain the following information:

Scores.

This is the output of the evaluation function, e.g. Score I and Score A.

Autofluorescence level and fluorescence level in the presence of the target sequence.

This is the direct reading of fluorescence level before and after adding target sequences. The absolute fluorescence value may vary in different settings, depending, for example, on the measurement setting and equipment used.

Fold change of fluorescence level.

The measured absolute fluorescence value may vary in different settings, depending, for example, on the measurement settings and equipment used. Thus, fold-change of fluorescent level may be used as a meaningful parameter. Fold change is a ratio of the fluorescent level in the presence of the target sequence to the fluorescent level in the absence of the target sequence.

Optimal detection environment.

The optimal detection environment may include such factors as ion composition of the sample, temperature, and concentrations of various sample components. Some embodiments of the present invention may be able to predict the optimal detection environment based on the experimental data.

The performance of the present methods may be evaluated by experimentally testing various aptamers designed using the method. The experimental data may be used to further refine and improve the methods.

In some embodiments, the screening step can comprise the steps of:
1. generating all possible aptamer designs by sliding window of 22 nucleotides on an RNA sequence that determines the variable domain of the miniSpinach aptamer probe,
2. eliminating designs that are likely to be auto-fluorescing, and
3. selecting designs that are likely to be inducible by the target RNA sequences.

In one embodiment, the present invention provides a method for designing a sequence of an RNA aptamer capable of binding to a target nucleic acid, the RNA aptamer comprises a G-quadruplex structure that is capable of binding to a fluorogen. In one embodiment, the method comprises:
(a) selecting a target nucleic acid sequence;
(b) generating a plurality of sequences of an oligonucleotide having a hybridizing sequence complementary to the target nucleic acid sequence;
(c) determining the binding probability between the generated sequence and nucleotides involved in the formation or stabilization of the G-quadruplex structure, thereby determining the likelihood of producing fluorescence in the absence of the target nucleic acid by the designed sequence;
(d) determining the minimal free energy of one or more of: (i) the heterodimer of aptamer-target nucleic acid, (ii) the homodimer of aptamer, (iii) the homodimer of target nucleic acid, and the frequency of the aforementioned heterodimer and homodimer, thereby determining the likelihood of giving a fluorescence upon binding to the target nucleic acid by the generated sequence; and
(e) designing the sequence of RNA aptamer according to the results of steps (c) and (d).

In one embodiment, the present method or system for designing a sequence of a RNA aptamer capable of binding to a target nucleic acid is implemented in combination of other methods or systems such as those available in the Vienna RNA secondary structure server (L. Ivo, 2003.)

### Production of RNA Aptamers and RNA Targets in Bacterial Cells and Whole-Cell Screening

In one embodiment, the present invention provides a method and system for producing RNA aptamers using bacterial expression system.

Bacterial expression systems are generally less costly than in vitro cell-free transcription kits, thus the present RNA aptamer probes and tests can be made more affordable to the public if the probes can be massively produced by a bacterial expression system. Research and development costs can also be reduced since the processes of screening, characterization and optimization usually require a considerable amount of probes and targets.

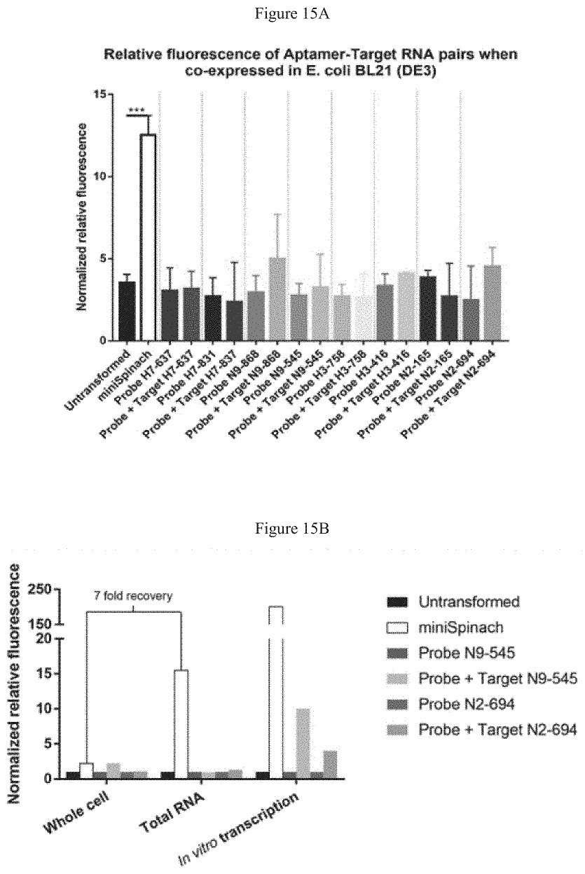

To explore the possibility of producing and screening RNA aptamer probes using bacterial system, RNA aptamer probes and their RNA targets were co-transformed and their interaction was evaluated by a whole-cell assay described in Example 5. The expected on/off ratio as observed in the in vitro cell-free refolding experiments was not observed in the whole cell assay (FIG. 15A). It might be due to the low abundance of the probes and the RNA targets in E. coli, as low fluorescence was also observed in the positive control (miniSpinach).

To verify, total RNA was extracted from the E. coli obtained after the whole-cell assay and an amount of RNA equivalent to the amount of RNA extracted from the same number of cells as was used in the whole cell assay was tested for its fluorescence level. Surprisingly, a 7-fold recovery of the fluorescence level of the positive control (miniSpinach) was observed in total RNA, while no recovery was observed in the probe-target pairs (FIG. 15B). Based on the results, it is concluded that while aptamer folding without tRNA (transfer RNA) scaffold is unfavorable in E. coli, probe-target hybridization is inhibited by the T7 terminator following the aptamer.

### Devices

In one embodiment, the present invention provides a battery-operated and mobile-phone-based device for detecting and processing light or fluorescent signals given out by the present RNA aptamer probes or other light-emitting moieties which indicate the presence of target organisms or their genetic materials.

At the time of this invention, there is no comparable mobile-phone and light based device for detection of influenza viruses. Current medical devices for influenza detection are costly and usually require expertise to operate, hence they are not convenient to use by the general public and the fees charged for clinical tests are high. As compared to currently available devices, the present device has an improved light path design which is accomplished by changing the light path by 90-degree to reduce background noises from excitation light rays and using a convex lens to convert the excitation light rays to parallel rays to avoid capturing undesirable light by the mobile camera. This lens may be referred to herein as conversion lens.

Apart from detection based on RNA aptamer probes (RAPID), some embodiments of the present invention can be used for color detection, fluorescent detection using other types of probes and emissive light detection. This may be achieved by changing the light source in the fluorometer. Some embodiments of the present invention, have multiple light sources built into the hardware allowing user to select the desired light source.

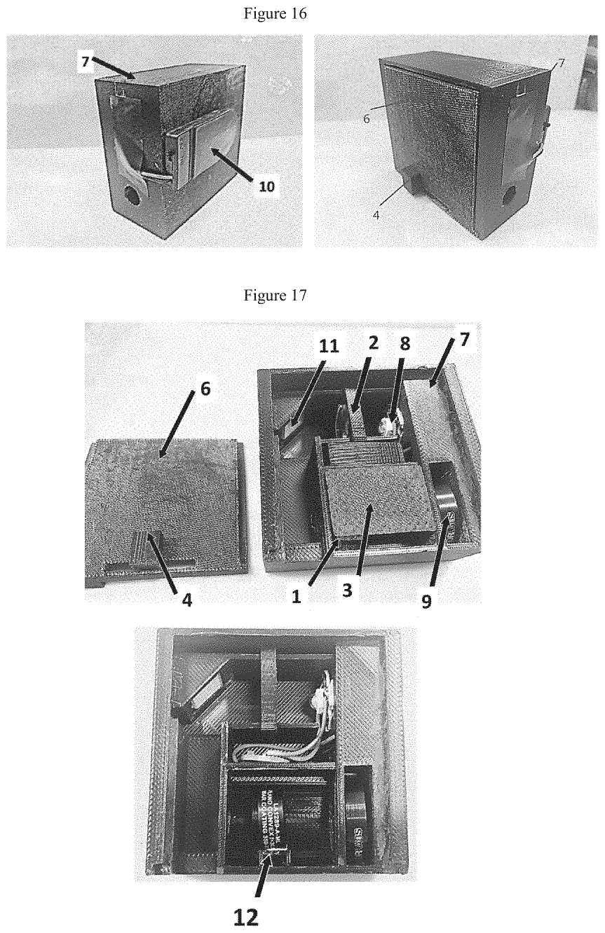

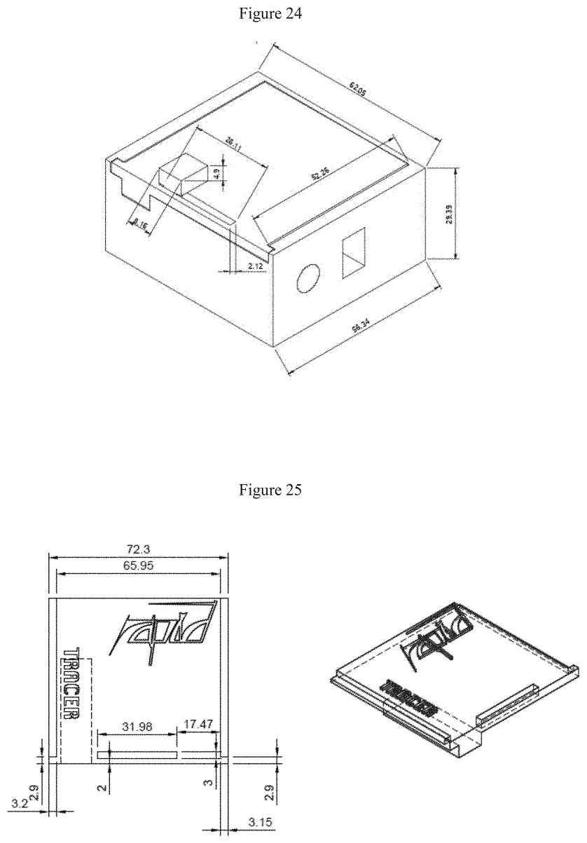

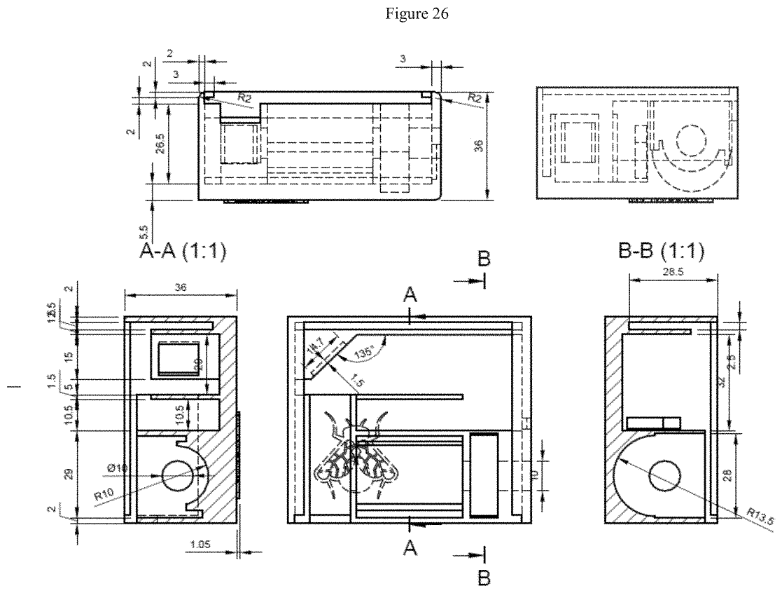

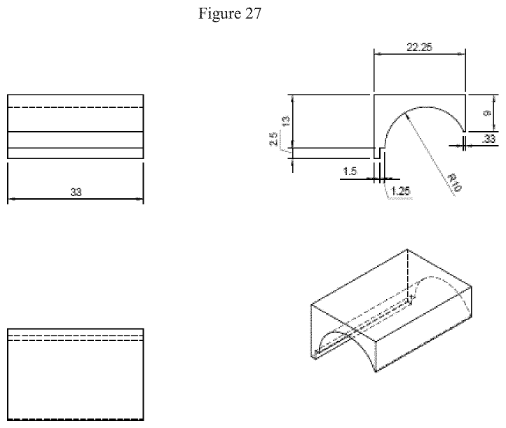

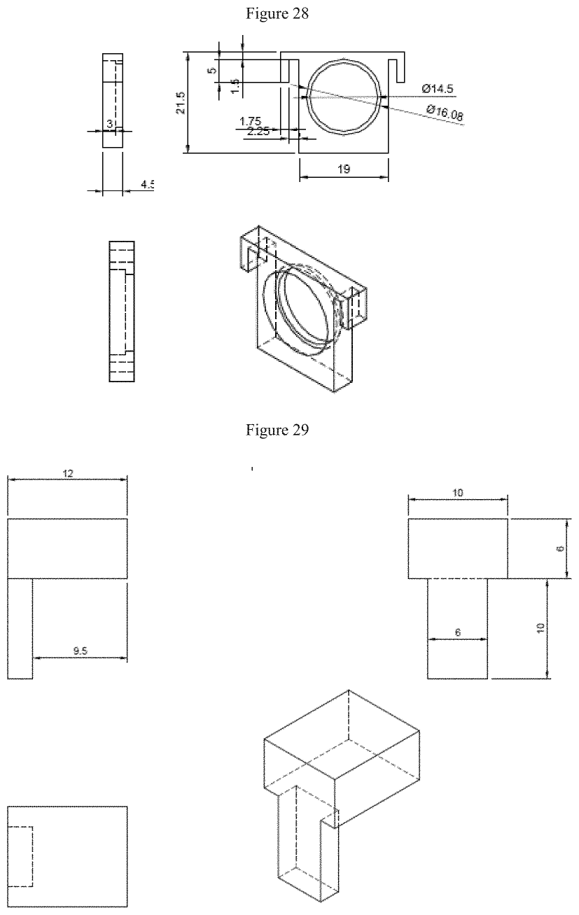

In some embodiments, the present invention provides a fluorometer. The various embodiments of the fluorometer are also referred to herein as Tracer. In some embodiments, Tracer is a battery-operated mobile-phone-based fluorometer which can not only record the intensity, but also the distribution and color pattern of the fluorescent signal. In some embodiments, Tracer is made up of a black housing made of polylactic acid (PLA), light emission system and optical system, as shown in FIG. 16. In one embodiment, the Tracer and its various parts are depicted in FIGS. 16-31.

The various embodiments of Tracer are designed to measure fluorescent signal given out by the RNA aptamer probes, but are also capable of detecting light signals given by other light-emitting moieties. In some embodiments, the power of Tracer is provided by replaceable battery cell.

In some embodiments, the housing is a black shell made of polylactic acid (PLA) and designed to optimize the measuring environment so that the light outside Tracer does not influence the measuring results. In some embodiments, the light-emitting diode emits visible blue light with 450 nm central wavelength, and is adjusted to parallel through a plane mirror and a convex lens. In some embodiments, the power supply system produces stable 700 mA current so that the light-emitting diode can work with a power of 5 Watt. In some embodiments, the battery box is placed on the back of Tracer so that users can replace the battery by themselves. FIGS. 17 and 18 depicts the internal structure of one embodiment of Tracer. In some embodiments, a different current strength and different wattage may be used. Analysis of the results can be adjusted based on these parameters.

In some embodiments, when Tracer is switched on, it emits excitation light with a central wavelength of 450 nm. This central wavelength corresponds to the peak value of the absorption spectrum for DFHBI. The central wavelength used may be selected based on the properties of a particular fluorophore. Fluorescent signals can be collected by mobile phone camera. In other embodiments, excitation light with different central wavelengths may be used. The choice of the excitation light wavelength depends on which fluorophen is used (see Bouhedda, 2018).

Some embodiments of Tracer have a shell made of black PLA This prevents the light outside from penetrating the shell so that the measuring results will not be affected by the environment. In other embodiments, the shell may be made of other materials and be of different colors. Various materials that prevent the light from penetrating the shell may be used.

In some embodiments, users can adjust the movable convex lens to help the mobile phone camera to focus. This lens may be referred to herein as focusing lens.

In some embodiments, the battery box is placed on the back of Tracer so that users can replace the battery by themselves. With an internal current regulator, the working power of LED remains stable at 5 Watt regardless of the battery voltage.

### Housing

In some embodiments, the present device comprises a housing for holding various components of the device. In some embodiments, the housing is a shell is made of black PLA. The Black PLA shell is a black box that holds all other components of tracer inside. The refractive index of PLA is as low as 3%, which can protect the diagnostics results from the influence of the outside environment. Moreover, with a melting point of around 160° C., PLA provides great heat stability. Also, PLA is an environmentally-friendly material as it is biodegradable.

In other embodiments, the shell may be made of other materials and be of different colors. A suitable material for the shell may be chosen based on such considerations as the materials' refractive index, melting point, light adsorption, weight, strength, durability and costs. If the refractive index is too high, it may be difficult to make the excitation light parallel. A reflective coating may be applied to the shells made of various materials to reduce or eliminate interference from the outside light.

TABLE 5
Characteristics of a shell representing
one embodiment of the present invention.

| | |
|---|---|
| Dimension | 62.05 mm × 56.34 mm × 29.39 mm |
| Thickness | 2.9 mm-3.1 mm |
| Weight | 72 g |
| Material | PLA |
| Color | Black |

### Light Emission System

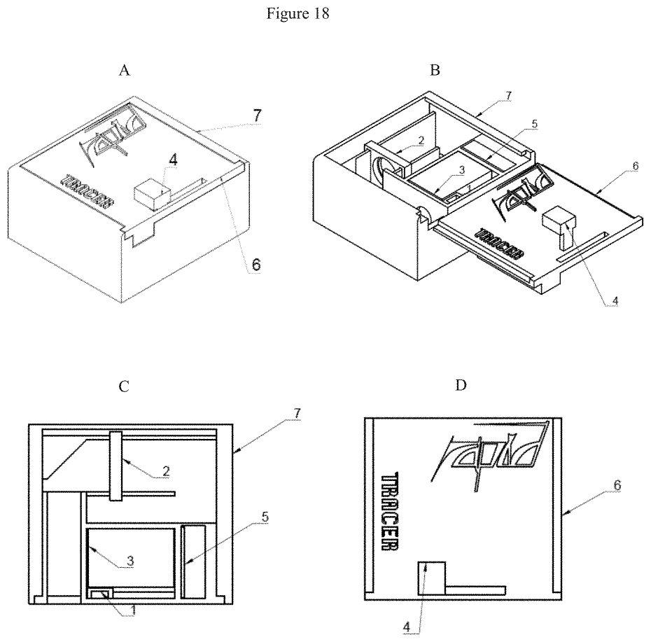

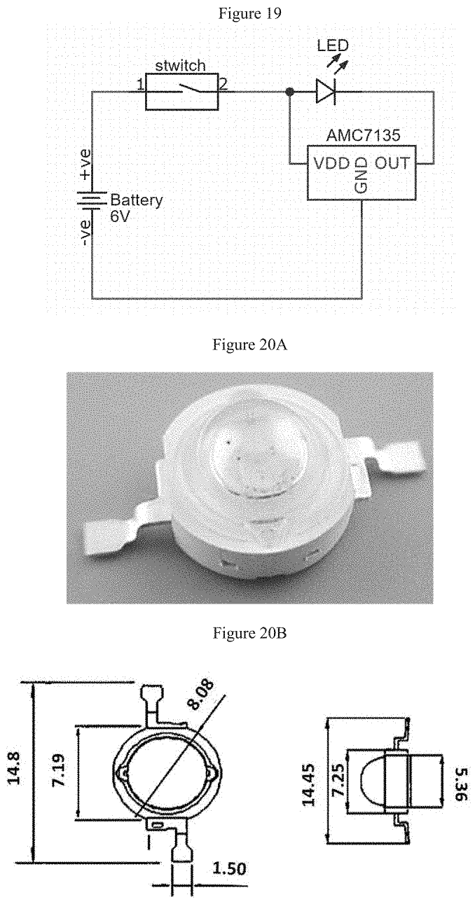

In one embodiment, the present device comprises a light emission system for generating light signals. In some embodiments, the light emission system provides Tracer with stable blue light with central wavelength of 450 nm. This central wavelength may be selected when DFHBI is used as a fluorophore as it corresponds to the peak value of the absorption spectrum for DFHBI. It consists of three parts: LED, LED current regulator and a power supply. FIG. 19 shows an electrical circuit design for a light emission system of a fluorometer device representing one embodiment of the present device.

### Light-Emitting Diode

FIGS. 20A and 20B show some embodiments of light-emitting diode that can be used in the present device. A different current strength and working voltage and other characteristics may be selected with the result analysis adjusted accordingly. A different waveband and the central wavelength may also be selected. The choice of waveband and wavelength may be optimized based on the particular fluorophore used. Waveband of 450-455 nm and the central wavelength of 450 nm work well when DFHBI is used as a fluorophore.

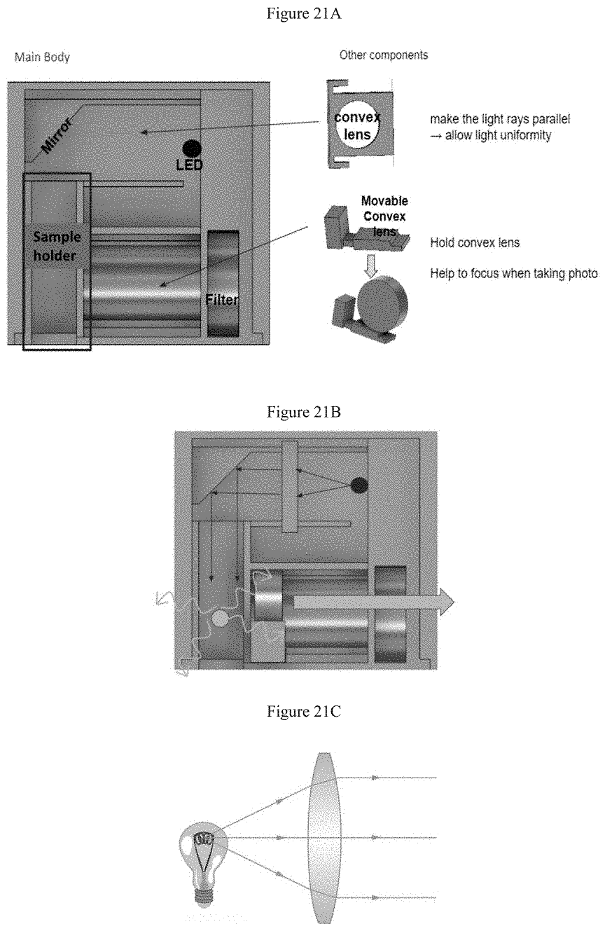

In some embodiment, as illustrated in FIGS. 21A and 21B, the light-emitting diode is located above the sample and the path of the emitted light. The direction of the excitation light is changed by 90 degrees with a mirror. This design ensures that the excitation light is not captured by the camera that captures the emission light (such as a mobile phone camera). Other design that eliminate or reduce the chance of the camera meant to capture the emission light also capturing excitation light may be used in various embodiments.

TABLE 6
Characteristics of a light emitting diode representing
one embodiment of the present invention.

| Dimension | See Graph | |
|---|---|---|
| Waveband | 450 nm-455 nm | |
| Light quantity | 50-60 | LM |
| Working voltage | 7-8 | V |
| Working current | 700 | mA |
| Maxima Energy Consumption | 5 | W |

### Heat Sink

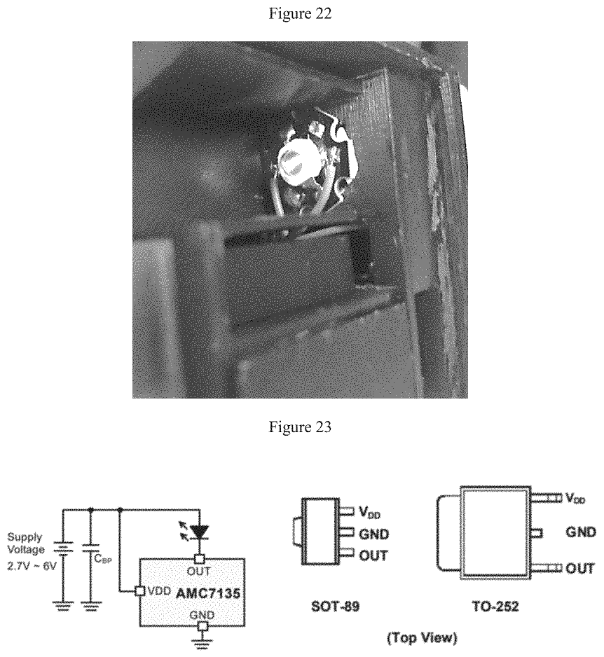

High power LED generates great amount of heat. Thus, in some embodiments, a heat sink such as a star heat sink is used to prevent overheating. In some embodiments, a start heat sink is used. FIG. 22 shows one embodiment of the heat sink that can be used in the present device. Other suitable configurations of heat sinks may be used.

### LED Current Regulator

LED current regulator can be included in some embodiments of the present device so that its performance will not be affected by the voltage of the battery. In some embodiments, two LED current regulators (AMC7135, from ADDtek, Taiwan) are used in parallel to regulate the current to 700 mA. FIG. 23 depicts one embodiment of the LED current regulator that can be used in the present device.

TABLE 7
Recommended operating Conditions and DC Electrical Characteristics.
RECOMMENDED OPERATING CONDITIONS

| Parameter | Symbol | Min | Typ | Max | Unit |
|---|---|---|---|---|---|
| Supply Voltage | V_{DD} | 2.7 | | 6 | V |
| Output Sink Current | I_{OUT} | | | 400 | mA |
| Operating Free-air | T_{A} | −40 | | +85 | ° C. |
| Temperature Range | | | | | |

DC ELECTRICAL CHARACTERISTICS
V_{DD} = 3.7 V, T_{A} = 25° C., No Load, (Unless otherwise noted)

| Parameter | Symbol | Condition | Min | Typ | Max | Unit | Apply Pin |
|---|---|---|---|---|---|---|---|
| Output Sink | I_{SINK} | V_{OUT} = 0.2 V | 340 | 360 | 380 | mA | OUT |
| Current | | V_{OUT} = 0.2 V, Rank A | 300 | 320 | 340 | mA | |
| Load Regulation | | V_{OUT} = 0.2 V to 3 V | | | 3 | mA/V | |
| Line Regulation | | V_{DD} = 3 V to 6 V, | | | 3 | mA/V | |
| | | V_{OUT} = 0.2 V | | | | | |
| Output Dropout | V_{OUTL} | | | 120 | | mV | |
| Voltage | | | | | | | |
| Supply Current | I_{DD} | | | 200 | | μA | VDD |
| Consumption | | | | | | | |

### Optical System

In one embodiment, the present device comprises an optical system for manipulating light signals. FIGS. 21A, 21B and 21C schematically depict one embodiment of an optical system and its components that can be used in the present device.

In some embodiments, the light emitted by light-emitting diode is converted to parallel by a convex lens and is reflected by the plane mirror. The plane mirror changes the direction of the light by 90 degrees and directs it toward the sample. The light excites the fluorophore and makes it emit fluorescent light (emission light). This fluorescent light is filtered by a bandpass filter so that the signal captured by the mobile phone camera is not affected by the excitation light. The light path of the excitation light and the emission light is designed to be perpendicular in order to minimize interference. In some embodiments, the convex lens is moveable to assist in camera focusing.

### Convex Lenses

In some embodiments, the divergent blue light emitted by the light-emitting diode is converted to parallel by the convex lens (FIG. 21C). In some embodiments, a convex lens has the following dimensions: length is 15.6 mm, center thickness 3.95 mm, edge thickness 2.5 mm, focal length 20 mm. This lens may be referred to herein as conversion lens.

In some embodiments, a moveable convex lens may be used. In some embodiments, the moveable convex lens is LA1289-A-ML convex lens with the following dimensions: diameter 0.5 inches. ARC 350-700 nm, weight 0.05 lbs. This lens may be referred to herein as focusing lens.

Lenses of other dimensions may be used on different embodiments. Smaller size lenses allow to minimize the overall dimensions of the device.

### Bandpass Filter

In some embodiments, the bandpass filter is used to filter out the excitation light so that it does not cause interference with the emission light. In some embodiments, Thorlabs FB510-10 Bandpass filter is used having the following dimensions: diameter 0.5 inches.

TABLE 8
Dimensions of a bandpass filter used in some
embodiments of the present invention.

| Diameter | Ø½ | inch |
|---|---|---|
| Auto CAD Dimension | See Graph | |
| CWL | 510 ± 2 | nm |
| FWHM | 10 ± 2 | nm |
| Weight | 0.05 | lbs |

### Operation

Tracers representing some embodiments of the present invention are operated as follows. A battery is installed. Sample is put into a sample holder of Tracer and the lid is closed. To give a satisfactory performance, lid of Tracer should not be opened when the Tracer is on. The Tracer is then switched on. Mobile phone camera is aimed at the signal collection port. Signal collection port is an opening in the shell of the device through which fluorescence may be observed and an image of the sample may be taken. The moveable lens is adjusted manually until a clear image is displayed on the mobile phone screen. The pictures are then taken with the mobile phone camera. The Tracer may then be switched off and the sample taken out of the Tracer.

### Performance Evaluation

Example 6 describes the components of an embodiment of Tracer and its estimated production cost.

Example 7 describes some procedures that were used to evaluate the performance of Tracer. The evaluation comprises two major parts: accuracy and precision.

### Operating and Image Processing System

In one embodiment, the present invention provides a system for operating the present device and processing images obtained by the present device. In some embodiments, the system has modules performing various functions, such as, calibration, image processing and machine learning.

At the time of this invention, a software or system that is capable of processing a large number of florescent images and equipped with machine learning for producing more accurate results was lacking.

### Calibration

In some embodiments, the present system comprises a module for calibration so that the present device is compatible with mobile phones of different configures (see Reference 13, for example).

### Image Processing

In some embodiments, the present system comprises a module for image processing.

Light-up aptamers provide a rapid, cheap and convenient way for on-site virus detection, which also brings possibility of self-detection method for the general public. To facilitate detection of virus by untrained public, a software representing one embodiment of the present invention may be used with mobile phone camera to detect fluorescent signal given off by light up aptamers. Though the software is currently used to detect fluorescent light, it can potentially be used to detect any color and light signal.

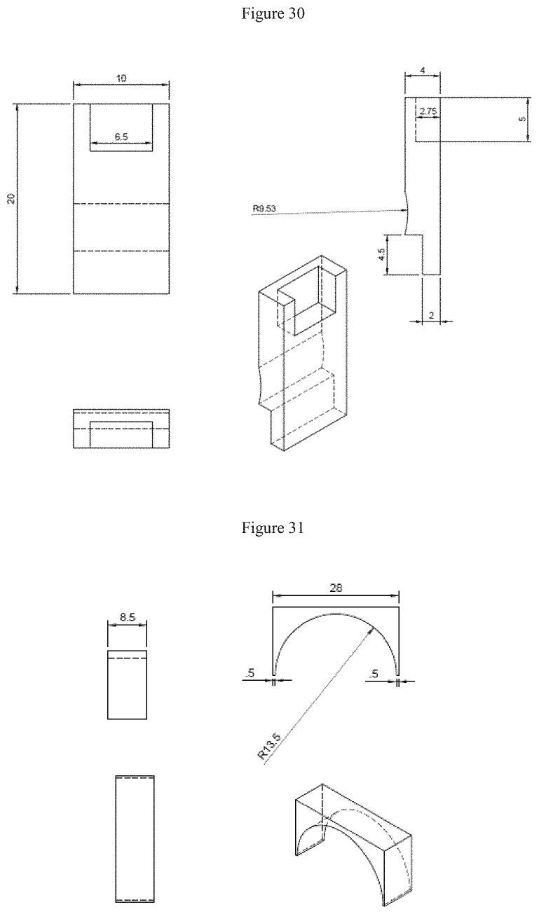

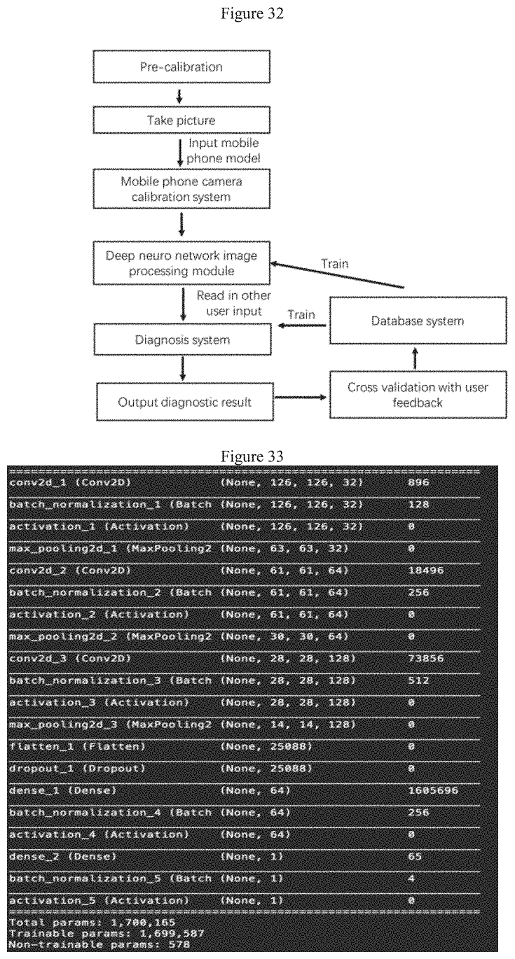

In some embodiments, the software includes five main parts: pre-calibration, mobile phone camera calibration module, deep neural network image processing module, diagnosis system and database system. FIG. 32 is a flowchart showing the functions of the image processing software in some embodiments of the present invention.

The software reads in the image uploaded by a user along with the mobile phone model number. Then the input image is calibrated based on the model number of the phone used to produce the image. The image is processed by the deep neural network image processing module. The diagnostic system outputs the diagnostic result based on the result of image processing and other information input by the user. If the result is positive, the user is suggested to see the doctor. After that, feedback is collected and used for training the image processing module and the diagnostic system.

In some embodiments, a convolution neural network is used. FIG. 33 depicts a design of the convolution neuro network of one embodiment of the present invention.

The main steps of the image analysis of some embodiments of the present invention are described below.

### Pre-Calibration

Pre-calibration eliminates possible background noise when no sample is inside the device. The user is asked to take three pictures using their own mobile phone without turning on the excitation light or putting in the sample. Before the image of the sample is processed, the average of the three images the user takes is subtracted to remove background noise.

### Mobile Phone Camera Calibration System

Users may use different mobile phones and color, brightness and other characteristics of the pictures may vary among different mobile phone camera. Moreover, mobile phone cameras can also introduce distortion to the images. Thus, a mobile phone camera calibration system is implemented to make sure that differences between different phone models do not influence the final diagnostic results. Since calibration takes a long time, calibration results of several popular mobile phone models are included in the database (e.g., iPhone). The calibration system is implemented based on OpenCV. Tw phantoms may be used for the calibration process.

### Distortion Calibration

The images taken by mobile phone camera can be distorted. In calibration of distortion, both tangential and radical distortion are considered (implementation details are described in reference 16). A 5×5 black and white phantom is used to calibrate the distortion of the camera in some embodiments of the present invention (see FIG. 34A).

### Color Correction

Same color may be different reproduced differently by different cameras. Therefore, a color correction module is implemented. A 24-color phantom for color correction is used in some embodiments of the present invention (see FIG. 34B). To perform calibration, the software locates different color regions; takes the average value of the pixels in the region as a representation of the RGB value of that square; then adjusts the gain of RGB value based on the underlying true RGB value of the color patch and the RGB values in the photos.

### Deep Neural Network Image Processing Module

This module uses a convolutional neural network to classify the input images into positive and negative classes. The input images will be convoluted by some convolution layers and pooled by pooling layers. At the end, the images will be classified by fully connected layers. The accuracy of the test data is at 83.3%. This module may be performed on local computers and trained on datasets locally obtained. It may also be performed on remote servers and/or utilizing cloud computing The module may be trained on locally generated datasets or on datasets generated at various locations and by various users and pooled together.

In another embodiment, the deep neural network image processing module employs a 3-layers convolutional neural network to classify the input images taken with the previously-described hardware into positive and negative classes. The input images will be convoluted by 3 convolution layers and be pooled by pooling layers. At the end, the images will be classified by fully connected layers. Each input image will be firstly resized with 128 in width and 128 in height before being fed into the neural network. The filters of the first convolution layer is 32 which means the output channel of this layer is 32 and the kernel size is 3*3 with stride 1. Following a batch normalization function, a relu function is employed as the non-linear activation function of the first layer. The following tow convolution layers are similar weight the first one except the filter size. The filter of the second convolution layer is 64 and that of the third convolution layer is 128. A 2*2 max pooling layer is attached after each convolution layer. Then, two fully connected layers are employed. The output dimension of the first fully connected layer is 64 and that of the last layer is one to indicate whether the image is positive or negative. The first fully connected layer employs a relu function as the activator while the second one employs sigmoid. Since the outcome is binary, a binary cross entropy loss function is applied.

### Diagnostic System

The symptoms and basic information of the user is also crucial to the diagnosis of influenza. To make the model available to users, a diagnostic system may be implemented on the website in some embodiments of the present invention. The diagnostic system may combine information collected from the user and the image processing result.

Users need to upload their images and the image will be classified by the neural network module. The result will return to users immediately. The online system contains only trained neural network model and is only used to test the user's images. The model is trained on local computers and the weights of the model will be updated routinely.

Information collected from the user may include: age; gender; geographic location; symptoms, such as body temperature, runny nose, sore throat, cough, muscle ache and other symptoms; when the symptoms start to occur; vaccination status. The types of information collected nay be adjusted based on the pathogens that are being detected using the present invention and/or disease that is being diagnosed.

### Database

The database of some embodiments of the present invention includes images with positive/negative annotation based on the experimental data, all the raw input data from the user and the user's feedback after he/she sees a doctor. The database is used to train the image processing and diagnostic system. Because geographical location data of the user may also be collected, the data may be used for disease control.

Other models may be designed to reduce the misclassification rate. The models may be trained with real clinical data. A self-calibration module may be included in some embodiments. If a user's phone model is not included in the database, the user can use the phantom inside the kit to calibrate the camera. After the user uses the phantom to calibrate the image, the calibration result may be included in the database.

Integrated System for Self-Detection of Influenza without Clinical or Laboratory Equipment

In one embodiment, the present invention provides a system adapted for self-detection of influenza virus by individual subjects without the need of any laboratory apparatuses or skills.

In one embodiment, the present invention provides an integrated system for a subject to conduct a detection of influenza virus outside of laboratory setting. In some embodiments, the present integrated system comprises one or more of: a module for collecting a sample of nasal fluid from a subject, a module for treating the collected sample with a detecting reagent comprising one or more fluorogen-bearing and influenza-specific probes, a light-shielded module for taking one or more images recording light emitted from the treated sample, and a module for processing the images and outputting results indicating the presence or absence of particular type or subtype of influenza virus. In some embodiments, the present integrated system is linked with a mobile phone of the user and is configured to enable the user to take images of their samples using their mobile phones and upload the images to the present integrated system for image-processing and analysis, and to receive results from the integrated system via the mobile phone.

In some embodiments, the present invention provides a method for self-detection of influenza virus by individual subjects without the need for any laboratory equipment or skills. In some embodiments, the method comprises:
i) Providing a strip containing one or more RNA aptamer probes provided by the present invention.
ii) Obtaining a sample from the subject and placing the sample in a tube.
iii) Adding the strip with RNA aptamer probes to the sample.
iv) Adding fluorogen to the sample and the aptamer probes.
v) Incubating the tube holding the strip and sample in hot water of 95-90° C. for about 5 minutes.
vi) Removing the strip from the sample.
vii) Placing the strip in a light-shielded environment to dry the strip.
viii) Placing the dried strip in Tracer.
ix) Operating Tracer as described herein.
x) Taking an image using a mobile phone.
xi) Uploading the image to a system for processing and analyzing the image.
xii) Retrieving the results generated by the system using the mobile phone.

Alternatively, the reaction can be done in a tube/cuvette instead of on a paper strip. The step may be:
i) Providing a tube containing in vitro transcription reaction mix for in situ synthesis of one or more RNA aptamer probes provided by the present invention and reconstituting it with water.
ii) Obtaining a sample from the subject and adding the sample to the tube.
iii) Incubating the mix at 37° C. for 1 hour.
iv) Incubating the tube in hot water of 95-90° C. for about 5 minutes.
v) Placing the tube in Tracer.
vi) Operating Tracer as described herein.
vii) Taking an image using a mobile phone.
viii) Uploading the image to a system for processing and analyzing the image.
ix) Retrieving the results generated by the system using the mobile phone.

In various embodiments, RNA aptamer probes may be provided embedded on a strip, freeze-dried in a tube or in other suitable form. In vitro transcription reaction mix for RNA aptamer probes may be provided instead of the RNA aptamer probes themselves. In such a case, in vitro transcription step is performed to obtain aptamer probes. Fluorogen may be added to the mix containing the patient sample and the RNA aptamer probes before or after the incubation step.

In some embodiments, image processing software is trained with positive and negative controls, such that a used does not need to also measure positive and negative control samples. In other embodiment, positive and negative control samples may be provided. Negative control may contain the same components as the sample obtained from the subjects except and a composition mimicking nasal fluid (or other biological material that may be used a sample for testing). Positive control samples may contain labeled probes and a known amount of the RNA they bind to as well as a composition mimicking nasal fluid (or other biological material that may be used a sample for testing).

In some embodiments, image processing software is trained with positive and negative controls, such that a used does not need to also measure positive and negative control samples. In other embodiment, positive and negative control samples may be provided. Negative control may contain the same components as the sample obtained from the subjects except the subject sample itself. Positive control samples may contain labeled probes and a known amount of the RNA they bind to.

In some embodiments results are uploaded through a website or a mobile phone application and the analysis may be performed on a remote server. In other embodiments, the image analysis software may be installed on the phone or a user computer itself.

The present invention can be adapted for detecting signals other than fluorescent signals. For example, light sources of various kinds can be added to the device so that user can determine which light they would like to use for various types of detections such as color detection, fluorescent detection and emissive light detection.

The present invention is applicable to samples of various kinds containing the target sequence. The sample can be a biological sample collected from the subject directly, or a sample derived from a biological sample collected from the subject. In one embodiment where the present invention is used for detection of influenza virus, applicable samples can be nasal fluid, saliva, tears or any other biological samples that contain viral genetic material.

The present invention provides a nucleic acid probe for detecting a target nucleic acid sequence. In one embodiment, nucleic acid probe of this invention comprises: (a) a fluorogen binding region comprising an aptamer sequence forming a G-quadruplex structure; (b) a first targeting sequence which interacts with a first portion of the target nucleic acid sequence; and (c) a second targeting sequence which interacts with a second portion of the target nucleic acid sequence; wherein interaction between the first targeting sequence and the first portion of said target nucleic acid sequence and interaction between the second targeting sequence and the second portion of said target nucleic acid sequence triggers conformational change of said G-quadruplex structure, which is then able to interact with a fluorogen in a way that induces fluorescence.

In one embodiment, the target sequence is a sequence present in the genome of a pathogen.

In one embodiment, the pathogen is influenza virus.

In one embodiment, the first targeting sequence comprises at least 11 nucleotides.

In one embodiment, the second targeting sequence comprises at least 11 nucleotides.

In one embodiment, the G-quadruplex structure in a stabilized form has a high binding affinity for a fluorogen than the destabilized form.

In one embodiment, the G-quadruplex structure gains stability and interacts with a fluorogen in a way that induces fluorescence when the first targeting sequence interacts with the first portion of the target nucleic acid sequence, or when the second targeting sequence interacts with the second portion of the target nucleic acid sequence, or both.

In one embodiment, the fluorogen binding region comprises the sequence of SEQ ID NO: 2 and SEQ ID NO: 4.

In one embodiment, the first targeting sequence comprises a sequence selected from the group consisting of even numbered sequences selected from the group of SEQ ID NO: 88-141.

In one embodiment, the second targeting sequence comprises a sequence selected from the group consisting of odd numbered sequences selected from the group of SEQ ID NO: 88-141.

In one embodiment, the fluorogen is 3,5-difluoro-4-hydroxybenzylidene imidazolinone (DFHBI).

In one embodiment, the binding of said probe to the target nucleic acid sequence enables said probe to interact with a fluorogen in a way that a visible fluorescent signal.

The present invention also provides a method for detecting a target nucleic acid sequence in a sample. In one embodiment, the method comprises: (1) providing a biological sample containing nucleic acids from a subject; (2) adding a nucleic acid probe and a fluorogen to said sample, wherein the nucleic acid probe comprises: a fluorogen binding region comprising an aptamer sequence forming a G-quadruplex structure; a first targeting sequence which interacts with a first portion of the target nucleic acid sequence; and a second targeting sequence which interacts with a second portion of the target nucleic acid sequence; (3) measuring fluorescence in said sample using a device capable of measuring fluorescence, wherein fluorescence indicates the presence of said target nucleic acid sequence.

In one embodiment, the target nucleic acid sequence is a nucleic acid sequence from a pathogen, and the nucleic acid probe is capable of hybridizing with said nucleic acid sequence.

In one embodiment, the pathogen is influenza virus.

In one embodiment, the device in step (3) is Tracer.

In one embodiment, the biological sample from the subject is one or more of the following: nasal fluid, saliva and tear.

In one embodiment, the target nucleic acid sequence is a nucleic acid sequence from a specific subtype of influenza virus, and the nucleic acid probe is capable of hybridizing to said target nucleic acid sequence.

The present invention further provides an imaging device configured for taking fluorescent images from a fluorescence-emitting sample using a mobile communication device. In one embodiment, the imaging device comprises (a) a housing comprising a movable opening and a signal collection port; (b) a sample holder to hold the fluorescence-emitting sample; (c) a power source; (d) a light source comprising one or more light emitting diodes and a current regulator; and (e) an optical module comprising a converging element, a focusing element and a filtering element.

In one embodiment, said device further comprises a heat exchanger.

In one embodiment, the converging element is a converging lens and the focusing element is a focusing lens that can be manually adjusted by a user.

In one embodiment, the sample is in liquid form at the time of imaging or has been deposited on a solid medium at the time of imaging.

In one embodiment, the power source is a battery.

In one embodiment, the present invention provides a method for detection of a target nucleic acid by an individual using said device, comprising the steps of: (1) providing a nucleic acid probe and a fluorogen to the sample, wherein the nucleic acid probe comprises: a fluorogen binding region comprising an aptamer sequence capable of forming a G-quadruplex structure; a first targeting sequence which interacts with a first portion of the target nucleic acid sequence; and a second targeting sequence which interacts with a second portion of the target nucleic acid sequence; (2) providing a biological sample from a subject; (3) combining the nucleic acid probe and the biological sample to obtain a test sample; (4) measuring fluorescence in the test sample using a device capable of measuring fluorescence to obtain fluorescence data and (5) determining whether the target nucleic acid is present in the sample by analyzing the fluorescence data obtained in step (4).

The present invention also provides an integrated system for detection of a target nucleic acid by an individual. In one embodiment, the integrated system comprises one or more of: a module for collecting a sample from a subject, a module for treating the collected sample with a detecting reagent comprising one or more fluorogen-bearing and influenza-specific probes, a light-shielded module for taking one or more images recording light emitted from the treated sample, and a module for processing the images and outputting results indicating the presence or absence of particular type or subtype of influenza virus.

In one embodiment, the system is used in conjunction with a mobile communication device, wherein the system is configured to enable the individual to do one or more of the following using the mobile communication device: take images recording light emitted from the treated sample, upload the images to the integrated system and receive results from the integrated system.

The present invention further provides a method for designing a sequence of an RNA aptamer capable of binding to a target nucleic acid, wherein the RNA aptamer forms a G-quadruplex structure that is capable of binding to a fluorogen, the method comprising: (a) selecting a target nucleic acid sequence; (b) generating a plurality of candidate sequences of an oligonucleotide having a hybridizing sequence substantially complementary to the target nucleic acid sequence; (c) evaluating the secondary structure of the candidate sequences and determining the likelihood of giving a fluorescence in the absence of the target nucleic acid by the candidate sequences; (d) for one or more of the candidate sequences, determining the binding probability between the candidate sequence and nucleotides involved in the formation or stabilization of the G-quadruplex structure, thereby determining the likelihood of giving a fluorescence in the absence of the target nucleic acid by the candidate sequence; (e) for one or more of the candidate sequences, determining the minimal free energy of one or more of: (i) the heterodimer of aptamer-target nucleic acid, (ii) the homodimer of aptamer, (iii) the homodimer of target nucleic acid, and the frequency of the aforementioned heterodimer and homodimer, thereby determining the likelihood of giving a fluorescence upon binding to the target nucleic acid by the candidate sequence; and (f) designing the sequence of RNA aptamer according to the results obtained in steps (c), (d) and (e).

The present invention also provides a system for designing a sequence of a RNA aptamer capable of binding to a target nucleic acid, wherein the RNA aptamer forming a G-quadruplex structure that is capable of binding to a fluorogen, comprising: (a) a sequence processing component for retrieving and processing sequence information, wherein said sequence processing component is further operable for (i) receiving a target nucleic acid sequence and selecting a portion of the target nucleic acid; and (ii) generating a plurality of candidate sequences having a hybridizing sequence complementary to the selected sequence; (b) a storage component for storing a training data set and a test data set for parameters indicative of the performance of the candidate sequences in binding to the selected sequence; (c) a structure prediction component for predicting the secondary structure of the candidate sequences and determining the likelihood of giving a fluorescence in the absence of the selected sequence by the candidate sequences; (d) a processing component comprising a machine learning component, wherein said processing component is further operable for (i) receiving data from and delivering data to said storage component; (ii) determining the binding probability between the candidate sequences and nucleotides involved in the formation or stabilization of the G-quadruplex structure, thereby determining the likelihood of giving a fluorescence in the absence of the selected sequence by the candidate sequence; (iii) determining the minimal free energy of one or more of: (i) the heterodimer of aptamer-target nucleic acid, (ii) the homodimer of aptamer, (iii) the homodimer of target nucleic acid, and the frequency of the aforementioned heterodimer and homodimer, thereby determining the likelihood of giving a fluorescence upon binding to the selected sequence by the candidate sequences; (iv) processing the data in the training data set to obtain a plurality of training data points; (v) processing the data in the test data set to obtain a plurality of test data points; (vi) testing the machine learning component using said test data points to obtain a test output; (vii) processing the test output and determining whether the test output is an optimal solution; and (viii) directing the sequence processing component to select another portion of the target nucleic acid based on the results of (vii).

In one embodiment, the module for processing the images and outputting results indicating the presence or absence of a particular type or subtype of influenza virus comprises pre-calibration module, calibration module, image processing module and diagnostic module.

In one embodiment, this invention provides a method for designing a probe for detecting a target sequence of nuclei acid in presence of a fluorogen, comprising the steps of: (a) selecting a target sequence; (b) selecting an aptamer sequence for forming a secondary structure comprising a fluorogen docking site that is destabilized; (c) generating one or more detecting sequences substantially complementary to a region on said target sequence and adding said one or more detecting sequences to an end of said aptamer sequence to form a probe sequence; (d) determining binding probability between complementary pairs of nucleotides in said probe sequence responsible for stabilizing of said fluorogen docking site; (e) obtaining value of one or more non-structural features related to said probe sequence and said target sequence; (f) Obtaining a first value indicative of probability of forming a heterodimer of said probe sequence and said target sequence from the results of (e); (g) Obtaining a second value indicative of probability of autofluorescence of said probe sequence from the results of (d); and (h) Determining if said probe sequence is a suitable probe candidate based on said first and second values.

In one embodiment, said non-structural features comprises: (a) minimal free energy of said heterodimer; (b) minimal free energy of a homodimer of said probe sequence; (c) minimal free energy of a homodimer of said target sequence; (d) value of delta G for binding of said heterodimer; and (e) frequency of minimal free energy structure of said heterodimer.

In one embodiment, said first value is obtained from the following equation:

first value=A(minimal free energy of homodimer of said probe sequence)+B (minimal free energy of homodimer of said target sequence)+C (minimal free energy of heterodimer of said probe sequence and said target sequence)+D (value of delta G for binding of heterodimer of said probe sequence and said target sequence)+E (frequency of minimal free energy structure of said heterodimer)
where, A, B, C, D and E are coefficients obtained by multiple linear regression based on the following equation:

on/off ratio=contant+first value+error.

In one embodiment, said second value is sum of binding probabilities between complementary pairs of nucleotides in said probe sequence responsible for stabilizing of said fluorogen docking site, wherein binding probability of each of said complementary pairs of nucleotides has a specific coefficient obtained by multiple linear regression based on the following equation:

mean fluorescent count=constant+second value+error.

In one embodiment, said aptamer sequence comprises SEQ ID NO: 143.

In one embodiment, said aptamer sequence comprises SEQ ID NO: 1 and SEQ ID NO: 5 to form a P1 arm linked to said fluorogen docking site. In another embodiment, said complementary pairs of nucleotides of step (d) comprises the nucleotides 1 to 4 of SEQ ID NO: 1 being complementary to nucleotides 7 to 4 of SEQ ID NO: 5 respectively.

In one embodiment, said one or more detecting sequences comprises two detecting sequences, each linked to an end of said aptamer sequence.

In one embodiment, said one or more non-structural features of step (e) is identified by: (a) determining normalized mutual information scores for a plurality of non-structural features of said probe sequence to identify a shortlist of non-structural features; and (b) conducting principal component analysis on said shortlist of non-structural features to identify said one or more non-structural features of step (e).

In one embodiment, said target sequence is a region in the genome of a pathogen. In another embodiment, said pathogen is an RNA virus. In a further embodiment, said RNA virus is selected from the group consisting of influenza virus, SARS-CoV, Zika virus and hepatitis C virus.

In one embodiment, the method of this invention further comprises the step of experimentally validating said probe sequence of step (h) and fine tuning said first and second values of steps (f) and (g).

In one embodiment, this invention provides a probe designed based on the method of this invention.

In one embodiment, said probe sequence comprises: (a) an RNA selected from SEQ ID NOs: 143-170; or (b) an RNA obtained by DNA transcription of SEQ ID NOs: 6-32.

In one embodiment, said one or more detecting sequences comprise a sequence selected from the group of SEQ ID NOs: 88-141.

In one embodiment, said fluorogen is 3,5-difluoro-4-hydroxybenzylidene imidazolinone (DFHBI).

In one embodiment, this invention provides a probe for detecting a target sequence of nuclei acid in presence of a fluorogen, comprising: (a) an aptamer sequence comprising SEQ ID NO: 143; and (b) one or more detecting sequences comprising SEQ ID NOs: 88-141.

In one embodiment, said probe comprises: (a) an RNA obtained by DNA transcription of SEQ ID NOs: 6-32; or (b) an RNA selected from SEQ ID NOs: 144-170.

In one embodiment, said one or more detecting sequences are two detecting sequences, each linked to an end of said aptamer sequence and complimentary to a continuous region on said target sequence.

### Example 1

### In Vitro Transcription (Cell-Free Production of RNA Aptamer Probes and Target RNA Molecules)

Oligonucleotides (oligos) that can hybridize to each other and be extended in a PCR reaction were designed in order to overcome the limitations in length of conventional oligo synthesis. After PCR using Phusion polymerase, the amplified DNA products were separated by gel electrophoresis and purified.

The purified DNA products were then used as a template in NEB HiScribe T7 Quick High Yield RNA Synthesis Kit for in vitro transcription. After DNasel treatment, the reaction was directly purified using 1:1 phenol:chloroform extraction, and the RNA concentration was measured with NanoDrop 2000. Molar concentration of the RNA probes and target RNA molecules were calculated based on their molecular weights according to their length.

FIG. 5 shows a schematic diagram of the above procedures and constructs for in vitro transcription.

### Example 2

### In Vitro Aptamer Refolding Essay

After in vitro transcription, resulting RNA aptamers (1 μM) were mixed with its 22-bp RNA target molecules (1 μM), 2× aptamer folding buffer (20 mM Tris-HCl, 200 mM KCl, 10 mM MgCl₂) and 1.5 μL of fluorophore DFHBI (200 μM)(Kikuchi N, 2016). Controls were set up by mixing the above components without RNA target molecule (aptamer only) or without RNA aptamer and RNA target molecule (blank), while the positive control was set up by mixing the aptamer folding buffer, DFHBI and untruncated miniSpinach. The mixtures were incubated in a dry bath at 90° C. for 5 minutes to allow refolding of the RNAs, and then incubated at 37° C. for 45 minutes Fluorescent signals were observed under blue light box (ChemiDoc) and the fluorescent intensity was measured by CLARIOstar plate reader at 447/501 Ex/Em.

Assay was done in triplicate and student's t-test was used for statistical analysis. Probability values (p-values) of 0.005 or less are regarded as statistically significant. The results are shown in FIGS. 6A-6C.

### Example 3

### Specificity and Sensitivity Study of the Aptamer Probes

The aptamer refolding assay as described in Example 2 was used for specificity and sensitivity analysis.

For specificity, aptamer probe candidates (1 μM) were mixed with their target or non-target sequences (1 μM) and DFHBI, the resulting fluorescence was measured. Blank consisted of only buffer, DFHBI and nuclease free water was prepared. The data were analyzed using Two-Way ANOVA (FIG. 8).

For sensitivity, 2 μM aptamer (N2-694 and N9-545 aptamers) was mixed with its target at different concentrations (i.e., 1.5 μM, 1 μM, 0.5 μM, 0.2 μM, 0.1 μM and 0.05 μM) and DFHBI, and the resulting fluorescence was measured. Blank consisted of only buffer, DFHBI and nuclease free water. Detection limit of the aptamer probes was determined by fluorescent signals measured by CLARIOstar plate reader with Ex/Em 447/501 and visually by photos taken by ChemiDoc Imager under SYBR Green mode with Blue Trans Light Excitation (FIGS. 8A and 8B).

### Example 4

### Ion Dependency Study

Folding of the present RNA aptamer probes in the presence of sodium ion, potassium ion, calcium ion or magnesium ion in different concentrations were tested.

Modified from aptamer refolding assay described in Example 2, ions were added to the aptamer folding buffer in the form of salt solution. Table 9 lists the concentration of different ions in nasal fluid and in aptamer folding buffer. Table 10 lists the range of concentrations of each type of ion in the final reaction mixtures. FIGS. 9A-9D show the results.

TABLE 9
Concentration of different ions in nasal fluid and in aptamer
folding buffer (note that the aptamer folding buffer used composed
10 mM Tris-HCl, 100 mM KCl, 5 mM MgCl₂).

| | Sodium | Potassium | Calcium | Magnesium |
|---|---|---|---|---|
| | ion | ion | ion | ion |
| Concentration in nasal fluid | 138- | 31- | 1.00- | 0.47- |
| (mM) (Burke W., 2014) | 189 | 40 | 1.85 | 1.17 |
| Concentration in aptamer | 0 | 100 | 0 | 5 |
| folding buffer (mM) | | | | |
| Predicted concentration in | 138- | 131- | 1.00- | 5.47- |
| reaction mixture after | 189 | 140 | 1.85 | 6.17 |
| adding nasal fluid to | | | | |
| freeze dried | | | | |
| buffer (mM) | | | | |

TABLE 10
Range of concentrations of each type
of ion in the final reaction mixtures

| Sodium | Potassium | Calcium | Magnesium |
|---|---|---|---|
| ion (mM) | ion (mM) | ion (mM) | ion (mM) |
| 0 | 0 | 0 | 0 |
| 50 | 10 | 2 | 5 |
| 100 | 20 | 4 | 10 |
| 150 | 30 | 6 | 15 |
| 200 | 40 | 8 | 20 |
| 250 | 100 | 10 | 40 |
| / | 200 | 20 | / |

### Example 4

### Optimization of Aptamer Folding Condition

Bio-Rad Real-time PCT system was used to monitor the change in fluorescent signals and the time required for signal development.

Reaction mixtures in a 96-well plate were set up as follows: aptamer probe (1 μM), target RNA (1 μM), 30 μL of 2× folding buffer, 1.5 μL of DFHBI (200 μM) and nuclease-free water to make a final volume of 50 μL.

Aptamer control was prepared similarly by replacing the target RNA by nuclease-free water and untruncated miniSpinach positive control.

The thermocycler was set up as follows:
a. 95° C. for 5 minutes
b. 94° C. for 30 seconds, decrement temperature by 1° C. per cycle, total 90 cycles. Readings were taken continually.
c. Melting Curve: 4° C. to 95° C., increment 0.5° C. every 5 seconds. Readings were taken continually.

The reaction mixtures were first heated at 95° C. for 5 minutes, then allowed to be cooled slowly to 25° C. FIG. 10A shows the time and temperature for signal development of N9-694 and N2-545 probes (N=1).

For melting curve (dissociation) analysis, reaction mixtures which underwent refolding as described in the preceding paragraph were heated from 4° C. to 95° C. over the course of one hour. FIG. 10B recorded the melting curves of N9-694 and N2-545 probes (N=1).

### Example 5

### Production and Screening of RNA Aptamers in E. coli

RNA aptamer probes and their RNA targets were co-expressed using the Novagen Duet vector system in E. coli using standard procedures for co-expression of recombinant proteins in E. coli.

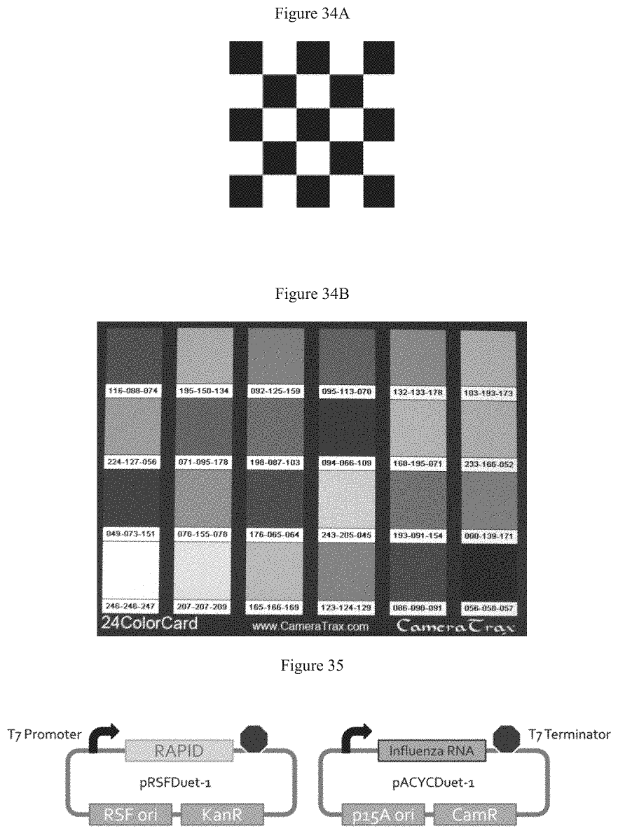

pRSFDuet-1 vector was modified to generate a T7 promoter-based aptamer expression system, and target genes were cloned into the multiple cloning site of pACYCDuet. FIG. 35 shows the resulting constructs where the left panel shows the construct with RNA aptamer probes (RAPID) while the right panel shows the construct with target RNA (Influenza RNA). Transcription and hence expression of the cloned construct can be triggered by adding IPTG.

After IPTG induction in co-transformed BL21 Star (DE3) (provided by http://2018.igem.org/Team:Hong_Kong-CUHK/Collaborations NUS Singapore-A team), the cells were collected and resuspended in a medium containing 200 μM DFHBI, fluorescence was measured by BMG CLARIOStar microplate reader (FIG. 15A). Total RNA was extracted from the same number of cells as was used in the whole cell assay and tested for its fluorescence level in the presence and absence of the target sequence. RNA probes produced using in vitro transcription were also tested. The results are shown in FIG. 15B.

MiniSpinach was used as a positive control.

### Example 6

### Configurations and Costs for Producing a Battery-Operated and Mobile-Phone Based Device for Measuring Fluorescent

This example describes the components of a device representing some embodiments of the presence invention and its estimated production cost.

The following components were used:
Battery: CR2032 (thin and small, relatively high voltage);
Battery Box;
5 W LED: wavelength 450 nm (blue light);
LED current regulator: AMC7135 (capable of maintaining constant light intensity);
Cuvette;
Bandpass Filter: CWL 500;
Mirror: 10 mm×10 mm;
Convex lens with the following characteristics;
   Focal length: 20 mm (conversion lens)
   Focal length: 30 mm (focusing lens)
PLA: 72 g
Cooling plate

Table 11 provides estimated costs of manufacture. The prices are shown as of September 2019 and assuming that 100 filter are purchased.

TABLE 11
Estimated costs of manufacturing a device representing
one embodiments of the present invention.

| | Price in HK$ × number |
|---|---|
| | of parts needed |
| Part | for one device |
| Battery: CR2032 | 1.2 × 2 |
| Battery Box | 0.5 × 1 |
| 5 W 450~455 nm LED | 7.5 × 1 |
| LED Voltage Regulator AMC7135 | 2.9 × 1 |
| Sample tubes | 0.01 × 1 |
| Bandpass Filter | 11.5 × 1 |
| Mirror | 0.6 × 1 |
| 20 mm - focal length convex lens | 2.7 × 1 |
| 30 mm - focal length convex lens | 2.7 × 1 |
| PLA 72 g | 11.3 |
| Cooling Plate | 0.3 × 1 |
| Total | 42.4 HKD ≈ 5.44 USD |

### Example 7

### Evaluation of Battery-Operated and Mobile-Phone Based Device for Measuring Fluorescence

This example describes the procedures for evaluating the performance of Tracer of example 6. The evaluation comprises two major parts: accuracy and precision.

### Accuracy

Plate reader routinely used in laboratories was used as a comparison device for Tracer. The mobile phone used to collect signal was iPhone 6S. E. coli cells expressing GFP were lysed and serial dilutions of green fluorescent protein (GFP) solution were prepared without further GFP purification and used as test samples. GFP solutions with relative concentrations of 0.00001 to 0.5 were prepared. The samples to be measured by Tracer were transferred to sample tubes, while samples to be measured by the microplate reader were transfer into microplates (tubes from Gene Company LTD, part #23140 were used in this experiment). Samples were put one-by-one, into Tracer for detection. Images were collected using the mobile phone. The obtained images were analyzed using matlab as follows: the detected light was first outlined on the image, the image was converted into greyscale and the average relative light intensity was calculated. For comparison, fluorescent signals of each sample were measured using the plate reader. Each sample was measured three times and the measurements averaged. Results are shown in Table 12.

TABLE 12
Results of accuracy study showing the determined fluorescence
intensity levels measured by the two methods (plate
reader and Tracer with mobile phone camera)

| Relative concentration | Plate reader | Tracer + matlab |
|---|---|---|
| 0.5 | 237158 | 930.5415 |
| 0.25 | 118747 | 716.7816 |
| 0.2 | 130147 | 342.6431 |
| 0.1 | 65465 | 264.254 |
| 0.05 | 33551 | 94.72892 |
| 0.025 | 26310 | 83.20531 |
| 0.02 | 12708 | 72.20319 |
| 0.01 | 10413 | 33.57129 |
| 0.005 | 6127 | 28.23181 |
| 0.001 | 11640 | 5.695414 |
| 0.001 | | 4.827621 |
| 0.0001 | 16487 | 3.45028 |
| 0.00001 | 15992 | 1.850767 |
| Water | 9685 | 2.306944 |

### Analysis of Accuracy Data

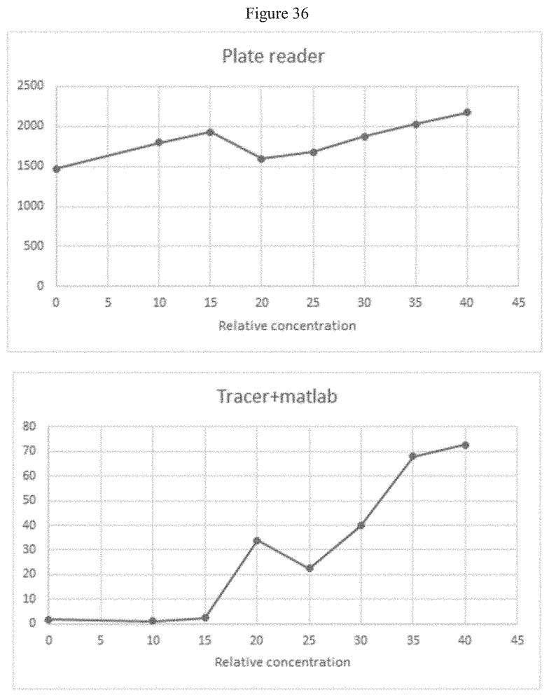

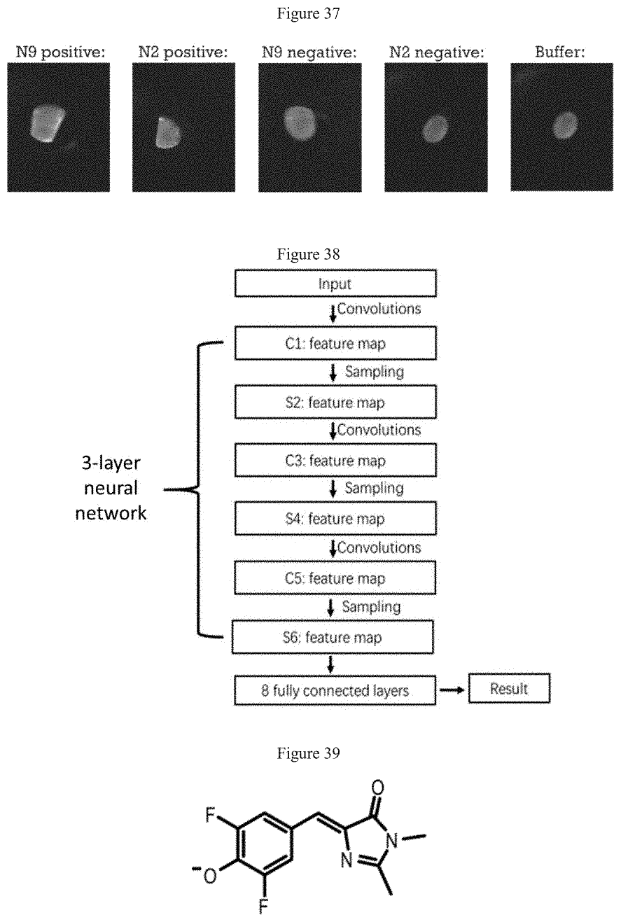

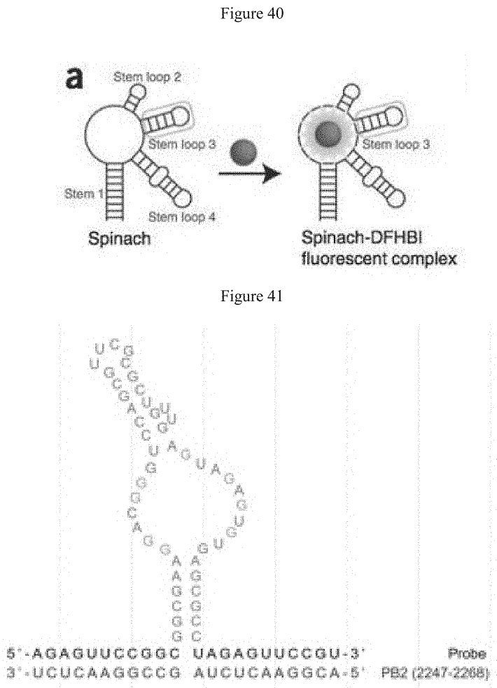

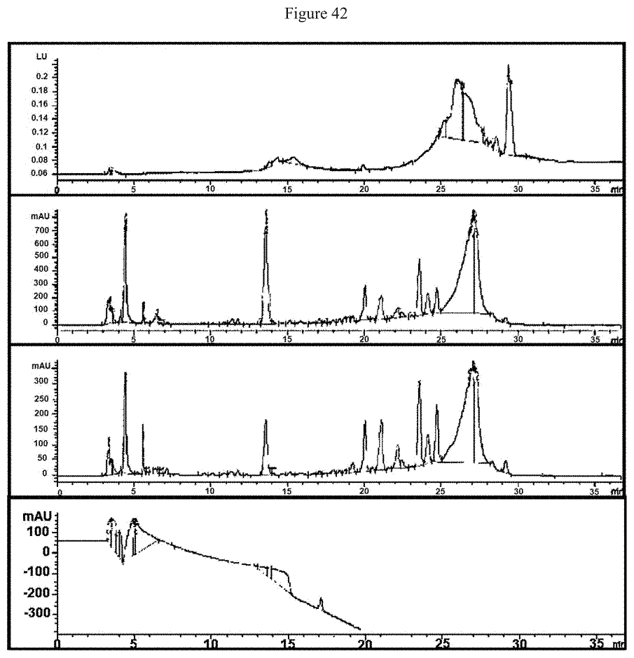

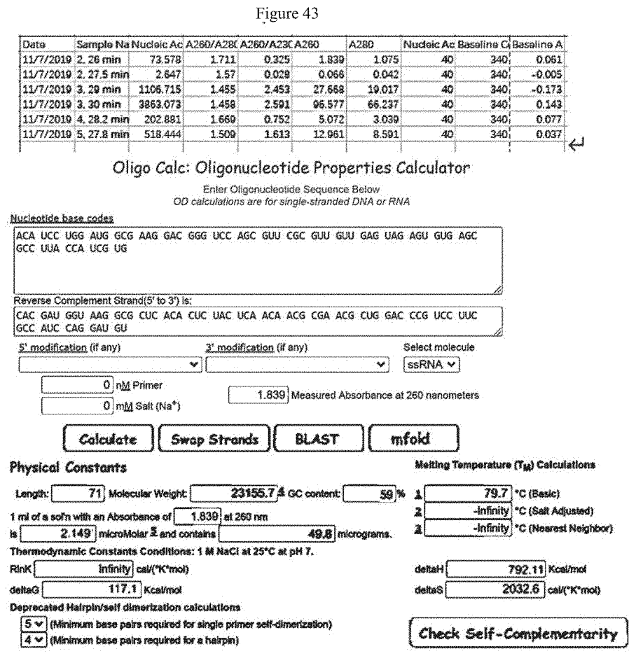

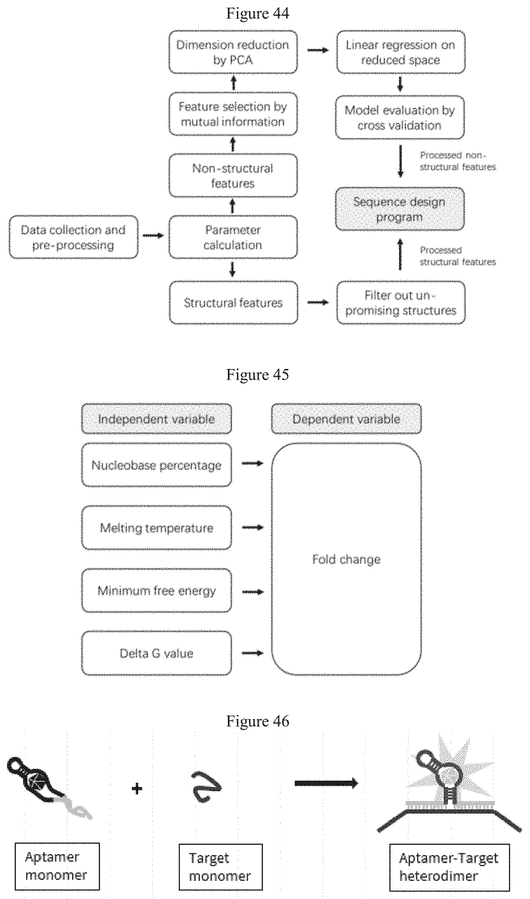

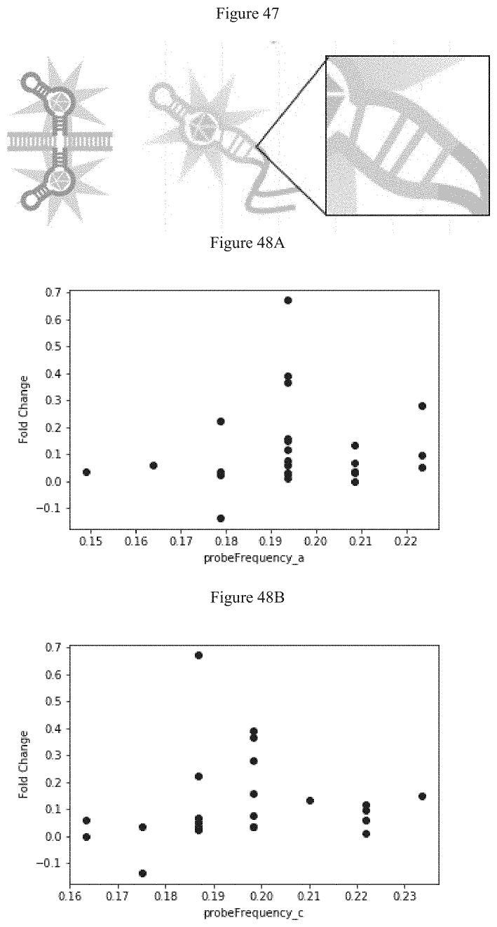

As can be seen from the graph in FIG. 36, the accuracy of Tracer is quite high compared to the results obtained from plate reader. However, at the low concentration region it becomes hard for tracer to distinguish between different concentrations. The same was observed in precision measurements. The reasons might be limitation of picture resolution of the mobile phone camera itself or the fact that some of the light is reflected by the surface of the tube. As a result, as can be seen from FIG. 37, most of the signal comes from the surface of the tube when the concentration is low. This also indicates that intensity may not be enough to distinguish positive and negative signals, and distribution is also an important parameter. This also accounts for the reason why machine learning may be a suitable tool to process and analyze the signal image.

### Precision

GFP solutions with concentration very close to each other were prepared. The range of fluorescent intensity level of the GFP solutions was made comparable with that of the present RNA aptamer probe. The measurements and analysis was performed as described above for the accuracy determination.

TABLE 13
Results of precision study showing the determined fluorescent intensity
levels measured by the two methods (plate reader and Tracer)

| Relative concentration | Plate reader | Tracer + Matlab |
|---|---|---|
| 40 | 2176 | 72.623 |
| 35 | 2032 | 68.00643 |
| 30 | 1877 | 39.92452 |
| 25 | 1678 | 22.42998 |
| 20 | 1596 | 33.91813 |
| 15 | 1931 | 2.365423 |
| 10 | 1797 | 1.086691 |
| 0 | 1473 | 1.717199 |

### Analysis of Precision Data

As can be seen from the overall trend of the graphs in FIG. 36, Tracer can detect the difference of fluorescent emitted from different samples with slight differences in their concentrations. But similar to the above analysis for accuracy, at low concentrations both plate reader and Tracer showed decreased precision.

### Example 8

### Method for Rational RNA Aptamer Design

In this example, a rational way of designing RNA aptamer is proposed. Based on observation of experimental data and literature review, 20 non-structural parameters and 3 structural parameters that can potentially affect the performance of Spinach aptamer are identified. Then feature selection based on mutual information and dimension reduction by principal component analysis is used to analyze the 20 non-structural parameters. Finally, multivariate linear regression is performed on the experimental data to generate a scoring function that can be used to predict the performance (fold change) of aptamers. Using the scoring function and other structural information, a program that can screen and select RNA aptamer designs is designed.

### Aptamer Design

The aptamers are designed to target a 22-bp subsequence of influenza virus. Spinach is a sequence of RNA that can bind with 3,5-difluoro-4-hydroxybenzylidene imidazolinone (DFHBI) and give out green florescent light.

According to its crystal structures, Spinach consists of two arms, P1 and P2, surrounding a docking site of DFHBI form by a G-quadruplex. After truncating the P1 arm of Spinach, a destabilized form of Spinach is obtained. Then a 11-bp arm is added to each side of the truncated sequence. Note that the two 11-bp sequences added are complimentary to the 22-bp subsequence being targeted. After modification, Spinach aptamer will only fold correctly and light up when hybridized to the target RNA (influenza RNA) (Ong, 2017). FIG. 3 shows how the design works.

### RNA Synthesis and Testing

### RNA Synthesis

In this example, 2 pairs of aptamer and target are generated:

TABLE 14
Sequence list of the 2 pairs of aptamer
and target generated

| Sequence | Chem | |
|---|---|---|
| name | property | Sequence |
| N9-545 | RNA | SEQ ID NO: 142 |
| aptamer | | acauccuggauggcgaaggacggguccagcgu |
| | | ucgcgcuguugaguagagugugagcgccuuac |
| | | caucgug |
| N9-545 | RNA | SEQ ID NO: 46 |
| target | | uguaggaccua |
| | | SEQ ID NO: 47 |
| | | aauuguagcac |
| H7-474 | RNA | SEQ ID NO: 163 |
| aptamer | | ugacaggagccggcgaaggacggguccagcgu |
| | | ucgcgcuguugaguagagugugagcgccauuu |
| | | cauuucu |
| H7-474 | RNA | SEQ ID NO: 72 |
| target | | acuguccucgg |
| | | SEQ ID NO: 73 |
| | | uaaaguaaaga |
| Mini | RNA | SEQ ID NO: 171 |
| spinach | | gggagaaggacggguccaacguucgcgcuguu |
| | | gaguagagugugagcuccc |

Oligo synthesis is used for both RNA aptamers and target sequence. The products are purified by HPLC purification, and concentrations were calculated by nanodrop.

### Testing the Performance of Aptamer

1 uM aptamer, 1 uM target and 1.5 ul 200 uM DFHBI are mixed in buffer (20 mM Tris-HCl, 200 mM KCl, 10 mM MgCl2, PH=7.5), then refolded in 90 degree Celsius for 5 minutes, incubated in 37 degree Celsius for 45 minutes (2018 iGem team).

### Data Analysis and Modeling

Influenza virus RNA has a total length of around 14,000 nucleotides (Duesberg, 1968), while aptamer can only detect a short strand (usually 20-80 bp). Therefore, it is impossible to detect the whole RNA strand. A proposed solution is to target only 22-bp subsequence of influenza. Since the performance of aptamer change when the target subsequence is changed, how to choose the target subsequence rationally becomes an important question. This part will discuss how a model can be built and the performance of an aptamer design be predicted based on its sequence.

### Data Collection and Preprocessing

26 pairs of target and probe sequences are designed, synthesized, and tested for data analysis. Note that since the data size is very small, some special techniques such as dimension reduction and cross-validation will be introduced in later sections to avoid overfitting.

### Feature Selection and Dimension Reduction

### Potential Parameters

Through observation and literature review, some features that can potentially affect fold change are listed below. These features are divided into two main types, structural and non-structural. Non-structural features are calculated from the sequences of targets and probes. While structural feature refers to secondary structure predicted by calculating minimum free energy and binding probability. Non-structural features can be easily quantified. Therefore, regression is used to model them; Structural features are hard to quantify; thus, they are mainly used to reject unpromising designs.

Non-Structural Features:

The performance of Spinach aptamers is quantified by fold change. Fold change indicates how many times fluorescent level changes before and after target are added to Spinach aptamer solution.

Non-structural features are classified into 4 types: nucleobase percentage, melting temperature, minimum free energy, and delta G value. In this section, the relationship between quantified non-structural features and fold change will be found.

1. Nucleobase Percentage

Nucleobase percentages refer to the ratio of A, G, C, and U in target and probe. Since A-U forms a double hydrogen bond and C-G forms a triple bond. The ratio of C-G pairs may affect the thermal stability of the probe-target heterodimer.

2. Melting Temperature

The melting temperature is the temperature at which 50% of the base pairs have been broken. It gives information on when and how RNA strands hybridize and is therefore a potential parameter that can affect the binding of the probe and target. The analysis take into consideration both target MFE and probe MFE.

3. Minimum Free Energy

One RNA sequence can have different secondary structures under the same condition, and structure with minimum free energy (MFE) is the most thermodynamically stable one. The MFE of target, probe, target-probe heterodimer, target-target homodimer, and probe-probe homodimer are considered.

4. Delta G

Delta G value is closely related to the thermodynamic stability of the reaction product. Delta G for Heterodimer binding (P-T), Delta G for Homodimer binding (P-P), and Delta G for Homodimer binding (T-T) are all considered.

Structural Features:

There are two types of errors in aptamer design. Type one error, also known as a false negative, indicates that the probe-aptamer heterodimer does not fold correctly and cannot bind with DFHBI. Type two error refers to false positive, meaning that florescent is detected when there is no target presenting. Type two error is caused by the misfolding of one or multiple probe strings.

Secondary structures of candidate aptamer can be predicted by calculating the minimum free energy using the Vienna RNA python package (Hofacker, 2003). If the predicted secondary structure of a probe monomer or a probe-probe homodimer indicates the formation of the G-quadruplex docking site of DFHBI, the aptamer design is likely to have high false positive error (light-up without target). In the program, such aptamer designs will be discarded. Similarly, if the MFE structure of probe-target heterodimer does not form a binding structure, the design is predicted to have high false negative rate (does not give the signal when target is presenting) and will also be discarded.

TABLE 15
Errors indicated by structural features

| Type I error (false negative) | Type 2 error (false positive) |
|---|---|
| Wrong structure form | Light-up structure formed |
| by probe-target | without target |

### Visualization of Non-Structural Parameters

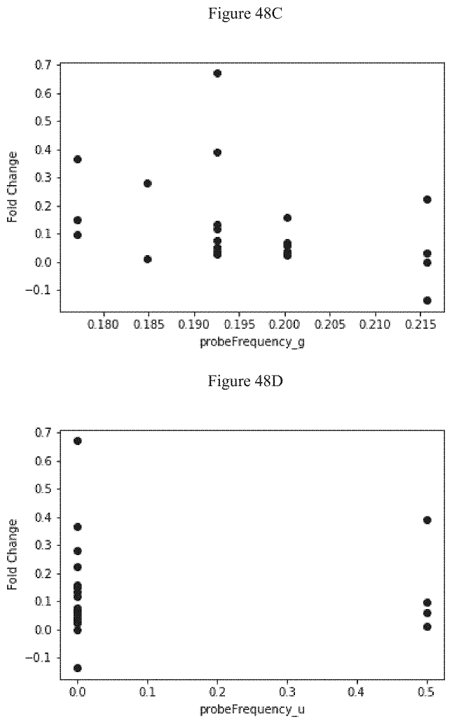

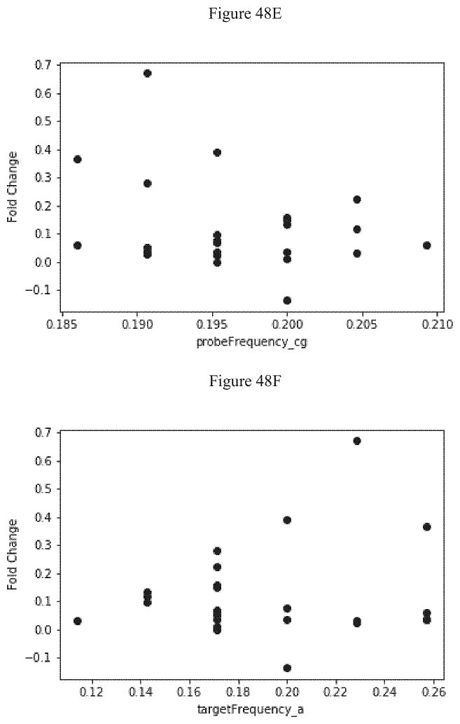

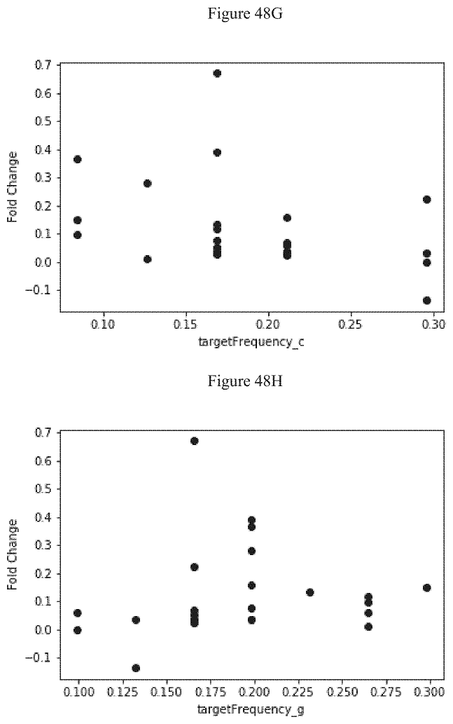

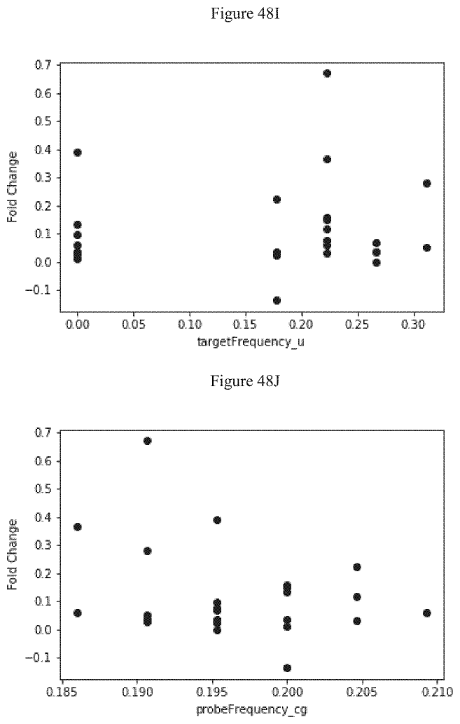

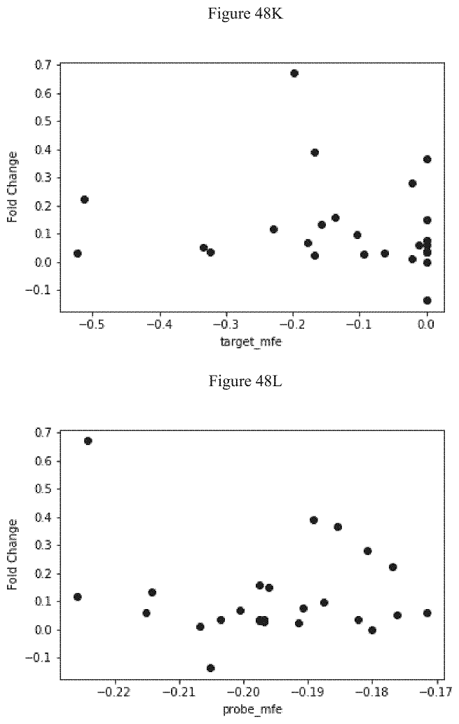

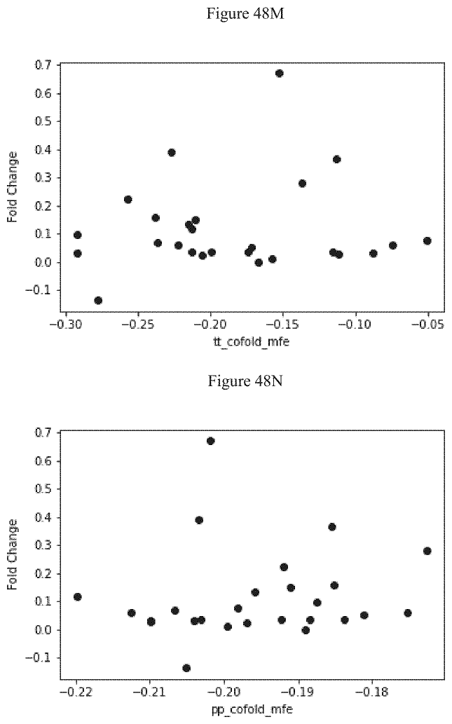

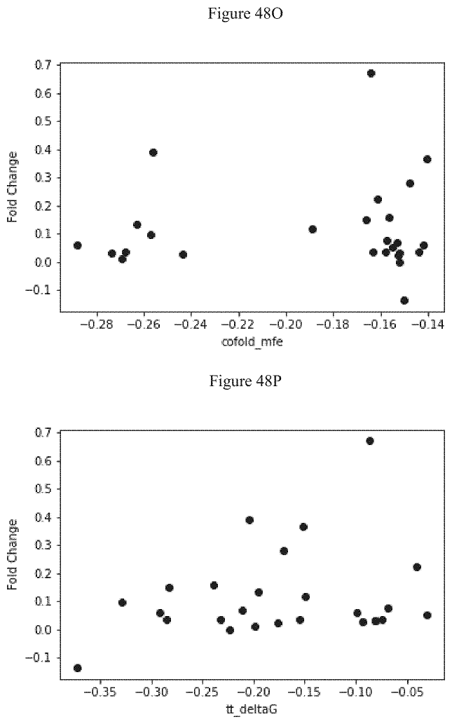

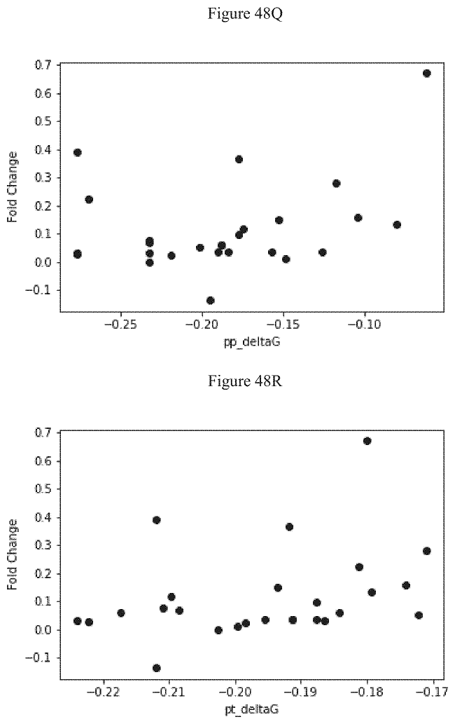

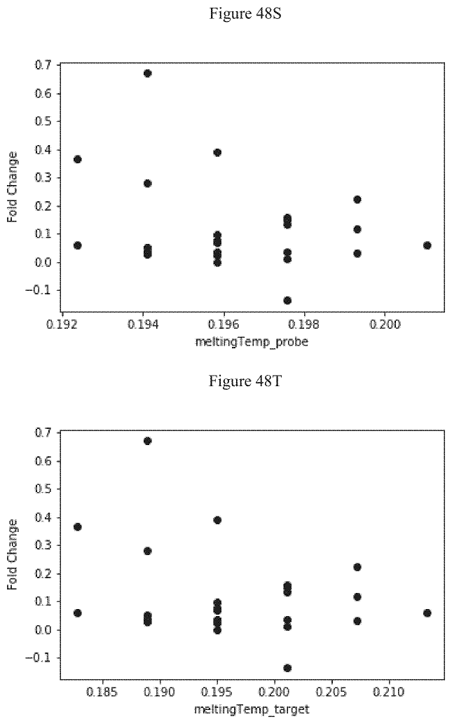

FIGS. 48A-48T show diagrams of fold change versus each non-structural parameter are plotted.

### Normalized Mutual Information (NMI) Feature Selection for Non-Structural Features

To see whether each parameter can indeed indicate the value of fold change, normalized mutual information is calculated for each non-structural feature. This step is mainly used to filter out less relevant features.

TABLE 16
Results of normalized mutual information feature selection

| | | | Normalized Mutual |
|---|---|---|---|
| | | | Information (NMI) |
| Category | | Parameter | with fold change |
| Nucleobases | Probe | A % (P) | 0.702 |
| percentage | | G % (P) | 0.668 |
| | | C % (P) | 0.712 |
| | | U % (P) | 0.678 |
| | | GC % (P) | 0.704 |
| | Target | A % (T) | 0.676 |
| | | G % (T) | 0.712 |
| | | C % (T) | 0.668 |
| | | U % (T) | 0.363 |
| | | GC % (T) | 0.704 |

| | | |
|---|---|---|
| Melting temperature | MT (T) | 0.856 |
| | MT (G) | 0.960 |
| Minimum free energy (MFE) | MFE (P) | 0.991 |
| | MFE (T) | 0.983 |
| | MFE (PP) | 0.991 |
| | MFE (TT) | 0.704 |
| | MFE (PT) | 0.704 |
| Delta G value | Delta G (PP) | 0.928 |
| | Delta G (TT) | 0.998 |
| | Delta G (PT) | 0.991 |

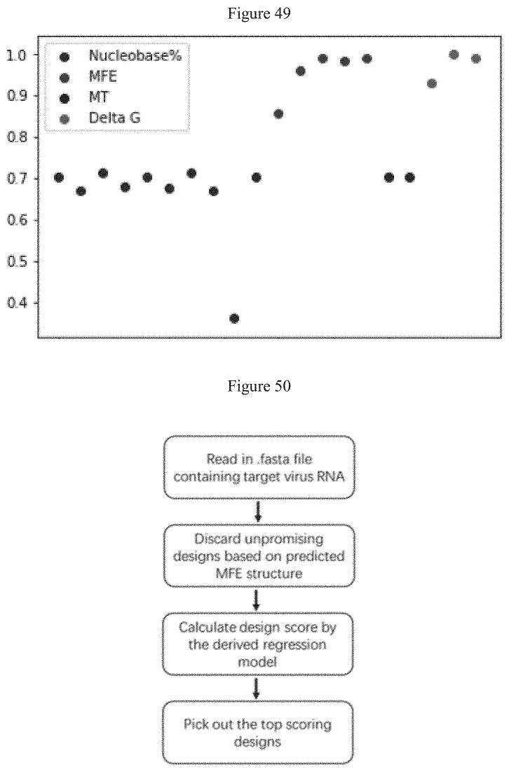

FIG. 49 visualizes the results of NMI for each category. As can be seen from the plot, thermodynamic features (MFE, delta G) seem to best indicate fold change. Notably, the U % of the target seems to have a rather low mutual information score (0.363). Thus, this feature is discarded and proceeded with the remaining 19 features.

### Dimension Reduction by Principal Component Analysis (PCA)

Principal component analysis is a common technique to reduce the dimension of feature space (Wold, 1987). When performing PCA, the information threshold is set to 0.95, which indicates that after dimension reduction, at least 95% of the original information should be preserved. By PCA, the dimension of feature space is reduced from 19 to 5.

### Modeling by Regression

### Regression Model: Linear Regression

Linear regression is performed on the five-dimensional data obtained from PCA in the above step. Note that in this case, there are only 26 sets of data available. Since linear regression is simple regression function with less regression coefficients, using linear regression can reduce the Possibility of overfitting.

Intercept: 3.1860075866538455 coefficient: [−0.03175369, −0.27855286, −0.03642713, 0.08569549, 0.45963195, −0.34116744]

### Regression Performance Evaluation

To evaluate the regression model, the standard approach is to split the data into the training set and testing set, usually with the ratio of 6:4. However, in this case, there are only have 26 sets of data, so simply splitting the data into training and testing sets may not be able to tell whether the model is overfitted. Therefore, a technique called cross-validation is used. Each time 15 data points are randomly picked as the training set, and the rest 11 as the testing set. The process is repeated for 10 times, and the error of the model is calculated by averaging the results of the 10 trials. As can be seen from the error scores of this model, the score varies a lot with different divisions of training and testing set, which indicates overfitting due to small data size.

−36.86732892, −2.31933821, −11.36632332, 65.89540659, −1.81236746, 0.42632973, −38.64697631, 0.84348793, −168.59368642, −3.72672957

### Implementation

The structural and non-structural parameters can be calculated from a python package called Vienna RNA. The Sciki-leam package is used for data analysis (Pedregosa, 2011).

### Software

### Function & Design

In the data analysis part of the last section, a regression function is calculated to predict the performance of a specific aptamer. The software introduced will make use of the structural information and scoring function mentioned above to help screen and select the aptamer with the best performance in prediction.

The input of the software is a .fasta file containing the viral RNA sequence. A screening window slides from the first position to the end of the whole sequence. In each position, the sequence inside the window is selected as a candidate for aptamer design and is evaluated based on its MFE structure and the scoring function discussed previously. Each time the window moves by one nucleotide. After reaching the endpoint of the sequence, the program output aptamer sequences with scores higher than the predefined threshold.

### Discussion

Traditionally, RNA aptamers are generated using the SELEX procedure, which takes a long time and has no guarantee of generating the optimum design. In this example, a rational way of designing RNA aptamer is proposed. Based on pairs of Spinach aptamers and targets, several structural and non-structural parameters that can potentially affect the performance of aptamer are identified. Structural features can help detect un-promising design, while non-structural ones can be used to derive a regression model. For non-structural features, feature selection based on mutual information is used to filter out irrelevant parameters, and principal component analysis to reduce data dimension. Multivariate linear regression is then performed in the reduced dimension to generate a scoring function that can be used to predict the performance (fold change) of aptamers. This model is used along with structural features in the designing software to help screen and select aptamer designs. As more data points are obtained, the prediction of the software will become more accurate. Besides Spinach-DHFBI and influenza virus, the same algorithm can potentially be applied to other fluorogens and other target RNA sequences.

### REFERENCES

1. Patel P, Graser E, Robst S, Hillert R, Meye A, Hillebrand T. Niedrig M. Rapid STRIPE H1N1 test for detection of the pandemic swine origin influenza A (H1N1) virus. J Clin Microbiol. 2011 April:49(4):1591-3.
2. Green A A, Silver P A, Collins J J, Yin P. Toehold switches: de-novo-designed regulators of gene expression. Cell. 2014 Nov. 6; 159(4):925-39.
3. Pardee K et. al. Rapid, Low-Cost Detection of Zika Virus Using Programmable Biomolecular Components. Cell. 2016 May 19; 165(5):1255-66.
4. Team Hong_Kong-CUHK [Internet]. iGEM. 2017 [cited 31 May 2018]. Available from: http://2017.igem.orgiTeam:Hong_Kong-CUHK
5. Bouhedda F, Autour A. Ryckelynck M. Light-up RNA aptamers and their cognate fluorogens: From their development to their applications. Int J Mol Sci. 2018; 19(1):44.
6. Kikuchi N, Kolpashchikov D M. Split Spinach Aptamer for Highly Selective Recognition of DNA and RNA at Ambient Temperatures. Chem Bio Chem. 2016 Jul. 15; 17(17):1589-92. Available from: http://dx.doi.org/10.1002/cbic.201600323
7. W. Q. Ong, Y. R. Citron, S. Sekine, and B. Huang, “Live Cell imaging of endogenous mRNA Using RNA-Based fluorescence ‘turn-on’ probe,” ACS Chem. Biol., vol. 12, no. 1, pp. 200-205, 2017.
8. K. Sato, M. Hamada, K. Asai, and T. Mituyama, “CentroidFold: a web server for RNA secondary structure prediction.” Nucleic Acids Research. vol. 37, Web Server issue W277-W280, 2009.
9. Hutchinson, Matthew H., et al. “Optical properties of polylactides.” Journal of Polymers and the Environment 14.2 (2006): 119-124.
10. AMC7135 datasheet https://wvw.electroschematics.conm/wp-contentluploads/2016/04/acm7135-datasheet.pdf
11. L. Ivo, and Hofacker, “Vienna RNA secondary structure server” Nucleic Acids Research, vol. 31, no. 13, pp. 3429-3431, 2003.
12. R. L. Strack, M. D. Disney, and S. R. Jaffrey, “A superfolding Spinach2 reveals the dynamic nature of trinucleotide repeat-containing RNA,” Nat. Methods, vol. 10, no. 12, pp. 1219-1224, 2013.
13. Open CV-Python Tutorials: https://opencv-python-tutroals.readthedocs.io/en/latest/pv_tutorials/pv_calib3d/pv_calibration/pv_calibration.html
14. 2017 iGem team: http://2017.igem.org/Team:Hong_Kong-CUHK/Software
15. Vienna RNA: https://www.tbi.univie.ac.at/RNA/
16. OpenCV camera calibration: https://opencv-python-tutroals.readthedocs.io/en/latest/pv_tutorials/pv_calib3d/pv_calibration/pv_calibration.html
17. 2018 iGem team:Hong_Kong-CUHK/Wetlab, http://2018.ieem.org/Team:Hong_Kong-CUHK/wetlab.
18. Duesberg, Peter H. “The RNA of influenza virus.” Proceedings of the National Academy of Sciences of the United States of America 59.3 (1968): 930.
19. Wold, S., Esbensen, K., & Geladi. P. (1987). Principal component analysis. Chemometrics and intelligent laboratory systems, 2(1-3), 37-52.
20. Pedregosa. F., Varoquaux, G., Gramfort, A., Michel, V., Thirion, B., Grisel, O., . . . & Vanderplas. J. (2011). Scikit-leam: Machine learning in Python. the Journal of machine Learning research, 12, 2825-2830.
21. Nie, Shuping, et al. “Evaluation of Alere i Influenza A&B for rapid detection of influenza viruses A and B.” Journal of clinical microbiology 52.9 (2014): 3339-3344.

What is claimed is:

1. A method for designing a probe for detecting a target sequence of nuclei acid in presence of a fluorogen, comprising the steps of:
a. selecting a target sequence;
b. selecting an aptamer sequence for forming a secondary structure comprising a fluorogen docking site that is destabilized;
c. generating one or more detecting sequences substantially complementary to a region on said target sequence and adding said one or more detecting sequences to an end of said aptamer sequence to form a probe sequence;
d. determining binding probability between complementary pairs of nucleotides in said probe sequence responsible for stabilizing of said fluorogen docking site;
e. obtaining value of one or more non-structural features related to said probe sequence and said target sequence;
f. obtaining a first value indicative of probability of forming a heterodimer of said probe sequence and said target sequence from the results of (e);
g. obtaining a second value indicative of probability of autofluorescence of said probe sequence from the results of (d); and
h. determining if said probe sequence is a suitable probe candidate based on said first and second values.

2. The method of claim 1, wherein said non-structural features comprises:
a. minimal free energy of said heterodimer;
b. minimal free energy of a homodimer of said probe sequence;
c. minimal free energy of a homodimer of said target sequence;
d. value of delta G for binding of said heterodimer; and
e. frequency of minimal free energy structure of said heterodimer.

3. The method of claim 1, wherein said first value is obtained from the following equation:
first value=A(minimal free energy of homodimer of said probe sequence)+B (minimal free energy of homodimer of said target sequence)+C (minimal free energy of heterodimer of said probe sequence and said target sequence)+D (value of delta G for binding of heterodimer of said probe sequence and said target sequence)+E (frequency of minimal free energy structure of said heterodimer)
where, A, B, C, D and E are coefficients obtained by multiple linear regression based on the following equation:
on/off ratio=contant+first value+error.

4. The method of claim 1, wherein said second value is sum of binding probabilities between complementary pairs of nucleotides in said probe sequence responsible for stabilizing of said fluorogen docking site, wherein binding probability of each of said complementary pairs of nucleotides has a specific coefficient obtained by multiple linear regression based on the following equation:
mean fluorescent count=constant+second value+error.

5. The method of claim 1, wherein said aptamer sequence comprises SEQ ID NO: 143.

6. The method of claim 1, wherein said aptamer sequence comprises SEQ ID NO: 1 and SEQ ID NO: 5 to form a P1 arm linked to said fluorogen docking site.

7. The method of claim 6, wherein said complementary pairs of nucleotides of step (d) comprises the nucleotides 1 to 4 of SEQ ID NO: 1 being complementary to nucleotides 7 to 4 of SEQ ID NO: 5 respectively.

8. The method of claim 1, wherein said one or more detecting sequences comprises two detecting sequences, each linked to an end of said aptamer sequence.

9. The method of claim 1, wherein said one or more non-structural features of step (e) is identified by:
a. determining normalized mutual information scores for a plurality of non-structural features of said probe sequence to identify a shortlist of non-structural features; and
b. conducting principal component analysis on said shortlist of non-structural features to identify said one or more non-structural features of step (e).

10. The method of claim 1, wherein said target sequence is a region in the genome of a pathogen.

11. The method of claim 9, wherein said pathogen is an RNA virus.

12. The method of claim 11, wherein said RNA virus is selected from the group consisting of influenza virus, SARS-CoV, Zika virus and hepatitis C virus.

13. The method of claim 9, further comprising the step of experimentally validating said probe sequence of step (h) and fine tuning said first and second values of steps (f) and (g).

14. A probe designed based on the method of claim 1.

15. The probe of claim 14, wherein said probe sequence comprises:
a. an RNA selected from SEQ ID NOs: 143-170; or
b. an RNA obtained by DNA transcription of SEQ ID NOs: 6-32.

16. The probe of claim 14, wherein said one or more detecting sequences comprise a sequence selected from the group of SEQ ID NOs: 88-141.

17. The probe of claim 14, wherein said fluorogen is 3,5-difluoro-4-hydroxybenzylidene imidazolinone (DFHBI).

18. A probe for detecting a target sequence of nuclei acid in presence of a fluorogen, comprising:
a. an aptamer sequence comprising SEQ ID NO: 143; and
b. one or more detecting sequences comprising SEQ ID NOs: 88-141.

19. The probe of claim 18, wherein said probe comprises:
a. an RNA obtained by DNA transcription of SEQ ID NOs: 6-32; or
b. an RNA selected from SEQ ID NOs: 144-170.

20. The probe of claim 18, wherein said one or more detecting sequences are two detecting sequences, each linked to an end of said aptamer sequence and complimentary to a continuous region on said target sequence.


SEQUENCE LISTING (171 sequences)

SEQ ID NO: 1 - RNA, 6 nt, Artificial Sequence
  RNA aptamer probe P1 arm sequence
ggcgaa

SEQ ID NO: 2 - RNA, 7 nt, Artificial Sequence
  RNA aptamer probe docking site sequence
ggacggg

SEQ ID NO: 3 - RNA, 19 nt, Artificial Sequence
  RNA aptamer probe P2 arm sequence
uccagcguucgcgcuguug

SEQ ID NO: 4 - RNA, 11 nt, Artificial Sequence
  RNA aptamer probe docking site sequence
aguagagugug

SEQ ID NO: 5 - RNA, 6 nt, Artificial Sequence
  RNA aptamer probe P1 arm sequence
agcgcc

SEQ ID NO: 6 - DNA, 71 bp, Artificial Sequence
  Original DNA full sequence of RNA aptamer probe N1-86
gtttggattgaggcgaaggacgggtccagcgttcgcgctgttgagtagagtgtgagcgccgtgactagccc

SEQ ID NO: 7 - DNA, 71 bp, Artificial Sequence
  Original DNA full sequence of RNA aptamer probe N1-167
acatatgtgtgggcgaaggacgggtccagcgttcgcgctgttgagtagagtgtgagcgccattcacccagg

SEQ ID NO: 8 - DNA, 71 bp, Artificial Sequence
  Original DNA full sequence of RNA aptamer probe N1-411
ggtcccatttgggcgaaggacgggtccagcgttcgcgctgttgagtagagtgtgagcgccaatgtttgtca

SEQ ID NO: 9 - DNA, 71 bp, Artificial Sequence
  Original DNA full sequence of RNA aptamer probe N1-862
aaccatgccagggcgaaggacgggtccagcgttcgcgctgttgagtagagtgtgagcgccttgtccctgca

SEQ ID NO: 10 - DNA, 71 bp, Artificial Sequence
  Original DNA full sequence of RNA aptamer probe N9-369
agcatagaaccggcgaaggacgggtccagcgttcgcgctgttgagtagagtgtgagcgcctgcattcatct

SEQ ID NO: 11 - DNA, 71 bp, Artificial Sequence
  Original DNA full sequence of RNA aptamer probe N9-531
ccatcgtggcaggcgaaggacgggtccagcgttcgcgctgttgagtagagtgtgagcgccactagtacttg

SEQ ID NO: 12 - DNA, 71 bp, Artificial Sequence
  Original DNA full sequence of RNA aptamer probe N9-545
acatcctggatggcgaaggacgggtccagcgttcgcgctgttgagtagagtgtgagcgccttaccatcgtg

SEQ ID NO: 13 - DNA, 71 bp, Artificial Sequence
  Original DNA full sequence of RNA aptamer probe N9-868
tgagccctgccggcgaaggacgggtccagcgttcgcgctgttgagtagagtgtgagcgccaattgtccctg

SEQ ID NO: 14 - DNA, 71 bp, Artificial Sequence
  Original DNA full sequence of RNA aptamer probe N2-165
gttggttcacaggcgaaggacgggtccagcgttcgcgctgttgagtagagtgtgagcgcccagcatcactt

SEQ ID NO: 15 - DNA, 71 bp, Artificial Sequence
  Original DNA full sequence of RNA aptamer probe N2-530
atgctatgcacggcgaaggacgggtccagcgttcgcgctgttgagtagagtgtgagcgccacttgcttggt

SEQ ID NO: 16 - DNA, 71 bp, Artificial Sequence
  Original DNA full sequence of RNA aptamer probe N2-694
acaaacgcattggcgaaggacgggtccagcgttcgcgctgttgagtagagtgtgagcgccctgactcctgg

SEQ ID NO: 17 - DNA, 71 bp, Artificial Sequence
  Original DNA full sequence of RNA aptamer probe N2-894
ttggagcctttggcgaaggacgggtccagcgttcgcgctgttgagtagagtgtgagcgccccagttgtctc

SEQ ID NO: 18 - DNA, 71 bp, Artificial Sequence
  Original DNA full sequence of RNA aptamer probe H1-483
cgtcagccataggcgaaggacgggtccagcgttcgcgctgttgagtagagtgtgagcgccgcaaatttttg

SEQ ID NO: 19 - DNA, 71 bp, Artificial Sequence
  Original DNA full sequence of RNA aptamer probe H1-660
ggtgaatttccggcgaaggacgggtccagcgttcgcgctgttgagtagagtgtgagcgcctgctataatgt

SEQ ID NO: 20 - DNA, 71 bp, Artificial Sequence
  Original DNA full sequence of RNA aptamer probe H1-825
gatgattcctgggcgaaggacgggtccagcgttcgcgctgttgagtagagtgtgagcgccatccaaagcct

SEQ ID NO: 21 - DNA, 71 bp, Artificial Sequence
  Original DNA full sequence of RNA aptamer probe H3-416
aaactccagtgggcgaaggacgggtccagcgttcgcgctgttgagtagagtgtgagcgcctgccggatgag

SEQ ID NO: 22 - DNA, 71 bp, Artificial Sequence
  Original DNA full sequence of RNA aptamer probe H3-757
caatagatgctggcgaaggacgggtccagcgttcgcgctgttgagtagagtgtgagcgcctattctgctgg

SEQ ID NO: 23 - DNA, 71 bp, Artificial Sequence
  Original DNA full sequence of RNA aptamer probe H3-758
ccaatagatgcggcgaaggacgggtccagcgttcgcgctgttgagtagagtgtgagcgccttattctgctg

SEQ ID NO: 24 - DNA, 71 bp, Artificial Sequence
  Original DNA full sequence of RNA aptamer probe H3-1305
tagtgtcctcaggcgaaggacgggtccagcgttcgcgctgttgagtagagtgtgagcgccacatatttctc

SEQ ID NO: 25 - DNA, 71 bp, Artificial Sequence
  Original DNA full sequence of RNA aptamer probe H7-474
tgacaggagccggcgaaggacgggtccagcgttcgcgctgttgagtagagtgtgagcgccatttcatttct

SEQ ID NO: 26 - DNA, 71 bp, Artificial Sequence
  Original DNA full sequence of RNA aptamer probe H7-637
attagaactccggcgaaggacgggtccagcgttcgcgctgttgagtagagtgtgagcgcccaactgtcacc

SEQ ID NO: 27 - DNA, 71 bp, Artificial Sequence
  Original DNA full sequence of RNA aptamer probe H7-714
caatgaaagtcggcgaaggacgggtccagcgttcgcgctgttgagtagagtgtgagcgccaattcttccgg

SEQ ID NO: 28 - DNA, 71 bp, Artificial Sequence
  Original DNA full sequence of RNA aptamer probe H7-831
acctgtacaccggcgaaggacgggtccagcgttcgcgctgttgagtagagtgtgagcgccactctggattc

SEQ ID NO: 29 - DNA, 71 bp, Artificial Sequence
  Original DNA full sequence of RNA aptamer probe PB2-2209
ttaccaacaccggcgaaggacgggtccagcgttcgcgctgttgagtagagtgtgagcgccacgtctccttg

SEQ ID NO: 30 - DNA, 71 bp, Artificial Sequence
  Original DNA full sequence of RNA aptamer probe PB2-2247
agagttccggcggcgaaggacgggtccagcgttcgcgctgttgagtagagtgtgagcgcctagagttccgt

SEQ ID NO: 31 - DNA, 71 bp, Artificial Sequence
  Original DNA full sequence of RNA aptamer probe PB2-2265
gctgtcagtaaggcgaaggacgggtccagcgttcgcgctgttgagtagagtgtgagcgccgtatgctagag

SEQ ID NO: 32 - DNA, 71 bp, Artificial Sequence
  Original DNA full sequence of RNA aptamer probe PB2-2300
cttttaattctggcgaaggacgggtccagcgttcgcgctgttgagtagagtgtgagcgcctttggtcgctg

SEQ ID NO: 33 - DNA, 51 bp, Artificial Sequence
  Original DNA full sequence of RNA aptamer probe miniSpinach (P1-a5-b3)
gggagaaggacgggtccagcgttcgcgctgttgagtagagtgtgagctccc

SEQ ID NO: 34 - RNA, 11 nt, Influenza A virus
caaaccuaacu

SEQ ID NO: 35 - RNA, 11 nt, Influenza A virus
cacugaucggg

SEQ ID NO: 36 - RNA, 11 nt, Influenza A virus
uguauacacac

SEQ ID NO: 37 - RNA, 11 nt, Influenza A virus
uaagugggucc

SEQ ID NO: 38 - RNA, 11 nt, Influenza A virus
ccaggguaaac

SEQ ID NO: 39 - RNA, 11 nt, Influenza A virus
uuacaaacagu

SEQ ID NO: 40 - RNA, 11 nt, Influenza A virus
uugguacgguc

SEQ ID NO: 41 - RNA, 11 nt, Influenza A virus
aacagggacgu

SEQ ID NO: 42 - RNA, 11 nt, Influenza A virus
ucguaucuugg

SEQ ID NO: 43 - RNA, 11 nt, Influenza A virus
acguaaguaga

SEQ ID NO: 44 - RNA, 11 nt, Influenza A virus
gguagcaccgu

SEQ ID NO: 45 - RNA, 11 nt, Influenza A virus
ugaucaugaac

SEQ ID NO: 46 - RNA, 11 nt, Influenza A virus
uguaggaccua

SEQ ID NO: 47 - RNA, 11 nt, Influenza A virus
aaugguagcac

SEQ ID NO: 48 - RNA, 11 nt, Influenza A virus
acucgggacgg

SEQ ID NO: 49 - RNA, 11 nt, Influenza A virus
uuaacagggac

SEQ ID NO: 50 - RNA, 11 nt, Influenza A virus
caaccaagugu

SEQ ID NO: 51 - RNA, 11 nt, Influenza A virus
gucguagugaa

SEQ ID NO: 52 - RNA, 11 nt, Influenza A virus
uacgauacgug

SEQ ID NO: 53 - RNA, 11 nt, Influenza A virus
ugaacgaacca

SEQ ID NO: 54 - RNA, 11 nt, Influenza A virus
uguuugcguaa

SEQ ID NO: 55 - RNA, 11 nt, Influenza A virus
gacugaggacc

SEQ ID NO: 56 - RNA, 11 nt, Influenza A virus
aaccucggaaa

SEQ ID NO: 57 - RNA, 11 nt, Influenza A virus
ggucaacagag

SEQ ID NO: 58 - RNA, 11 nt, Influenza A virus
gcagucgguau

SEQ ID NO: 59 - RNA, 11 nt, Influenza A virus
cguuuaaaaac

SEQ ID NO: 60 - RNA, 11 nt, Influenza A virus
ccacuuaaagg

SEQ ID NO: 61 - RNA, 11 nt, Influenza A virus
acgauauuaca

SEQ ID NO: 62 - RNA, 11 nt, Influenza A virus
cuacuaaggac

SEQ ID NO: 63 - RNA, 11 nt, Influenza A virus
uagguuucgga

SEQ ID NO: 64 - RNA, 11 nt, Influenza A virus
uuugaggucac

SEQ ID NO: 65 - RNA, 11 nt, Influenza A virus
acggccuacuc

SEQ ID NO: 66 - RNA, 11 nt, Influenza A virus
guuaucuacga

SEQ ID NO: 67 - RNA, 11 nt, Influenza A virus
auaagacgacc

SEQ ID NO: 68 - RNA, 11 nt, Influenza A virus
gguuaucuacg

SEQ ID NO: 69 - RNA, 11 nt, Influenza A virus
aauaagacgac

SEQ ID NO: 70 - RNA, 11 nt, Influenza A virus
aucacaggagu

SEQ ID NO: 71 - RNA, 11 nt, Influenza A virus
uguauaaagag

SEQ ID NO: 72 - RNA, 11 nt, Influenza A virus
acuguccucgg

SEQ ID NO: 73 - RNA, 11 nt, Influenza A virus
uaaaguaaaga

SEQ ID NO: 74 - RNA, 11 nt, Influenza A virus
uaaucuugagg

SEQ ID NO: 75 - RNA, 11 nt, Influenza A virus
guugacagugg

SEQ ID NO: 76 - RNA, 11 nt, Influenza A virus
guuacuuucag

SEQ ID NO: 77 - RNA, 11 nt, Influenza A virus
uuaagaaggcc

SEQ ID NO: 78 - RNA, 11 nt, Influenza A virus
uggacaugugg

SEQ ID NO: 79 - RNA, 11 nt, Influenza A virus
ugagaccuaag

SEQ ID NO: 80 - RNA, 11 nt, Influenza A virus
aaugguugugg

SEQ ID NO: 81 - RNA, 11 nt, Influenza A virus
ugcagaggaac

SEQ ID NO: 82 - RNA, 11 nt, Influenza A virus
ucucaaggccg

SEQ ID NO: 83 - RNA, 11 nt, Influenza A virus
aucucaaggca

SEQ ID NO: 84 - RNA, 11 nt, Influenza A virus
cgacagucauu

SEQ ID NO: 85 - RNA, 11 nt, Influenza A virus
cauacgaucuc

SEQ ID NO: 86 - RNA, 11 nt, Influenza A virus
gaaaauuaaga

SEQ ID NO: 87 - RNA, 11 nt, Influenza A virus
aaaccagcgac

SEQ ID NO: 88 - RNA, 11 nt, Artificial Sequence
  Targeting sequence in P1 arm of N1-86 probe
guuuggauuga

SEQ ID NO: 89 - RNA, 11 nt, Artificial Sequence
  Targeting sequence in P1 arm of N1-86 probe
gugacuagccc

SEQ ID NO: 90 - RNA, 11 nt, Artificial Sequence
  Targeting sequence in P1 arm of N1-167 probe
acauaugugug

SEQ ID NO: 91 - RNA, 11 nt, Artificial Sequence
  Targeting sequence in P1 arm of N1-167 probe
auucacccagg

SEQ ID NO: 92 - RNA, 11 nt, Artificial Sequence
  Targeting sequence in P1 arm of N1-411 probe
ggucccauuug

SEQ ID NO: 93 - RNA, 11 nt, Artificial Sequence
  Targeting sequence in P1 arm of N1-411 probe
aauguuuguca

SEQ ID NO: 94 - RNA, 11 nt, Artificial Sequence
  Targeting sequence in P1 arm of N1-862 probe
aaccaugccag

SEQ ID NO: 95 - RNA, 11 nt, Artificial Sequence
  Targeting sequence in P1 arm of N1-862 probe
uugucccugca

SEQ ID NO: 96 - RNA, 11 nt, Artificial Sequence
  Targeting sequence in P1 arm of N9-369 probe
agcauagaacc

SEQ ID NO: 97 - RNA, 11 nt, Artificial Sequence
  Targeting sequence in P1 arm of N9-369 probe
ugcauucaucu

SEQ ID NO: 98 - RNA, 11 nt, Artificial Sequence
  Targeting sequence in P1 arm of N9-531 probe
ccaucguggca

SEQ ID NO: 99 - RNA, 11 nt, Artificial Sequence
  Targeting sequence in P1 arm of N9-531 probe
acuaguacuug

SEQ ID NO: 100 - RNA, 11 nt, Artificial Sequence
  Targeting sequence in P1 arm of N9-545 probe
acauccuggau

SEQ ID NO: 101 - RNA, 11 nt, Artificial Sequence
  Targeting sequence in P1 arm of N9-545 probe
uuaccaucgug

SEQ ID NO: 102 - RNA, 11 nt, Artificial Sequence
  Targeting sequence in P1 arm of N9-868 probe
ugagcccugcc

SEQ ID NO: 103 - RNA, 11 nt, Artificial Sequence
  Targeting sequence in P1 arm of N9-868 probe
aauugucccug

SEQ ID NO: 104 - RNA, 11 nt, Artificial Sequence
  Targeting sequence in P1 arm of N2-165 probe
guugguucaca

SEQ ID NO: 105 - RNA, 11 nt, Artificial Sequence
  Targeting sequence in P1 arm of N2-165 probe
cagcaucacuu

SEQ ID NO: 106 - RNA, 11 nt, Artificial Sequence
  Targeting sequence in P1 arm of N2-530 probe
augcuaugcac

SEQ ID NO: 107 - RNA, 11 nt, Artificial Sequence
  Targeting sequence in P1 arm of N2-530 probe
acuugcuuggu

SEQ ID NO: 108 - RNA, 11 nt, Artificial Sequence
  Targeting sequence in P1 arm of N2-694 probe
acaaacgcauu

SEQ ID NO: 109 - RNA, 11 nt, Artificial Sequence
  Targeting sequence in P1 arm of N2-694 probe
cugacuccugg

SEQ ID NO: 110 - RNA, 11 nt, Artificial Sequence
  Targeting sequence in P1 arm of N2-894 probe
uuggagccuuu

SEQ ID NO: 111 - RNA, 11 nt, Artificial Sequence
  Targeting sequence in P1 arm of N2-894 probe
ccaguugucuc

SEQ ID NO: 112 - RNA, 11 nt, Artificial Sequence
  Targeting sequence in P1 arm of H1-483 probe
cgucagccaua

SEQ ID NO: 113 - RNA, 11 nt, Artificial Sequence
  Targeting sequence in P1 arm of H1-483 probe
gcaaauuuuug

SEQ ID NO: 114 - RNA, 11 nt, Artificial Sequence
  Targeting sequence in P1 arm of H1-660 probe
ggugaauuucc

SEQ ID NO: 115 - RNA, 11 nt, Artificial Sequence
  Targeting sequence in P1 arm of H1-660 probe
ugcuauaaugu

SEQ ID NO: 116 - RNA, 11 nt, Artificial Sequence
  Targeting sequence in P1 arm of H1-825 probe
gaugauuccug

SEQ ID NO: 117 - RNA, 11 nt, Artificial Sequence
  Targeting sequence in P1 arm of H1-825 probe
auccaaagccu

SEQ ID NO: 118 - RNA, 11 nt, Artificial Sequence
  Targeting sequence in P1 arm of H3-416 probe
aaacuccagug

SEQ ID NO: 119 - RNA, 11 nt, Artificial Sequence
  Targeting sequence in P1 arm of H3-416 probe
ugccggaugag

SEQ ID NO: 120 - RNA, 11 nt, Artificial Sequence
  Targeting sequence in P1 arm of H3-757 probe
caauagaugcu

SEQ ID NO: 121 - RNA, 11 nt, Artificial Sequence
  Targeting sequence in P1 arm of H3-757 probe
uauucugcugg

SEQ ID NO: 122 - RNA, 11 nt, Artificial Sequence
  Targeting sequence in P1 arm of H3-758 probe
ccaauagaugc

SEQ ID NO: 123 - RNA, 11 nt, Artificial Sequence
  Targeting sequence in P1 arm of H3-758 probe
uuauucugcug

SEQ ID NO: 124 - RNA, 11 nt, Artificial Sequence
  Targeting sequence in P1 arm of H3-1305 probe
uaguguccuca

SEQ ID NO: 125 - RNA, 11 nt, Artificial Sequence
  Targeting sequence in P1 arm of H3-1305 probe
acauauuucuc

SEQ ID NO: 126 - RNA, 11 nt, Artificial Sequence
  Targeting sequence in P1 arm of H7-474 probe
ugacaggagcc

SEQ ID NO: 127 - RNA, 11 nt, Artificial Sequence
  Targeting sequence in P1 arm of H7-474 probe
auuucauuucu

SEQ ID NO: 128 - RNA, 11 nt, Artificial Sequence
  Targeting sequence in P1 arm of H7-637 probe
auuagaacucc

SEQ ID NO: 129 - RNA, 11 nt, Artificial Sequence
  Targeting sequence in P1 arm of H7-637 probe
caacugucacc

SEQ ID NO: 130 - RNA, 11 nt, Artificial Sequence
  Targeting sequence in P1 arm of H7-714 probe
caaugaaaguc

SEQ ID NO: 131 - RNA, 11 nt, Artificial Sequence
  Targeting sequence in P1 arm of H7-714 probe
aauucuuccgg

SEQ ID NO: 132 - RNA, 11 nt, Artificial Sequence
  Targeting sequence in P1 arm of H7-831 probe
accuguacacc

SEQ ID NO: 133 - RNA, 11 nt, Artificial Sequence
  Targeting sequence in P1 arm of H7-831 probe
acucuggauuc

SEQ ID NO: 134 - RNA, 11 nt, Artificial Sequence
  Targeting sequence in P1 arm of PB2-2209 probe
uuaccaacacc

SEQ ID NO: 135 - RNA, 11 nt, Artificial Sequence
  Targeting sequence in P1 arm of PB2-2209 probe
acgucuccuug

SEQ ID NO: 136 - RNA, 11 nt, Artificial Sequence
  Targeting sequence in P1 arm of PB2-2247 probe
agaguuccggc

SEQ ID NO: 137 - RNA, 11 nt, Artificial Sequence
  Targeting sequence in P1 arm of PB2-2247 probe
uagaguuccgu

SEQ ID NO: 138 - RNA, 11 nt, Artificial Sequence
  Targeting sequence in P1 arm of PB2-2265 probe
gcugucaguaa

SEQ ID NO: 139 - RNA, 11 nt, Artificial Sequence
  Targeting sequence in P1 arm of PB2-2265 probe
guaugcuagag

SEQ ID NO: 140 - RNA, 11 nt, Artificial Sequence
  Targeting sequence in P1 arm of PB2-2300 probe
cuuuuaauucu

SEQ ID NO: 141 - RNA, 11 nt, Artificial Sequence
  Targeting sequence in P1 arm of PB2-2230 probe
uuuggucgcug

SEQ ID NO: 142 - RNA, 71 nt, Artificial Sequence
  N9-545 aptamer sequence
acauccuggauggcgaaggacggguccagcguucgcgcuguugaguagagugugagcgccuuaccaucgug

SEQ ID NO: 143 - RNA, 49 nt, Artificial Sequence
  Aptamer sequence
  misc_feature (14)..(14): u or absent
  misc_feature (15)..(15): c or absent
  misc_feature (16)..(16): c or absent
  misc_feature (17)..(17): a or absent
  misc_feature (18)..(18): g or absent
  misc_feature (19)..(19): c or absent
  misc_feature (20)..(20): g or absent
  misc_feature (21)..(21): u or absent
  misc_feature (22)..(22): u or absent
  misc_feature (23)..(23): c or absent
  misc_feature (24)..(24): g or absent
  misc_feature (25)..(25): c or absent
  misc_feature (26)..(26): g or absent
  misc_feature (27)..(27): c or absent
  misc_feature (28)..(28): u or absent
  misc_feature (29)..(29): g or absent
  misc_feature (30)..(30): u or absent
  misc_feature (31)..(31): u or absent
  misc_feature (32)..(32): g or absent
ggcgaaggacgggnnnnnnnnnnnnnnnnnnnaguagagugugagcgcc

SEQ ID NO: 144 - RNA, 71 nt, Artificial Sequence
  RNA aptamer probe N1-86 full sequence
guuuggauugaggcgaaggacggguccagcguucgcgcuguugaguagagugugagcgccgugacuagccc

SEQ ID NO: 145 - RNA, 71 nt, Artificial Sequence
  RNA aptamer probe N1-167 full sequence
acauaugugugggcgaaggacggguccagcguucgcgcuguugaguagagugugagcgccauucacccagg

SEQ ID NO: 146 - RNA, 71 nt, Artificial Sequence
  RNA aptamer probe N1-411 full sequence
ggucccauuugggcgaaggacggguccagcguucgcgcuguugaguagagugugagcgccaauguuuguca

SEQ ID NO: 147 - RNA, 71 nt, Artificial Sequence
  RNA aptamer probe N1-862 full sequence
aaccaugccagggcgaaggacggguccagcguucgcgcuguugaguagagugugagcgccuugucccugca

SEQ ID NO: 148 - RNA, 71 nt, Artificial Sequence
  RNA aptamer probe N9-369 full sequence
agcauagaaccggcgaaggacggguccagcguucgcgcuguugaguagagugugagcgccugcauucaucu

SEQ ID NO: 149 - RNA, 71 nt, Artificial Sequence
  RNA aptamer probe N9-531 full sequence
ccaucguggcaggcgaaggacggguccagcguucgcgcuguugaguagagugugagcgccacuaguacuug

SEQ ID NO: 150 - RNA, 71 nt, Artificial Sequence; identical to SEQ ID NO: 142
  RNA aptamer probe N9-545 full sequence

SEQ ID NO: 151 - RNA, 71 nt, Artificial Sequence
  RNA aptamer probe N9-868 full sequence
ugagcccugccggcgaaggacggguccagcguucgcgcuguugaguagagugugagcgccaauugucccug

SEQ ID NO: 152 - RNA, 71 nt, Artificial Sequence
  RNA aptamer probe N2-165 full sequence
guugguucacaggcgaaggacggguccagcguucgcgcuguugaguagagugugagcgcccagcaucacuu

SEQ ID NO: 153 - RNA, 71 nt, Artificial Sequence
  RNA aptamer probe N2-530 full sequence
augcuaugcacggcgaaggacggguccagcguucgcgcuguugaguagagugugagcgccacuugcuuggu

SEQ ID NO: 154 - RNA, 71 nt, Artificial Sequence
  RNA aptamer probe N2-694 full sequence
acaaacgcauuggcgaaggacggguccagcguucgcgcuguugaguagagugugagcgcccugacuccugg

SEQ ID NO: 155 - RNA, 71 nt, Artificial Sequence
  RNA aptamer probe N2-894 full sequence
uuggagccuuuggcgaaggacggguccagcguucgcgcuguugaguagagugugagcgccccaguugucuc

SEQ ID NO: 156 - RNA, 71 nt, Artificial Sequence
  RNA aptamer probe H1-483 full sequence
cgucagccauaggcgaaggacggguccagcguucgcgcuguugaguagagugugagcgccgcaaauuuuug

SEQ ID NO: 157 - RNA, 71 nt, Artificial Sequence
  RNA aptamer probe H1-660 full sequence
ggugaauuuccggcgaaggacggguccagcguucgcgcuguugaguagagugugagcgccugcuauaaugu

SEQ ID NO: 158 - RNA, 71 nt, Artificial Sequence
  RNA aptamer probe H1-825 full sequence
gaugauuccugggcgaaggacggguccagcguucgcgcuguugaguagagugugagcgccauccaaagccu

SEQ ID NO: 159 - RNA, 71 nt, Artificial Sequence
  RNA aptamer probe H3-416 full sequence
aaacuccagugggcgaaggacggguccagcguucgcgcuguugaguagagugugagcgccugccggaugag

SEQ ID NO: 160 - RNA, 71 nt, Artificial Sequence
  RNA aptamer probe H3-757 full sequence
caauagaugcuggcgaaggacggguccagcguucgcgcuguugaguagagugugagcgccuauucugcugg

SEQ ID NO: 161 - RNA, 71 nt, Artificial Sequence
  RNA aptamer probe H3-758 full sequence
ccaauagaugcggcgaaggacggguccagcguucgcgcuguugaguagagugugagcgccuuauucugcug

SEQ ID NO: 162 - RNA, 71 nt, Artificial Sequence
  RNA aptamer probe H3-1305 full sequence
uaguguccucaggcgaaggacggguccagcguucgcgcuguugaguagagugugagcgccacauauuucuc

SEQ ID NO: 163 - RNA, 71 nt, Artificial Sequence
  RNA aptamer probe H7-474 full sequence
ugacaggagccggcgaaggacggguccagcguucgcgcuguugaguagagugugagcgccauuucauuucu

SEQ ID NO: 164 - RNA, 71 nt, Artificial Sequence
  RNA aptamer probe H7-637 full sequence
auuagaacuccggcgaaggacggguccagcguucgcgcuguugaguagagugugagcgcccaacugucacc

SEQ ID NO: 165 - RNA, 71 nt, Artificial Sequence
  RNA aptamer probe H7-714 full sequence
caaugaaagucggcgaaggacggguccagcguucgcgcuguugaguagagugugagcgccaauucuuccgg

SEQ ID NO: 166 - RNA, 71 nt, Artificial Sequence
  RNA aptamer probe H7-831 full sequence
accuguacaccggcgaaggacggguccagcguucgcgcuguugaguagagugugagcgccacucuggauuc

SEQ ID NO: 167 - RNA, 71 nt, Artificial Sequence
  RNA aptamer probe PB2-2209 full sequence
uuaccaacaccggcgaaggacggguccagcguucgcgcuguugaguagagugugagcgccacgucuccuug

SEQ ID NO: 168 - RNA, 71 nt, Artificial Sequence
  RNA aptamer probe PB2-2247 full sequence
agaguuccggcggcgaaggacggguccagcguucgcgcuguugaguagagugugagcgccuagaguuccgu

SEQ ID NO: 169 - RNA, 71 nt, Artificial Sequence
  RNA aptamer probe PB2-2265 full sequence
gcugucaguaaggcgaaggacggguccagcguucgcgcuguugaguagagugugagcgccguaugcuagag

SEQ ID NO: 170 - RNA, 71 nt, Artificial Sequence
  RNA aptamer probe PB2-2300 full sequence
cuuuuaauucuggcgaaggacggguccagcguucgcgcuguugaguagagugugagcgccuuuggucgcug

SEQ ID NO: 171 - RNA, 51 nt, Artificial Sequence
  RNA aptamer probe miniSpinach (P1-a5-b3) full sequence
gggagaaggacggguccagcguucgcgcuguugaguagagugugagcuccc